SEQUENCE LISTING (23 sequences)

SEQ ID NO: 1 - protein, 21 aa, Homo sapiens
  source 1..21: note = Ciltacabtagene autoleucel CAR CD8alpha signal peptide, CD8alpha SP amino acid sequence
MALPVTALLLPLALLLHAARP

SEQ ID NO: 2 - protein, 119 aa, Lama glama
  source 1..119: note = Ciltacabtagene autoleucel CAR BCMA binding domain, VHH1 amino acid sequence
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 3 - protein, 5 aa, synthetic construct
  source 1..5: note = Ciltacabtagene autoleucel CAR BCMA binding domain, G4S linker amino acid sequence
GGGGS

SEQ ID NO: 4 - protein, 118 aa, Lama glama
  source 1..118: note = Ciltacabtagene autoleucel CAR BCMA binding domain, VHH2 amino acid sequence
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 5 - protein, 45 aa, Homo sapiens
  source 1..45: note = Ciltacabtagene autoleucel CAR CD8alpha hinge amino acid sequence
TTTPAPRPPTPAPTIASQPLSLRPEACRPAAGGAVHTRGLDFACD

SEQ ID NO: 6 - protein, 24 aa, Homo sapiens
  source 1..24: note = Ciltacabtagene autoleucel CAR CD8alpha transmembrane amino acid sequence
IYIWAPLAGTCGVLLLSLVITLYC

SEQ ID NO: 7 - protein, 42 aa, Homo sapiens
  source 1..42: note = Ciltacabtagene autoleucel CAR CD137 Cytoplasmic amino acid sequence
KRGRKKLLYIFKQPFMRPVQTTQEEDGCSCRFPEEEEGGCEL

SEQ ID NO: 8 - protein, 112 aa, Homo sapiens
  source 1..112: note = Ciltacabtagene autoleucel CAR CD3zeta Cytoplasmic amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, Homo sapiens
  source 1..63: mol_type = other DNA; note = Ciltacabtagene autoleucel CAR CD8alpha signal peptide CD8alpha SP nucleic acid sequence
atggctctgcccgtcaccgctctgctgctgcctctggctctgctgctgcacgctgctcgccct

SEQ ID NO: 10 - DNA, 357 bp, Lama glama
  source 1..357: mol_type = other DNA; note = Ciltacabtagene autoleucel CAR BCMA binding domain, VHH1 nucleic acid sequence
caggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagc

SEQ ID NO: 11 - DNA, 15 bp, synthetic construct
  source 1..15: mol_type = other DNA; note = Ciltacabtagene autoleucel CAR BCMA binding domain, G4S linker nucleic acid sequence
ggaggaggaggatct

SEQ ID NO: 12 - DNA, 354 bp, Lama glama
  source 1..354: mol_type = other DNA; note = Ciltacabtagene autoleucel CAR BCMA binding domain, VHH2 nucleic acid sequence
gaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagc

SEQ ID NO: 13 - DNA, 135 bp, Homo sapiens
  source 1..135: mol_type = other DNA; note = Ciltacabtagene autoleucel CAR CD8alpha hinge nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 72 bp, Homo sapiens
  source 1..72: mol_type = other DNA; note = Ciltacabtagene autoleucel CAR CD8alpha transmembrane nucleic acid sequence
atctacatctgggcgcccttggccgggacttgtggggtccttctcctgtcactggttatcaccctttactgc

SEQ ID NO: 15 - DNA, 126 bp, Homo sapiens
  source 1..126: mol_type = other DNA; note = Ciltacabtagene autoleucel CAR CD137 Cytoplasmic nucleic acid sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - DNA, 339 bp, Homo sapiens
  source 1..339: mol_type = other DNA; note = Ciltacabtagene autoleucel CAR CD3zeta Cytoplasmic nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - protein, 488 aa, synthetic construct
  source 1..488: note = Ciltacabtagene autoleucel CAR amino acid sequence
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 5 aa, Lama glama
  source 1..5: note = Ciltacabtagene autoleucel CAR BCMA binding domain, VHH1 CDR1 amino acid sequence
SHVMG

SEQ ID NO: 19 - protein, 17 aa, Lama glama
  source 1..17: note = Ciltacabtagene autoleucel CAR BCMA binding domain, VHH1 CDR2 amino acid sequence
VIGWRDISTSYADSVKG

SEQ ID NO: 20 - protein, 11 aa, Lama glama
  source 1..11: note = Ciltacabtagene autoleucel CAR BCMA binding domain, VHH1 CDR3 amino acid sequence
ARRIDAADFDS

SEQ ID NO: 21 - protein, 5 aa, Lama glama
  source 1..5: note = Ciltacabtagene autoleucel CAR BCMA binding domain, VHH2 CDR1 amino acid sequence
TFTMG

SEQ ID NO: 22 - protein, 17 aa, Lama glama
  source 1..17: note = Ciltacabtagene autoleucel CAR BCMA binding domain, VHH2 CDR2 amino acid sequence
AISLSPTLAYYAESVKG

SEQ ID NO: 23 - protein, 13 aa, Lama glama
  source 1..13: note = Ciltacabtagene autoleucel CAR BCMA binding domain, VHH2 CDR3 amino acid sequence
ADRKSVMSIRPDY

(19) United States
(12) Patent Application Publication
(10) Pub. No.: US 2023/0277589 A1
(43) Pub. Date: Sep. 7, 2023

(54) BCMA-TARGETED CAR-T CELL THERAPY FOR MULTIPLE MYELOMA

(71) Applicants: JANSSEN BIOTECH, INC., Horsham, PA (US); NANJING LEGEND BIOTECH CO., LTD., Nanjing (CN)

(72) Inventors: Muhammad AKRAM, Monroe, NJ (US); Kevin DEBRAGANCA, Chappaqua, NY (US); Xiaohu FAN, Edmonton (CA); Dong GENG, Somerset, NJ (US); Tonia NESHEIWAT, Somerset, NJ (US); Lida PACAUD, Somerset, NJ (US); Jordan SCHECTER, Raritan, NJ (US); Helen VARSOS, Spring House, PA (US); Enrique ZUDAIRE, Ambler, PA (US)

(73) Assignees: JANSSEN BIOTECH, INC., Horsham, PA (US); NANJING LEGEND BIOTECH CO., LTD., Nanjing (CN)

(21) Appl. No.: 18/052,349

(22) Filed: Nov. 3, 2022

Related U.S. Application Data
(60) Provisional application No. 63/275,471, filed on Nov. 4, 2021.

(30) Foreign Application Priority Data
Nov. 4, 2021 (WO) PCT/CN2021/128578

(51) Int. Cl. A61K 35/17; C07K 16/28; A61P 35/00; A61K 39/395; A61K 31/675; A61K 31/7076; A61K 31/167; A61K 31/138; C07K 14/705; A61K 38/17; C07K 14/725

(52) U.S. Cl. CPC A61K 35/17; A61K 31/138; A61K 31/167; A61K 31/675; A61K 31/7076; A61K 38/1774; A61K 39/3955; A61P 35/00; C07K 14/7051; C07K 14/70517; C07K 16/2866; C07K 16/2878; A61K 2039/5156

(57) ABSTRACT

Provided herein are methods of treating a subject who has multiple myeloma and has received prior treatment and has limited treatment options. Infusions of chimeric antigen receptor (CAR)-T cells comprising an anti-BCMA CAR comprising a polypeptide are administered to the subject. In certain embodiments, the dose of CAR-T cells administered to the subject is from 1.0 × 10⁵ to 5.0 × 10⁶ of CAR-T cells per kilogram of the subject’s mass. The method of treatment is effective in obtaining and maintaining minimal residual disease negativity status, as well as other beneficial clinical outcomes related to efficacy and safety.

### CROSS-REFERENCE TO RELATED APPLICATIONS

This application claims benefit of priority of International Patent Application No. PCT/CN2021/128578 filed on Nov. 4, 2021 and U.S. Provisional Pat. Application No. 63/275,471 filed on Nov. 4, 2021, the disclosure of each of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14651-048-999_SEQ_LISTING.xml”, was created on Oct. 27, 2022, and is 28,401 bytes in size.

### 1. BACKGROUND

Multiple myeloma is a neoplasm of plasma cells that is aggressive. Multiple myeloma is considered to be a B-cell neoplasm that proliferates uncontrollably in the bone marrow. Symptoms include one or more of hypercalcemia, renal insufficiency, anemia, bony lesions, bacterial infections, hyperviscosity and amyloidosis. Multiple myeloma is still considered to be an incurable disease, despite availability of new therapies that include proteasome inhibitors, immunomodulatory drugs, and monoclonal antibodies that have significantly improved patient outcomes. Because most patients will either relapse or become refractory to treatment, there is an ongoing need for new therapies for multiple myeloma.

### 2. SUMMARY OF THE DISCLOSURE

In one aspect is provided a method of treating a subject, said method comprising administering to the subject a composition comprising a therapeutically effective number of T cells comprising a chimeric antigen receptor (CAR) comprising:
(a) an extracellular antigen binding domain comprising:
   (1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
   (2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain,
to deliver to the subject a dose of CAR expressing T cells (CAR-T cells),
wherein said subject has multiple myeloma, has received prior treatment with one, two or three prior lines of therapy and is lenalidomide-refractory.

In some embodiments, the multiple myeloma is refractory to the last line of therapy. In some embodiments, the subject has relapsed after said one, two or three prior lines of therapy. In some embodiments, the subject received prior treatment with at least one prior line of therapy comprising treatment with lenalidomide and at least one non-lenalidomide medicament, said at least one non-lenalidomide medicament comprising at least one of a proteasomal inhibitor, an immunomodulatory drug or an anti-CD38 antibody. In some embodiments, the subject received prior treatment with at least two prior lines of therapy. In some embodiments, the subject received prior treatment with three prior lines of therapy.

In some embodiments, the subject received prior treatment with dexamethasone, an alkylating agent or daratumumab. In some embodiments, the multiple myeloma is refractory to three classes of medicaments.

In some embodiments, said method is effective in obtaining minimal residual disease (MRD) negative status in said subject assessed in the bone marrow after said administration of said CAR-T cells. In some embodiments, said minimal residual disease (MRD) negative status is obtained at a first follow-up time of between approximately 29 days and approximately 184 days after said administration of said CAR-T cells. In some embodiments, said method is effective in maintaining said minimal residual disease (MRD) negative status in said subject assessed in the bone marrow at a second follow-up time of between approximately 57 days and approximately 191 days after said administration of said CAR-T cells, further wherein said first follow-up time is earlier than said second follow-up time.

In some embodiments, said method is effective in obtaining said minimal residual disease (MRD) negative status at a rate of between approximately 24% and approximately 61% at a sensitivity threshold level of 10⁻⁴, 10⁻⁵ or 10⁻⁶. In some embodiments, said method is effective in obtaining said minimal residual disease (MRD) negative status at a rate of approximately 41% at a sensitivity threshold level of 10⁻⁴, 10⁻⁵ or 10⁻⁶.

In some embodiments, said method is effective in obtaining said minimal residual disease (MRD) negative status at a rate of between approximately 64% and approximately 99% in subjects with evaluable samples at a sensitivity threshold level of 10⁻⁵. In some embodiments, said method is effective in obtaining said minimal residual disease (MRD) negative status at a rate of approximately 92% in subjects with evaluable samples at a sensitivity threshold level of 10⁻⁵.

In some embodiments, said method is effective in obtaining at least one response in the subject after said administration of said CAR-T cells, wherein said at least one response comprises, in order from better to worse, a stringent complete response, a complete response, a very good partial response, a partial response or a minimal response.

In some embodiments, said method is effective in obtaining a first response before a time of between approximately 21 days and approximately 99 days after said administration of said CAR-T cells. In some embodiments, said method is effective in obtaining a first response before a time of between approximately 21 days and approximately 55 days after said administration of said CAR-T cells. In some embodiments, said method is effective in obtaining a first response before approximately 36 days after said administration of said CAR-T cells. In some embodiments, said method is effective in obtaining a first response before approximately 30 days after said administration of said CAR-T cells.

In some embodiments, said method is effective in obtaining a best response of any one of minimal response, partial response, very good partial response, complete response or stringent complete response. In some embodiments, said method is effective in obtaining said best response of any one of minimal response, partial response, very good partial response, complete response or stringent complete response at a rate of between approximately 52% and approximately 87%. In some embodiments, said method is effective in obtaining said best response of any one of minimal response, partial response, very good partial response, complete response or stringent complete response at a rate of approximately 72%.

In some embodiments, said method is effective in obtaining a best response of any one of partial response, very good partial response, complete response or stringent complete response. In some embodiments, said method is effective in obtaining said best response of any one of partial response, very good partial response, complete response or stringent complete response at a rate of between approximately 49% and approximately 84%. In some embodiments, said method is effective in obtaining said best response of any one of partial response, very good partial response, complete response or stringent complete response at a rate of approximately 69%.

In some embodiments, said method is effective in obtaining a best response of any one of very good partial response, complete response or stringent complete response. In some embodiments, said method is effective in obtaining said best response of any one of very good partial response, complete response or stringent complete response at a rate of between approximately 49% and approximately 84%. In some embodiments, said method is effective in obtaining said best response of any one of very good partial response, complete response or stringent complete response at a rate of approximately 69%.

In some embodiments, said method is effective in obtaining a best response of complete response or stringent complete response. In some embodiments, said method is effective in obtaining said best response of complete response or stringent complete response at a rate of between approximately 39% and approximately 76%. In some embodiments, said method is effective in obtaining said best response of complete response or stringent complete response at a rate of approximately 58%.

In some embodiments, said method is effective in obtaining a best response of stringent complete response. In some embodiments, said method is effective in obtaining said best response of stringent complete response at a rate of between approximately 33% and approximately 70%. In some embodiments, said method is effective in obtaining said best response of stringent complete response at a rate of approximately 52%.

In some embodiments, said method is effective in obtaining progression-free survival of the subject. In some embodiments, said method is effective in obtaining said progression-free survival of the subject at a time between said administration of said CAR-T cells and approximately 55 days after said administration of said CAR-T cells. In some embodiments, said method is effective in obtaining said progression-free survival of the subject at a time between said administration of said CAR-T cells and approximately 297 days after said administration of said CAR-T cells.

In some embodiments, said method is effective in obtaining said progression-free survival at a rate of between approximately 62% and approximately 95% at a follow-up time of approximately 6 months or approximately 9 months after said administration of said CAR-T cells. In some embodiments, said method is effective in obtaining said progression-free survival at a rate of approximately 86% at a follow-up time of approximately 6 months or approximately 9 months after said administration of said CAR-T cells.

In some embodiments, said method further comprises treating said subject for cytokine release syndrome more than approximately 3 days after said administration of said CAR-T cells. In some embodiments, said method is effective in obtaining a rate of recovery from said cytokine release syndrome of between approximately 1% and approximately 90% at a time of approximately 7 days after first observance of said cytokine release syndrome.

In some embodiments, said method is effective in obtaining a rate of immune-effector cell associated neurotoxicity of between approximately 20% and approximately 99%.

In some embodiments, said method is effective in obtaining said best response before a time of between approximately 27 days and approximately 237 days after said administration of said CAR-T cells. In some embodiments, said method is effective in obtaining said best response before a time of between approximately 46 days and approximately 172 days after said administration of said CAR-T cells. In some embodiments, said method is effective in obtaining said best response before approximately 109 days after said administration of said CAR-T cells. In some embodiments, said method is effective in obtaining said best response before approximately 87 days after said administration of said CAR-T cells.

In some embodiments, said method is effective in maintaining a response in the subject at a follow-up time between the time of said first response and approximately 270 days after said administration of said CAR-T cells. In some embodiments, said method is effective in maintaining a response at a rate of between approximately 70% and approximately 99% at a follow-up time of approximately 6 months after said administration of said CAR-T cells. In some embodiments, said method is effective in maintaining a response at a rate of approximately 95% at a follow-up time of approximately 6 months after said administration of said CAR-T cells. In some embodiments, said method is effective in maintaining a response at a rate of between approximately 7% and approximately 92% at a follow-up time of approximately 9 months after said administration of said CAR-T cells. In some embodiments, said method is effective in maintaining a response at a rate of approximately 63% at a follow-up time of approximately 9 months after said administration of said CAR-T cells.

In some embodiments, said method is further effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells. In some embodiments, said method is effective in obtaining either minimal residual disease (MRD) negative complete response or minimal residual disease (MRD) negative stringent complete response at a rate of between approximately 18% and approximately 54% at a follow-up time of approximately 291 days after said administration of said CAR-T cells. In some embodiments, said method is effective in obtaining either minimal residual disease (MRD) negative complete response or minimal residual disease (MRD) negative stringent complete response at a rate of approximately 35% at a follow-up time of approximately 291 days after said administration of said CAR-T cells.

In one aspect is provided a method of treating a subject, said method comprising administering to the subject a composition comprising a therapeutically effective number of T cells comprising a chimeric antigen receptor (CAR) comprising:
(a) an extracellular antigen binding domain comprising:
   (1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
   (2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain,
to deliver to the subject a dose of CAR expressing T cells (CAR-T cells),
wherein said subject has multiple myeloma, has had a prior early relapse and has received prior treatment with one prior line of therapy, said one prior line of therapy comprising treatment with at least two medicaments, said at least two medicaments comprising a proteasomal inhibitor and an immunomodulatory drug.

In some embodiments, the subject was additionally treated with an anti-CD38 antibody. In some embodiments, the multiple myeloma is refractory to at least one medicament.

In some embodiments, said method is effective in obtaining minimal residual disease (MRD) negative status in said subject assessed in the bone marrow after said administration of said CAR-T cells. In some embodiments, said minimal residual disease (MRD) negative status is assessed in the bone marrow at a first follow-up time at a first follow-up time of between approximately 35 days and approximately 58 days after said administration of said CAR-T cells. In some embodiments, said method is effective in maintaining said minimal residual disease (MRD) negative status in said subject assessed in the bone marrow at a second follow-up time of between approximately 78 days and approximately 359 days after said administration of said CAR-T cells, further wherein said first follow-up time is earlier than said second follow-up time.

In some embodiments, said method is effective in obtaining said minimal residual disease (MRD) negative status at a rate of between approximately 26% and approximately 74% at a sensitivity threshold level of 10⁻⁴ or 10⁻⁵ or at a rate of between approximately 17% and approximately 64% at a sensitivity threshold level of 10⁻⁶. In some embodiments, said method is effective in obtaining said minimal residual disease (MRD) negative status at a rate of approximately 50% at a sensitivity threshold level of 10⁻⁴ or 10⁻⁵ or at a rate of approximately 39% at a sensitivity threshold level of 10⁻⁶.

In some embodiments, said method is effective in obtaining said minimal residual disease (MRD) negative status at a rate of between approximately 66% and approximately 100% in subjects with evaluable samples at a sensitivity threshold level of 10⁻⁵. In some embodiments, said method is effective in obtaining said minimal residual disease (MRD) negative status at a rate of approximately 100% in subjects with evaluable samples at a sensitivity threshold level of 10⁻⁵.

In some embodiments, said method is effective in obtaining at least one response in the subject after said administration of said CAR-T cells, wherein said at least one response comprises, in order from better to worse, a stringent complete response, a complete response, a very good partial response, a partial response or a minimal response.

In some embodiments, said method is effective in obtaining a first response before a time of between approximately 27 days and approximately 78 days after said administration of said CAR-T cells. In some embodiments, said method is effective in obtaining a first response before a time of between approximately 27 days and approximately 47 days after said administration of said CAR-T cells. In some embodiments, n said method is effective in obtaining a first response before approximately 33 days after said administration of said CAR-T cells. In some embodiments, said method is effective in obtaining a first response before approximately 28 days after said administration of said CAR-T cells.

In some embodiments, said method is effective in obtaining a best response of any one of minimal response, partial response, very good partial response, complete response or stringent complete response. In some embodiments, said method is effective in obtaining said best response of any one of minimal response, partial response, very good partial response, complete response or stringent complete response at a rate of between approximately 65% and approximately 99%. In some embodiments, said method is effective in obtaining said best response of any one of minimal response, partial response, very good partial response, complete response or stringent complete response at a rate of approximately 89%.

In some embodiments, said method is effective in obtaining a best response of any one of partial response, very good partial response, complete response or stringent complete response. In some embodiments, said method is effective in obtaining said best response of any one of partial response, very good partial response, complete response or stringent complete response at a rate of between approximately 65% and approximately 99%. In some embodiments, said method is effective in obtaining said best response of any one of partial response, very good partial response, complete response or stringent complete response at a rate of approximately 89%.

In some embodiments, said method is effective in obtaining a best response of any one of very good partial response, complete response or stringent complete response. In some embodiments, said method is effective in obtaining said best response of any one of very good partial response, complete response or stringent complete response at a rate of between approximately 41% and approximately 87%. In some embodiments, said method is effective in obtaining said best response of any one of very good partial response, complete response or stringent complete response at a rate of approximately 67%.

In some embodiments, said method is effective in obtaining a best response of complete response or stringent complete response. In some embodiments, said method is effective in obtaining said best response of complete response or stringent complete response at a rate of between and approximately 10% and approximately 54%. In some embodiments, said method is effective in obtaining said best response of complete response or stringent complete response at a rate of approximately 28%.

In some embodiments, said method is effective in obtaining a best response of stringent complete response. In some embodiments, said method is effective in obtaining said best response of stringent complete response at a rate of between approximately 6% and approximately 48%. In some embodiments, said method is effective in obtaining said best response of stringent complete response at a rate of approximately 22%.

In some embodiments, said method is effective in obtaining progression-free survival of the subject. In some embodiments, said method is effective in obtaining said progression-free survival of the subject at a time between said administration of said CAR-T cells and approximately 182 days after said administration of said CAR-T cells.

In some embodiments, said method is effective in obtaining said progression-free survival at a rate of approximately 100% at a follow-up time of approximately 6 months after said administration of said CAR-T cells. In some embodiments, said method is effective in obtaining said progression-free survival at a rate of between approximately 5% and approximately 95% at a follow-up time of approximately 9 months or approximately 12 months after said administration of said CAR-T cells. In some embodiments, said method is effective in obtaining said progression-free survival at a rate of approximately 67% at a follow-up time of approximately 9 months or approximately 12 months after said administration of said CAR-T cells.

In some embodiments, said method further comprises treating said subject for cytokine release syndrome more than approximately 3 days after said administration of said CAR-T cells. In some embodiments, said method is effective obtaining a rate of recovery from said cytokine release syndrome of between approximately 1% and approximately 100% at a time of approximately 7 days after first observance of said cytokine release syndrome.

In some embodiments, said method is effective in obtaining said best response before a time of between approximately 27 days and approximately 354 days after said administration of said CAR-T cells. In some embodiments, said method is effective in obtaining said best response before a time of between approximately 27 days and approximately 155 days after said administration of said CAR-T cells. In some embodiments, said method is effective in obtaining said best response before approximately 71 days after said administration of said CAR-T cells. In some embodiments, said method is effective in obtaining said best response before approximately 42 days after said administration of said CAR-T cells.

In some embodiments, said method is effective in maintaining a response in the subject at a follow-up time of between the time of said first response and approximately 156 days after said administration of said CAR-T cells. In some embodiments, said method is effective in maintaining a response at a rate of between approximately 5% and approximately 95% at a follow-up time of approximately 6 months, approximately 9 months or approximately 12 months after said administration of said CAR-T cells. In some embodiments, said method is effective in maintaining a response at a rate of approximately 67% at a follow-up time of approximately 6 months, approximately 9 months or approximately 12 months after said administration of said CAR-T cells.

In some embodiments, said method is further effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells. In some embodiments, said method is effective in obtaining either minimal residual disease (MRD) negative complete response or minimal residual disease (MRD) negative stringent complete response at a rate of between approximately 1% and approximately 35% at a follow-up time of approximately 141 days after said administration of said CAR-T cells. In some embodiments, said method is effective in obtaining either minimal residual disease (MRD) negative complete response or minimal residual disease (MRD) negative stringent complete response at a rate of approximately 11% at a follow-up time of approximately 141 days after said administration of said CAR-T cells.

In one aspect is provided a method of treating a subject, said method comprising administering to the subject a composition comprising a therapeutically effective number of T cells comprising a chimeric antigen receptor (CAR) comprising:
(a) an extracellular antigen binding domain comprising:
   (1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
   (2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain,
to deliver to the subject a dose of CAR expressing T cells (CAR-T cells),
wherein said subject has multiple myeloma and has received at least one prior line of therapy comprising treatment with at least four medicaments, said at least four medicaments comprising a non-cellular BCMA-targeting medicament.

In some embodiments, said at least four medicaments further comprises a proteasomal inhibitor, an immunomodulatory drug and an anti-CD38 antibody.

In some embodiments, the subject received prior treatment with at least two prior lines of therapy. In some embodiments, the subject received prior treatment with at least four prior lines of therapy. In some embodiments, the subject received prior treatment with at least eight prior lines of therapy. In some embodiments, the subject received prior treatment with at least twelve prior lines of therapy. In some embodiments, the subject has relapsed after said at least one prior line of therapy.

In some embodiments, said method is effective in obtaining minimal residual disease (MRD) negative status in said subject assessed in the bone marrow after said administration of said CAR-T cells. In some embodiments, said minimal residual disease (MRD) negative status is assessed in the bone marrow at a first follow-up time at a first follow-up time of between approximately 56 days and approximately 58 days after said administration of said CAR-T cells. In some embodiments, said method is effective in maintaining said minimal residual disease (MRD) negative status in said subject assessed in the bone marrow at a second follow-up time of between approximately 183 days and approximately 186 days after said administration of said CAR-T cells, further wherein said first follow-up time is earlier than said second follow-up time.

In some embodiments, said method is effective in obtaining said minimal residual disease (MRD) negative status at a rate of between approximately 9% and approximately 49% at a sensitivity threshold level of 10⁻⁴, at a rate of between approximately 6% and approximately 44% at a sensitivity threshold level of 10⁻⁵, or at a rate of between approximately 1% and approximately 31% at a sensitivity threshold level of 10⁻⁶. In some embodiments, said method is effective in obtaining said minimal residual disease (MRD) negative status at a rate of approximately 25% at a sensitivity threshold level of 10⁻⁴, at a rate of approximately 20% at a sensitivity threshold level of 10⁻⁵, or at a rate of approximately 10% at a sensitivity threshold level of 10⁻⁶.

In some embodiments, said method is effective in obtaining said minimal residual disease (MRD) negative status at a rate of between approximately 22% and approximately 96% in subjects with evaluable samples at a sensitivity threshold level of 10⁻⁵. In some embodiments, said method is effective in obtaining said minimal residual disease (MRD) negative status at a rate of approximately 67% in subjects with evaluable samples at a sensitivity threshold level of 10⁻⁵.

In some embodiments, said method is effective in obtaining at least one response in the subject after said administration of said CAR-T cells, wherein said at least one response comprises, in order from better to worse, a stringent complete response, a complete response, a very good partial response, a partial response or a minimal response.

In some embodiments, said method is effective in obtaining a first response before a time of between approximately 27 days and approximately 153 days after said administration of said CAR-T cells. In some embodiments, said method is effective in obtaining a first response before a time of between approximately 27 days and approximately 88 days after said administration of said CAR-T cells. In some embodiments, said method is effective in obtaining a first response before approximately 43 days after said administration of said CAR-T cells. In some embodiments, said method is effective in obtaining a first response before approximately 28 days after said administration of said CAR-T cells.

In some embodiments, said method is effective in obtaining a best response of any one of minimal response, partial response, very good partial response, complete response or stringent complete response. In some embodiments, said method is effective in obtaining said best response of any one of minimal response, partial response, very good partial response, complete response or stringent complete response at a rate of between approximately 23% and approximately 69%. In some embodiments, said method is effective in obtaining said best response of any one of minimal response, partial response, very good partial response, complete response or stringent complete response at a rate of approximately 45%.

In some embodiments, said method is effective in obtaining a best response of any one of partial response, very good partial response, complete response or stringent complete response. In some embodiments, aid method is effective in obtaining said best response of any one of partial response, very good partial response, complete response or stringent complete response at a rate of between approximately 19% and approximately 64%. In some embodiments, said method is effective in obtaining said best response of any one of partial response, very good partial response, complete response or stringent complete response at a rate of approximately 40%.

In some embodiments, said method is effective in obtaining a best response of any one of very good partial response, complete response or stringent complete response. In some embodiments, said method is effective in obtaining said best response of any one of very good partial response, complete response or stringent complete response at a rate of between approximately 15% and approximately 59%. In some embodiments, said method is effective in obtaining said best response of any one of very good partial response, complete response or stringent complete response at a rate of approximately 35%.

In some embodiments, said method is effective in obtaining a best response of complete response or stringent complete response. In some embodiments, said method is effective in obtaining said best response of complete response or stringent complete response at a rate of between approximately 3% and approximately 38%.

In some embodiments, said method is effective in obtaining a best response of stringent complete response. In some embodiments, said method is effective in obtaining said best response of stringent complete response at a rate of between approximately 1% and approximately 32%.

In some embodiments, said method is effective in obtaining progression-free survival of the subject. In some embodiments, said method is effective in obtaining said progression-free survival of the subject at a time between said administration of said CAR-T cells and approximately 15 days after said administration of said CAR-T cells. In some embodiments, said method is effective in obtaining said progression-free survival of the subject at a time between said administration of said CAR-T cells and approximately 44 days after said administration of said CAR-T cells. In some embodiments, said method is effective in obtaining said progression-free survival of the subject at a time between said administration of said CAR-T cells and approximately 159 days after said administration of said CAR-T cells. In some embodiments, said method is effective in obtaining said progression-free survival at a rate of between approximately 29% and approximately 75% at a follow-up time of approximately 6 months or approximately 9 months after said administration of said CAR-T cells. In some embodiments, said method is effective in obtaining said progression-free survival at a rate of approximately 55% at a follow-up time of approximately 6 months or approximately 9 months after said administration of said CAR-T cells.

In some embodiments, said method is effective in obtaining a rate of cytokine release syndrome of between approximately 60% and approximately 99%. In some embodiments, said method further comprises treating said subject for cytokine release syndrome more than approximately 3 days after said administration of said CAR-T cells.

In some embodiments, said method is effective in obtaining a rate of immune-effector cell associated neurotoxicity of between approximately 20% and approximately 99%.

In some embodiments, said method is effective in obtaining said best response before a time of between approximately 27 days and approximately 171 days after said administration of said CAR-T cells. In some embodiments, said method is effective in obtaining said best response before a time of between approximately 27 days and approximately 133 days after said administration of said CAR-T cells. In some embodiments, said method is effective in obtaining said best response before approximately 78 days after said administration of said CAR-T cells. In some embodiments, said method is effective in obtaining said best response before approximately 56 days after said administration of said CAR-T cells.

In some embodiments, said method is effective in maintaining a response in the subject at a follow-up time of between the time of said first response and approximately 132 days after said administration of said CAR-T cells, further wherein said first response was obtained between the time of said administration of said CAR-T cells and approximately 131 days after said administration of said CAR-T cells. In some embodiments, said method is effective in maintaining a response at a rate of between approximately 20% and approximately 96% at a follow-up time of approximately 6 months after said administration of said CAR-T cells. In some embodiments, said method is effective in maintaining a response at a rate of approximately 80% at a follow-up time of approximately 6 months after said administration of said CAR-T cells.

In some embodiments, the multiple myeloma is refractory to at least two medicaments. In some embodiments, the multiple myeloma is refractory to at least three medicaments. In some embodiments, the multiple myeloma is refractory to at least four medicaments. In some embodiments, the multiple myeloma is refractory to at least five medicaments.

In some embodiments, the subject has bone marrow plasma cells of between approximately 10% and approximately 30% before said administration of said CAR-T cells.

In some embodiments, the dose comprises 1.0 × 10⁵ to 5.0 × 10⁶ of said CAR-T cells per kilogram of the mass of the subject. In some embodiments, the dose comprises 5.0 × 10⁵ to 1.0 × 10⁶ of said CAR-T cells per kilogram of the mass of the subject. In some embodiments, the dose comprises approximately 0.75 × 10⁶ of said CAR-T cells per kilogram of the mass of the subject. In some embodiments, the dose comprises less than 1.0 × 10⁸ of said CAR-T cells per subject.

In some embodiments, said administration of said CAR-T cells is via a single intravenous infusion. In some embodiments, said single intravenous infusion is administered using a single bag of said CAR-T cells. In some embodiments, said administration of said single bag of said CAR-T cells is completed between the time at which said single bag of CAR-T cells is thawed and three hours after said single bag of CAR-T cells is thawed. In some embodiments, said single intravenous administration is administered using two bags of said CAR-T cells. In some embodiments, said administration of each of said two bags of said CAR-T cells is completed between the time at which a first bag of said two bags of CAR-T cells is thawed and three hours after said first bag of CAR-T cells is thawed.

In some embodiments, a lymphodepleting regimen precedes said administration of CAR-T cells by approximately 5 days to approximately 7 days. In some embodiments, said lymphodepleting regimen is administered intravenously. In some embodiments, said lymphodepleting regimen comprises administration of cyclophosphamide or administration of fludarabine. In some embodiments, said cyclophosphamide is administered intravenously at 300 mg/m². In some embodiments, said fludarabine is administered intravenously at 30 mg/m². In some embodiments, a lymphodepleting regimen comprising cyclophosphamide administered intravenously at 300 mg/m² and fludarabine administered intravenously at 30 mg/m² precedes said administration of CAR-T cells by approximately 5 days to approximately 7 days.

In some embodiments, the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject. In some embodiments, the subject had an increase in tumor burden despite said bridging therapy. In some embodiments, the subject had an increase in tumor burden of approximately 25% or greater despite said bridging therapy.

In some embodiments, said method further comprises treating said subject for cytokine release syndrome more than approximately 3 days following said administration of said CAR-T cells without significantly reducing expansion of said CAR-T cells in vivo. In some embodiments, said treatment of cytokine release syndrome comprises administering an IL-6R inhibitor to the subject. In some embodiments, said IL-6R inhibitor is an antibody. In some embodiments, said antibody inhibits IL-6R by binding its extracellular domain. In some embodiments, said IL-6R inhibitor prevents the binding of IL-6 to IL-6R. In some embodiments, the IL-6R inhibitor is tocilizumab.

In some embodiments, the subject is treated with pre-administration medication comprising an antipyretic and an antihistamine up to approximately 1 hour before said administration of said CAR-T cells. In some embodiments, said antipyretic comprises either paracetamol or acetaminophen. In some embodiments, said antipyretic is administered to the subject either orally or intravenously. In some embodiments, said antipyretic is administered to the subject at a dosage of between 650 mg and 1000 mg. In some embodiments, said antihistamine comprises diphenhydramine. In some embodiments, said antihistamine is administered to the subject either orally or intravenously. In some embodiments, said antihistamine is administered at a dosage of between 25 mg and 50 mg, or its equivalent. In some embodiments, said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent.

In some embodiments, the composition comprising CAR-T cells administered to the subject further comprises an excipient selected from dimethylsulfoxide or dextran-40.

In some embodiments, the first BCMA binding moiety and/or the second BCMA binding moiety is an anti-BCMA VHH. In some embodiments, the first BCMA binding moiety is a first anti-BCMA VHH and the second BCMA binding moiety is a second anti-BCMA VHH. In some embodiments, the first BCMA binding moiety comprises the amino acid sequence of SEQ ID NO: 2. In some embodiments, the first BCMA binding moiety comprises a polypeptide encoded by the nucleic acid sequence of SEQ ID NO: 10. In some embodiments, the second BCMA binding moiety comprises the amino acid sequence of SEQ ID NO: 4. In some embodiments, the second BCMA binding moiety comprises a polypeptide encoded by the nucleic acid sequence of SEQ ID NO: 12.

In some embodiments, the first BCMA binding moiety and the second BCMA binding moiety are connected to each other via a peptide linker. In some embodiments, the peptide linker comprises the amino acid sequence of SEQ ID NO: 3. In some embodiments, the peptide linker comprises a polypeptide encoded by the nucleic acid sequence of SEQ ID NO: 11.

In some embodiments, the CAR polypeptide further comprises a signal peptide located at the N-terminus of the polypeptide. In some embodiments, the signal peptide is derived from CD8-alpha. In some embodiments, the signal peptide comprises the amino acid sequence of

SEQ ID NO: 1. In some embodiments, the signal peptide comprises a polypeptide encoded by the nucleic acid sequence of SEQ ID NO: 9.

In some embodiments, the transmembrane domain comprises the amino acid sequence of SEQ ID NO: 6. In some embodiments, the transmembrane domain comprises a polypeptide encoded by the nucleic acid sequence of SEQ ID NO: 14.

In some embodiments, the intracellular signaling domain comprises a primary intracellular signaling domain of an immune effector cell. In some embodiments, the intracellular signaling domain is derived from CD3ζ. In some embodiments, the intracellular signaling domain comprises at least one co-stimulatory signaling domains. In some embodiments, the intracellular signaling domain comprises the amino acid sequence of SEQ ID NO: 8. In some embodiments, the intracellular signaling domain comprises a polypeptide encoded by the nucleic acid sequence of SEQ ID NO: 16. In some embodiments, the intracellular signaling domain comprises an amino acid sequence of SEQ ID NO: 7. In some embodiments, the intracellular signaling domain comprises a polypeptide encoded by the nucleic acid sequence of SEQ ID NO: 15.

In some embodiments, the CAR polypeptide further comprises a hinge domain located between the C-terminus of the extracellular antigen binding domain and the N-terminus of the transmembrane domain. In some embodiments, the hinge domain comprises the amino acid sequence of SEQ ID NO: 5. In some embodiments, the hinge domain comprises a polypeptide encoded by the nucleic acid sequence of SEQ ID NO: 13.

In some embodiments, the CAR comprises the amino acid sequence of SEQ ID NO: 17.

In some embodiments, the T cells are autologous T cells. In some embodiments, the T cells are allogeneic T cells.

In some embodiments, the subject is human.

In some embodiments, the subject has had no prior exposure to a BCMA-targeting medicament.

In some embodiments, the multiple myeloma is progressive.

### 3. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the expression of BCMA antigen on the surface of GC, memory and plasmablast cells in the lymph node, long-lived plasma cells in the bone marrow LN and MALT, and on multiple myeloma cells. BAFF-R antigen is not expressed on plasmablast cells, long-lived plasma cells, or multiple myeloma cells. TACI is expressed on memory and plasmablast cells, long-lived plasma cells, and multiple myeloma cells. CD138 is expressed only on long-lived plasma cells and multiple myeloma cells.

FIG. 2 shows the design of the ciltacabtagene autoleucel CAR. Ciltacabtagene autoleucel comprises two VHH domains, as opposed to a single VL domain and a single VH domain found on various other CARs. Ciltacabtagene autoleucel comprises intracellular CD137 and human CD3 zeta domains.

FIG. 3 shows a schematic for preparing virus encoding ciltacabtagene autoleucel CAR, transduction of the virus into a T cell from the patient, and then preparation of CAR T cells expressing ciltacabtagene autoleucel.

FIG. 4 shows a schematic of study design for ciltacabtagene autoleucel CAR T-cells. The patient population includes those with relapsed or Refractory Multiple Myeloma, with 3 prior lines or double refractory to PI/IMiD and prior PI, IMiD, anti-CD38 exposure. A primary objective is safety and establishment of RP2D, such as studying incidence and severity of adverse events (Phase 1b). Another primary objective is efficacy: ORR - PR or better as defined by IMWG (Phase 2). The following are secondary objectives: Incidence and severity of adverse events (Phase 2), and any further efficacy characterization.

FIG. 5 shows the reduction in disease burden (representing the type of measurable disease, i.e., serum M-protein, urine M-protein, or the difference between involved and uninvolved free light chain (dFLC)) in Cohort A responders in the All Treated Analysis Set. “a” denotes Bence-Jones proteinuria at baseline with a transient response during bridging therapy; output represents dFLC value.

FIG. 6 shows the reduction in disease burden (representing the type of measurable disease, i.e., serum M-protein, urine M-protein, or the difference between involved and uninvolved free light chain (dFLC)) in Cohort B responders in the All Treated Analysis Set.

FIG. 7 shows the reduction in disease burden (representing the type of measurable disease, i.e., serum M-protein, urine M-protein, or the difference between involved and uninvolved free light chain (dFLC)) in Cohort C responders in the All Treated Analysis Set. “a” denotes Bence-Jones proteinuria at baseline with a transient response during bridging therapy; output represents dFLC value. “b” denotes non-estimable (NE) measurable disease type; output represents dFLC value.

FIG. 8 shows the assessment of response and duration of response in Cohort A responders in the All Treated Analysis Set.

FIG. 9 shows the assessment of response and duration of response in Cohort B responders in the All Treated Analysis Set.

FIG. 10 shows the assessment of response and duration of response in Cohort C responders in the All Treated Analysis Set.

FIG. 11 shows a Kaplan-Meier plot for the assessment of duration of response in Cohort A responders in the All Treated Analysis Set.

FIG. 12 shows a Kaplan-Meier plot for the assessment of duration of response in Cohort B responders in the All Treated Analysis Set.

FIG. 13 shows a Kaplan-Meier plot for the assessment of duration of response in Cohort A responders in the All Treated Analysis Set.

FIG. 14 shows a Kaplan-Meier plot for the assessment of progression-free survival in Cohort A responders in the All Treated Analysis Set.

FIG. 15 shows a Kaplan-Meier plot for the assessment of progression-free survival in Cohort B responders in the All Treated Analysis Set.

FIG. 16 shows a Kaplan-Meier plot for the assessment of progression-free survival in Cohort C responders in the All Treated Analysis Set.

### 4. DETAILED DESCRIPTION

The disclosure provides methods of treating patients with multiple myeloma with CAR-T cells. In certain embodiments, the methods relate to treating patients with multiple myeloma who received prior treatment and have an overall survival rate of no greater than about 50%. The disclosure also provides related nucleic acids, recombinant expression vectors, host cells, populations of cells, antibodies, or antigen binding portions thereof, and pharmaceutical compositions relating to the immune cells and CAR-expressing T cells of the disclosure . Dosage regimens and dosage forms are also provided.

Several aspects of the disclosure are described below, with reference to examples for illustrative purposes only. It should be understood that numerous specific details, relationships, and methods are set forth to provide a full understanding of the disclosure . One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure .

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### 4.1. Definitions

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “protein” or “polypeptide” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid; these terms may also refer to the nucleic acid sequence including the portion coding for any amino acids added as an artifact of cloning, including any amino acids coded for by linkers.

The term “antibody” includes monoclonal antibodies (including full length 4-chain antibodies or full length heavy-chain only antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)2, and Fv). The term “immunoglobulin” (Ig) is used interchangeably with “antibody” herein. Antibodies contemplated herein include single-domain antibodies, such as heavy chain only antibodies. The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotype (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

A “full length antibody” is comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FW). Each VH and VL is composed of three CDRs and four FW segments, arranged from amino-to-carboxy-terminus in the following order: FW1, CDR1, FW2, CDR2, FW3, CDR3 and FW4.

“Complementarity determining regions (CDR)” are antigen-binding sites in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, J. Exp. Med. 132:211-50, 1970; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, Mol. Biol. 196:901-17, 1987). The International ImMunoGeneTics (IMGT) database (http://www_imgt_org) provides a standardized numbering and definition of antigen-binding sites. The correspondence between CDRs, HVs and IMGT delineations is described in Lefranc et al., Dev. Comparat. Immunol. 27:55-77, 2003. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification. The framework regions (FW) are adjacent to and between the CDRs in both the VL (LFW1, LFW2, LFW3, LFW4) and VH (HFW1, HFW2, HFW3, HFW4).

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs”. Some VHHs may also be known as “Nanobodies”. A camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH”. VHH is thus a special type of VH.

The term “variable” refers to the fact that certain segments of the variable domains differ extensively in sequence among antibodies. The V domain (i.e., variable domain) mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a β-sheet configuration, connected by three HVRs, which form loops connecting, and in some cases forming part of, the β-sheet structure. The HVRs in each chain are held together in close proximity by the FR regions and contribute to the formation of the antigen binding site of antibodies (with the HVRs from the other chain, if the antibody is not a sdAb) (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The terms “fragment of an antibody”, “antibody fragment”, “functional fragment of an antibody”, and “antigen-binding portion” are used interchangeably herein to mean one or more fragments or portions of an antibody that retain the ability to specifically bind to an antigen (see, generally, Holliger et al., Nat. Biotech., 23(9): 1 126-1129 (2005)). The antigen recognition moiety of the CAR encoded by the nucleic acid sequence disclosed herein can contain any BCMA-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol, 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH -VL polypeptide chains to generate a dimeric molecule having two functional antigen binding sites. Antibody fragments are known in the art and are described in more detail in, e.g., U.S. Pat. Application Publication 2009/0093024 A1. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

As used herein, the terms “specifically binds”, “specifically recognizes”, or “specific for” refer to measurable and reproducible interactions such as binding between a target and an antigen binding protein (such as a CAR or a VHH), which is determinative of the presence of the target in the presence of a heterogeneous population of molecules including biological molecules.

The term “specificity” refers to selective recognition of an antigen binding protein (such as a CAR or a VHH) for a particular epitope of an antigen. Natural antibodies, for example, are monospecific. The term “multispecific” denotes that an antigen binding protein (such as a CAR or antibody) has two or more antigen-binding sites of which at least two bind different antigen-binding specificities. “Bispecific” as used herein denotes that an antigen binding protein (such as a CAR or antibody) has two different antigen-binding specificities.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

A “chimeric antigen receptor” or “CAR” is an artificially constructed hybrid protein or polypeptide containing the antigen binding domains of at least one antibody (or antibody fragment) linked to T-cell signaling domains. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, advantageously, CARs do not dimerize with endogenous T cell receptor (TCR) α- and β-chains. T cells expressing a CAR are referred to herein as CAR T cells, CAR-T cells or CAR modified T cells, and these terms are used interchangeably herein. The cell can be genetically modified to stably express at least one antigen-binding domain on its surface, conferring novel antigen specificity that is MHC independent. “BCMA CAR” refers to a CAR having an extracellular binding domain specific for BCMA. “Bi-epitope CAR” refers to a CAR having an extracellular binding domain specific for two different epitopes of an antigen, such as BCMA.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1-, as well as CD4+, CD4-, CD8+ and CD8- cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs are typically transcription factor Foxp3-positive CD4+T cells and can also include transcription factor Foxp3-negative regulatory T cells that are IL-10-producing CD4+T cells.

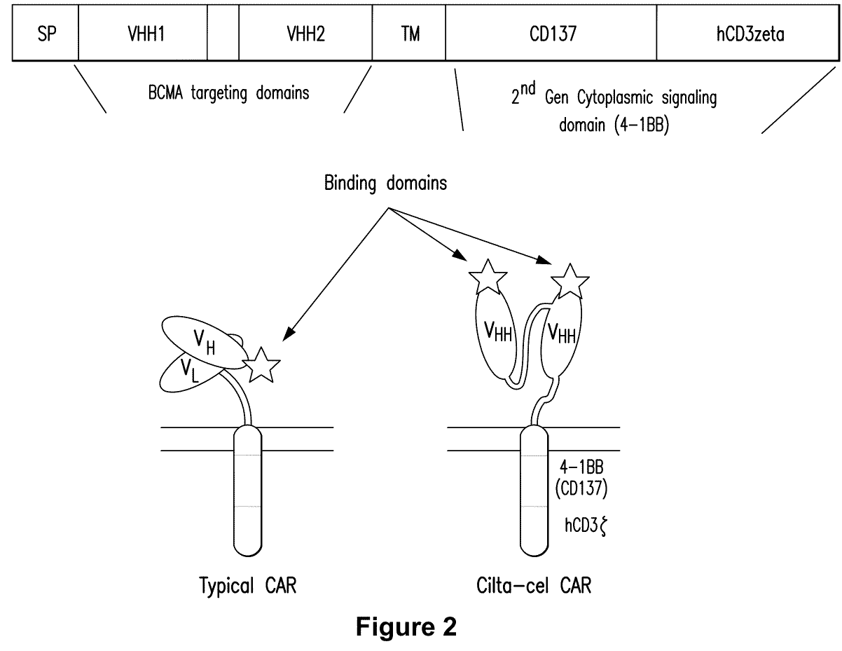

“Ciltacabtagene autoleucel” (“cilta-cel”) is a chimeric antigen receptor T cell (CAR-T) therapy comprising two B-cell maturation antigen (BCMA)-targeting VHH domains designed to confer avidity for BCMA. Cilta-cel can comprise T lymphocytes transduced with the ciltacabtagene autoleucel CAR, a CAR encoded by a lentiviral vector. The CAR targets the human B cell maturation antigen (anti-BCMA CAR). A diagram of the lentiviral vector encoding cilta-cel CAR is provided in FIG. 2. The amino acid sequence of the cilta-cel CAR is the amino acid sequence of SEQ ID NO: 17.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The terms “express” and “expression” mean allowing for or causing the information in a gene or DNA sequence to become produced. For example, expression can take the form of producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The terms “treat” or “treatment” refer to therapeutic treatment wherein the object is to slow down or lessen an undesired physiological change or disease, or provide a beneficial or desired clinical outcome during treatment. Beneficial or desired clinical outcomes include alleviation of symptoms, diminishment of extent of disease, stabilization (i.e., a cessation in the worsening) of the state of disease, delay or slowing of disease progression, amelioration or palliation of the disease state, and/or remission (whether partial or total and whether detectable or undetectable). “Treatment” can also mean prolonging survival as compared to expected survival if a subject was not receiving treatment. Those in need of treatment include those subjects already with the undesired physiological change or disease as well as those subjects prone to having the physiological change or disease. Treatment may involve a treatment agent, also referred to herein as a “medicament” or “medication,” that may be intended to help achieve the beneficial or desired clinical outcome of interest by its action. Treatment agents or medicaments may be administered to a subject by many routes, including at least intravenous and oral routes. The term “intravenous,” in connection to the administration of treatment agents or medicaments, refers to the administration of said treatment agents or medicaments within one or more veins. The term “oral,” in connection to the administration of treatment agents or medicaments, refers to the administration of said treatment agents or medicaments via an oral passage such as the mouth.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be of the order Carnivora, including felines (cats) and canines (dogs). The mammals may be of the order Artiodactyla, including bovines (cows) and swines (pigs) or of the order Perssodactyla, including equines (horses). The mammals may be of the order Primates, Ceboids, or Simoids (monkeys) or of the order Anthropoids (humans and apes). In some embodiments, the mammal is a human.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “line of therapy,” as used in connection with methods of treatment herein, refers to one or more cycles of a planned treatment program, which may have consisted of one or more planned cycles of single-agent therapy or combination therapy, as well as a sequence of treatments administered in a planned manner. For example, a planned treatment approach of induction therapy followed by autologous stem cell transplantation followed by maintenance is one line of therapy. A new line of therapy is considered to have started when a planned course of therapy has been modified to include other treatment agents or medicaments (alone or in combination) as a result of disease progression, relapse, or toxicity. A new line of therapy is also considered to have started when a planned period of observation off therapy had been interrupted by a need for additional treatment for the disease.

The term “refractory,” as used in connection to treatment with a particular treatment agent or medicament or line of therapy herein, refers to diseases or disease subjects that fail to respond to said treatment agent or medicament or line of therapy. The phrase “refractory myeloma” refers to multiple myeloma that is nonresponsive while on primary or salvage therapy or that has progressed within 60 days of last therapy.

The phrase “nonresponsive disease” refers to either failure to achieve minimal response or to development of progressive disease while on therapy.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30, or more times (e.g., 500, 1000 times) (including all integers and decimal points in-between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in-between and above 1, e.g., 1.5, 1.6, 1.7. 1.8, etc.) the response (reference response) produced by vehicle, a control composition, or the response in a particular cell lineage.

The terminology used herein is for the purpose of describing particular embodiments only and is not intended to be limiting. As used herein, the indefinite articles “a”, “an” and “the” should be understood to include plural reference unless the context clearly indicates otherwise.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99 % identity, includes something with 95 %, 96 %, 97 %, 98 % or 99 % identity, and includes subranges such as 96-99 %, 96-98 %, 96-97 %, 97-99 %, 97-98 % and 98-99 % identity. This applies regardless of the breadth of the range.

### 4.2. Vectors

Polynucleotide sequences encoding the CARs described in the present application can be obtained using standard recombinant techniques. Desired polynucleotide sequences may be isolated and sequenced from antibody producing cells such as hybridoma cells. Alternatively, polynucleotides can be synthesized using nucleotide synthesizers or PCR techniques.

The disclosure also provides a vector comprising the nucleic acid sequence encoding the CAR disclosed herein. The vector can be, for example, a plasmid, a cosmid, a viral vector (e.g., retroviral or adenoviral), or a phage. Suitable vectors and methods of vector preparation are well known in the art (see, e.g., Sambrook et al. and Ausubel et al.).

In addition to the nucleic acid sequence encoding the CAR disclosed herein, the vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

In some embodiments, the vector comprises a promoter. A large number of promoters recognized by a variety of potential host cells are well known. The selected promoter can be operably linked to cistron DNA encoding the CAR disclosed herein by removing the promoter from the source DNA via restriction enzyme digestion and inserting the isolated promoter sequence into the vector of the present application. A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pats. 5,464,758 and 5,814,618), the Ecdysone inducible system (No et al., Proc. Natl. Acad. Sci., 93: 3346-3351 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27: 4324- 4327 (1999); Nuc. Acid. Res., 28: e99 (2000); U.S. Pat. 7,112,715; and Kramer & Fussenegger, Methods Mol. Biol, 308: 123-144 (2005)).

In some embodiments, the vector comprises an “enhancer.” The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (for e.g., from depositories such as the ATCC as well as other commercial or individual sources). A number of polynucleotides comprising promoters (such as the commonly used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus. Such Ig enhancers include, for example, the heavy chain (mu) 5′ enhancers, light chain (kappa) 5′ enhancers, kappa and mu intronic enhancers, and 3′ enhancers (see generally Paul W.E. (ed), Fundamental Immunology, 3rd Edition, Raven Press, New York (1993), pages 353-363; and U.S. Pat. 5,885,827).

In some embodiments, the vector comprises a “selectable marker gene.” The term “selectable marker gene”, as used herein, refers to a nucleic acid sequence that allows cells expressing the nucleic acid sequence to be specifically selected for or against, in the presence of a corresponding selective agent. Suitable selectable marker genes are known in the art and described in, for e.g., International Patent Application Publications WO 1992/08796 and WO 1994/28143; Wigler et al., Proc. Natl. Acad. Sci. USA, 77: 3567 (1980); O′Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150: 1 (1981); Santerre et al., Gene, 30: 147 (1984); Kent et al., Science, 237: 901-903 (1987); Wigler et al., Cell, IP. 223 (1977); Szybalska & Szybalski, Proc. Natl. Acad. Sci. USA, 48: 2026 (1962); Lowy et al., Cell, 22: 817 (1980); and U.S. Pats. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pB -CMV from Stratagene (La Jolla, CA) represent non-limiting examples of an episomal vector that uses T-antigen and the SV40 origin of replication in lieu of EBNAl and oriP.

In some embodiments, the vector is an “integrating expression vector,” which may randomly integrate into the host cell’s DNA or may include a recombination site to enable recombination between the expression vector and a specific site in the host cell’s chromosomal DNA. Such integrating expression vectors may utilize the endogenous expression control sequences of the host cell’s chromosomes to effect expression of the desired protein. Examples of vectors that integrate in a site specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Invitrogen (Carlsbad, CA), and pCI or pFNI OA (ACT) FLEXI™ from Promega (Madison, WI).

In some embodiments, the vector is a viral vector. Representative viral expression vectors include, but are not limited to, the adenovirus-based vectors (e.g., the adenovirus-based Per.C6 system available from Crucell, Inc. (Leiden, The Netherlands)), lentivirus-based vectors (e.g., the lentiviral-based pLPl from Life Technologies (Carlsbad, CA)), and retrovirus-based vectors (e.g., the pFB-ERV plus pCFB-EGSH from Stratagene (La Jolla, CA)). In a preferred embodiment, the vector is a lentiviral vector.

In some embodiments, the vector comprising the nucleic acid encoding the CAR disclosed herein is introduced into a host cell that is capable of containing a heterologous nucleic acid. As used herein, The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. Preferably, the host cell is a cell that can contain the expression vector. In preferred embodiments, host cells are those that can be easily and reliably grown, have reasonably fast growth rates, have well characterized expression systems, and can be transformed or transfected easily and efficiently. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK 293 cells, and the like. In a preferred embodiment, the host cells are HEK 293 cells. In some embodiments, the HEK 293 cells are derived from the ATCC SD-3515 line. In some embodiments, the HEK 293 cells are derived from, the IU-VPF MCB line. In some embodiments, the HEK 293 cells are derived from the IU-VPF MWCB line. In some embodiments, the host cell can be a peripheral blood lymphocyte (PBL), a peripheral blood mononuclear cell (PBMC), or a natural killer (NK). Preferably, the host cell is a natural killer (NK) cell. More preferably, the host cell is a T-cell.

For purposes of amplifying or replicating the recombinant expression vector, the host cell may be a prokaryotic cell, e.g., a DH5α cell. For purposes of producing a virus from a viral expression vector, the host cell may be a eukaryotic cell, e.g., a HEK 293 cell. For purposes of producing a recombinant CAR, the host cell can be a mammalian cell. The mammalian host cell preferably is a human cell. The host cell can be of any cell type, can originate from any type of tissue, and can be of any developmental stage. Methods for selecting suitable mammalian host cells and methods for transformation, culture, amplification, screening, and purification of cells are known in the art.

In some embodiments, the disclosure provides an isolated host cell which expresses the nucleic acid sequence encoding the CAR described herein.

In some embodiments, the host cell is a T-cell. The T-cell of the disclosure can be any T-cell, such as a cultured T-cell, e.g., a primary T-cell, or a T-cell from a cultured T-cell line, or a T-cell obtained from a mammal. If obtained from a mammal, the T-cell can be obtained from numerous sources, including but not limited to blood, bone marrow, lymph node, the thymus, or other tissues or fluids. T-cells can also be enriched for or purified. The T-cell preferably is a human T-cell (e.g., isolated from a human). The T-cell can be of any developmental stage, including but not limited to, a CD4+/CD8+ double positive T-cell, a CD4+ helper T-cell, e.g., Th, and Th2 cells, a CD8+ T- cell (e.g., a cytotoxic T-cell), a tumor infiltrating cell, a memory T-cell, a naive T-cell, and the like. In one embodiment, the T-cell is a CD8+ T-cell or a CD4+ T-cell. T-cell lines are available from, e.g., the American Type Culture Collection (ATCC, Manassas, VA), and the German Collection of Microorganisms and Cell Cultures (DSMZ) and include, for example, Jurkat cells (ATCC TIB- 152), Sup-Tl cells (ATCC CRL-1942), RPMI 8402 cells (DSMZ ACC-290), Karpas 45 cells (DSMZ ACC-545), and derivatives thereof.

In some embodiments, the host cell is a natural killer (NK) cell. NK cells are a type of cytotoxic lymphocyte that plays a role in the innate immune system. NK cells are defined as large granular lymphocytes and constitute a third kind of cells differentiated from the common lymphoid progenitor which also gives rise to B and T lymphocytes (see, e.g., Immunobiology, 5th ed., Janeway et al., eds., Garland Publishing, New York, NY (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, VA) and include, for example, NK-92 cells (ATCC CRL-2407), NK92MI cells (ATCC CRL-2408), and derivatives thereof.

The nucleic acid sequence encoding a CAR disclosed herein may be introduced into a cell by “transfection”, “transformation”, or “transduction”. “Transfection”, “transformation”, or “transduction”, as used herein, refer to the introduction of one or more exogenous polynucleotides into a host cell by using physical or chemical methods.

The term “transformation” means the introduction of one or more exogenous polynucleotides into bacterial cells that have been made competent for transformation, for e.g., by use of dimethylsulfoxide, divalent cations such as calcium, or polyethylene glycol. Many transformation techniques are known in the art and include heat shock and electric shock.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell’s genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species. Many transfection techniques are known in the art and include, for example, calcium phosphate DNA co-precipitation (see, e.g., Murray E.J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

The term “transduction” means the introduction of a foreign nucleic acid into a cell using a viral vector. Phage or viral vectors can be introduced into host cells via transduction by infectious viral particles. Said infectious viral particles may be grown in suitable packaging cells, many of which are commercially available and known in the art.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

### 4.3. Antibodies and Derived Proteins

Suitable methods of making antibodies are known in the art. For instance, standard hybridoma methods are described in, e.g., Köhler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Pat. Application Publication No. 2002/0197266 A1).

Phage display can also be used to generate an antibody. In this regard, phage libraries encoding antigen-binding variable (V) domains of antibodies can be generated using standard molecular biology and recombinant DNA techniques (see, e.g., Sambrook et al., supra, and Ausubel et al., supra). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

The antibodies, polypeptides, and proteins of embodiments of the disclosure (including functional portions and functional variants) can be subject to post-translational modifications. They can be glycosylated, esterified, N-acylated, amidated, carboxylated, phosphorylated, esterified, cyclized via, e.g., a disulfide bridge, or converted into an acid addition salt. In some embodiments, they are dimerized or polymerized, or conjugated.

The antibodies, polypeptides, and/or proteins of embodiments of the disclosure (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; and Epitope Mapping, ed. Westwood et al., Oxford University Press, Oxford, United Kingdom, 2001. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, some of the antibodies, polypeptides, and proteins of the disclosure (including functional portions and functional variants thereof) can be isolated and/or purified from a source, such as a plant, a bacterium, an insect, a mammal, etc. Methods of isolation and purification are known in the art. Alternatively, the antibodies, polypeptides, and/or proteins described herein (including functional portions and functional variants thereof) can be commercially synthesized. In this respect, the antibodies, polypeptides, and proteins can be synthetic, recombinant, isolated, and/or purified.

### 4.4. Chimeric Antigen Receptors

International Patent Publication No. WO 2018/028647 is incorporated by reference herein in its entirety. U.S. Pat. Publication No. 2018/0230225 is incorporated by reference herein in its entirety. International Patent Application No. PCT/CN2020/133598 is incorporated by reference herein in its entirety.

The disclosure provides for methods of treating a subject with cells expressing a chimeric antigen receptor (CAR). The CAR comprises an extracellular antigen binding domain comprising one or more single-domain antibodies. In various embodiments, there is provided a CAR targeting BCMA (also referred herein as “BCMA CAR”) comprising a polypeptide comprising: (a) an extracellular antigen binding domain comprising an anti-BCMA binding moiety; (b) a transmembrane domain; and (c) an intracellular signaling domain. In some embodiments, the anti-BCMA binding moiety is camelid, chimeric, human, or humanized. In some embodiments, the intracellular signaling domain comprises a primary intracellular signaling domain of an immune effector cell (such as T cell). In some embodiments, the primary intracellular signaling domain is derived from CD4. In some embodiments, the primary intracellular signaling domain is derived from CD3-zeta. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In certain embodiments, the co-stimulatory signaling domain is derived from CD 137.

In some embodiments, the BCMA CAR further comprises a hinge domain (such as a CD8-alpha hinge domain) located between the C-terminus of the extracellular antigen binding domain and the N-terminus of the transmembrane domain. In some embodiments, the BCMA CAR further comprises a signal peptide (such as a CD8-alpha signal peptide) located at the N-terminus of the polypeptide. In some embodiments, the polypeptide comprises from the N-terminus to the C-terminus: a CD8-alpha signal peptide, the extracellular antigen-binding domain, a CD8-alpha hinge domain, a CD28 transmembrane domain, a first co-stimulatory signaling domain derived from CD28, a second co-stimulatory signaling domain derived from CD 137, and a primary intracellular signaling domain derived from CD4. In some embodiments, the polypeptide comprises from the N-terminus to the C-terminus: a CD8-alpha signal peptide, the extracellular antigen-binding domain, a CD8-alpha hinge domain, a CD8-alpha transmembrane domain, a second co-stimulatory signaling domain derived from CD137, and a primary intracellular signaling domain derived from CD3-zeta. In some embodiments, the BCMA CAR is monospecific. In some embodiments, the BCMA CAR is monovalent.

The present application also provides CARs that have two or more (including, but not limited to, any one of 2, 3, 4, 5, 6, or more) binding moieties that specifically bind to an antigen, such as BCMA. In some embodiments, one or more of the binding moieties are antigen binding fragments. In some embodiments, one or more of the binding moieties comprise single-domain antibodies. In some embodiments, one or more of the binding moieties comprise a VHH.

In some embodiments, the CAR is a multivalent (such as bivalent, trivalent, or of higher number of valencies) CAR comprising a polypeptide comprising: (a) an extracellular antigen binding domain comprising a plurality (such as at least about any one of 2, 3, 4, 5, 6, or more) of binding moieties specifically binding to an antigen (such as a tumor antigen); (b) a transmembrane domain; and (c) an intracellular signaling domain.

In some embodiments, the binding moieties, such as VHHs (including the plurality of VHHs, or the first VHH and/or the second VHH) are camelid, chimeric, human, or humanized. In some embodiments, the binding moieties or VHHs are connected to each other via peptide bonds or peptide linkers. In some embodiments, each peptide linker is no more than about 50 (such as no more than about any one of 35, 25, 20, 15, 10, or 5) amino acids long.

In some embodiments, the first BCMA binding moiety and/or the second BCMA binding moiety is an anti-BCMA VHH. In some embodiments, the first BCMA binding moiety is a first anti-BCMA VHH and the second BCMA binding moiety is a second anti-BCMA VHH.

In some embodiments, the first anti-BCMA binding moiety comprises a first complementarity determining region (CDR1) comprising the amino acid sequence of SEQ ID NO: 18. In some embodiments, the first anti-BCMA binding moiety comprises a second complementarity determining region (CDR2) comprising the amino acid sequence of SEQ ID NO: 19. In some embodiments, the first anti-BCMA binding moiety comprises a third complementarity determining region (CDR3) comprising the amino acid sequence of SEQ ID NO: 20. In some embodiments, the first BCMA binding moiety comprises the amino acid sequence of SEQ ID NO: 2. In some embodiments, the first BCMA binding moiety comprises a polypeptide encoded by the nucleic acid sequence of SEQ ID NO: 10. In some embodiments, the first anti-BCMA binding moiety comprises one or more of, or all of, SEQ ID NO: 18, SEQ ID NO: 19, SEQ ID NO: 20 and SEQ ID NO: 2. These sequences correspond to the sequences present in cilta-cel.

In some embodiments, the second BCMA binding moiety comprises a first complementarity determining region (CDR1) comprising the amino acid sequence of SEQ ID NO: 21. In some embodiments, the second BCMA binding moiety comprises a second complementarity determining region (CDR2) comprising the amino acid sequence of SEQ ID NO: 22. In some embodiments, the second BCMA binding moiety comprises a third complementarity determining region (CDR3) comprising the amino acid sequence of SEQ ID NO: 23. In some embodiments, the second BCMA binding moiety comprises the amino acid sequence of SEQ ID NO: 4. In some embodiments, the second BCMA binding moiety comprises a polypeptide encoded by the nucleic acid sequence of SEQ ID NO: 12. In some embodiments, the second anti-BCMA binding moiety comprises one or more of, or all of, SEQ ID NO: 21, SEQ ID NO: 22, SEQ ID NO: 23 and SEQ ID NO: 4. These sequences correspond to the sequences present in cilta-cel.

In some embodiments, the first BCMA binding moiety and the second BCMA binding moiety are connected to each other via a peptide linker. In some embodiments, the peptide linker comprises the amino acid sequence of SEQ ID NO: 3. In some embodiments, the peptide linker comprises a polypeptide encoded by the nucleic acid sequence of SEQ ID NO: 11.

In some embodiments, the CAR further comprises a hinge domain (such as a CD8-alpha hinge domain) located between the C-terminus of the extracellular antigen binding domain and the N-terminus of the transmembrane domain. In some embodiments, the CAR further comprises a signal peptide (such as a CD8-alpha signal peptide) located at the N-terminus of the polypeptide.

Without wishing to be bound by theory, the CARs that are multivalent, or those CARs comprising an extracellular antigen binding domain comprising a first BCMA binding moiety and a second BCMA binding moiety, may be specially suitable for targeting multimeric antigens via synergistic binding by the different antigen binding sites, or for enhancing binding affinity or avidity to the antigen. Improved avidity may allow for a substantial reduction in the dose of CAR-T cells needed to achieve a therapeutic effect, such as a dose ranging from 4.0 × 10⁴ to 1.0 × 10⁶ CAR-T cells per kilogram of the mass of the subject, or 3.0 × 10⁶ to 1.0 × 10⁸ total CAR-T expressing cells. Monovalent CARs, such as bb2121, may need to be dosed at 5 to 10 times these amounts to achieve a comparable effect. In various embodiments, reduced dosage ranges may provide for substantial reduction in cytokine release syndrome (CRS) and other potentially dangerous side-effects of CAR-T therapy.

The various binding moieties (e.g., an extracellular antigen binding domain comprising a first BCMA binding moiety and a second BCMA binding moiety) in the CARs described herein may be connected to each other via peptide linkers. The peptide linkers connecting different binding moieties (such as VHHs) may be the same or different. Different domains of the CARs may also be connected to each other via peptide linkers. In some embodiments, the binding moieties (such as VHHs) are directly connected to each other without any peptide linkers.

The peptide linker in the CARs described herein can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The CARs of the present application comprise a transmembrane domain that can be directly or indirectly connected to the extracellular antigen binding domain.

The CAR may comprise a T-cell activation moiety. The T-cell activation moiety can be any suitable moiety derived or obtained from any suitable molecule. In one embodiment, for example, the T-cell activation moiety comprises a transmembrane domain. The transmembrane domain can be any transmembrane domain derived or obtained from any molecule known in the art. For example, the transmembrane domain can be obtained or derived from a CD8α molecule or a CD28 molecule. Without wishing to be bound by theory, CD8 is a transmembrane glycoprotein that serves as a co-receptor for the T- cell receptor (TCR) and is expressed primarily on the surface of cytotoxic T-cells. The most common form of CD8 exists as a dimer composed of a CD8 alpha (CD8α) and CD8 beta (CD8β) chain. CD28 is expressed on T-cells and provides co-stimulatory signals required for T-cell activation. CD28 is the receptor for CD80 (B7.1) and CD86 (B7.2). In a preferred embodiment, the CD8α and CD28 are human.

In addition to the transmembrane domain, the T-cell activation moiety may further comprise an intracellular (i.e., cytoplasmic) T-cell signaling domain. The intercellular T- cell signaling domain can be obtained or derived from a CD28 molecule, a CD3 zeta (ζ) molecule or modified versions thereof, a human Fc receptor gamma (FcRy) chain, a CD27 molecule, an OX40 molecule, a 4-1BB molecule, or other intracellular signaling molecules known in the art. Without wishing to be bound by theory: (1) CD28 is a T-cell marker important in T-cell co- stimulation; (2) CD3ζ associates with TCRs to produce a signal and contains immunoreceptor tyrosine-based activation motifs (ITAMs); and (3) 4-1BB, also known as CD137, transmits a potent costimulatory signal to T-cells, promoting differentiation and enhancing long-term survival of T lymphocytes. In a preferred embodiment, the CD28, CD3 zeta, 4- IBB, OX40, and CD27 are human.

The T-cell activation domain of the CAR encoded by the nucleic acid sequence disclosed herein can comprise any one of aforementioned transmembrane domains and any one or more of the aforementioned intercellular T-cell signaling domains in any combination. For example, the nucleic acid sequence disclosed herein can encode a CAR comprising a CD28 transmembrane domain and intracellular T-cell signaling domains of CD28 and CD3 zeta. Alternatively, for example, the nucleic acid sequence disclosed herein can encode a CAR comprising a CD8α transmembrane domain and intracellular T-cell signaling domains of CD28, CD3 zeta, the Fc receptor gamma (FcRy) chain, and/or 4-1 BB.

In some embodiments, the CAR polypeptide further comprises a signal peptide located at the N-terminus of the polypeptide. In some embodiments, the signal peptide is derived from CD8-alpha (CD8α SP). In some embodiments, the signal peptide comprises the amino acid sequence of SEQ ID NO: 1. In some embodiments, the signal peptide comprises a polypeptide encoded by the nucleic acid sequence of SEQ ID NO: 9.

In some embodiments, the transmembrane domain comprises the amino acid sequence of SEQ ID NO: 6. In some embodiments, the transmembrane domain comprises a polypeptide encoded by the nucleic acid sequence of SEQ ID NO: 14.

In some embodiments, the intracellular signaling domain comprises a primary intracellular signaling domain of an immune effector cell. In some embodiments, the intracellular signaling domain is derived from CD3ζ. In some embodiments, the intracellular signaling domain comprises at least one co-stimulatory signaling domains. In some embodiments, the intracellular signaling domain comprises the amino acid sequence of SEQ ID NO: 8. In some embodiments, the intracellular signaling domain comprises a polypeptide encoded by the nucleic acid sequence of SEQ ID NO: 16. In some embodiments, the intracellular signaling domain comprises an amino acid sequence of SEQ ID NO: 7. In some embodiments, the intracellular signaling domain comprises a polypeptide encoded by the nucleic acid sequence of SEQ ID NO: 15.

In some embodiments, the CAR polypeptide further comprises a hinge domain located between the C-terminus of the extracellular antigen binding domain and the N-terminus of the transmembrane domain. In some embodiments, the hinge domain comprises the amino acid sequence of SEQ ID NO: 5. In some embodiments, the hinge domain comprises a polypeptide encoded by the nucleic acid sequence of SEQ ID NO: 13.

In some embodiments, the CAR comprises one or more of, or all of, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 18, SEQ ID NO: 19, SEQ ID NO: 20, SEQ ID NO: 21, SEQ ID NO: 22 and SEQ ID NO: 23. In one embodiments, the CAR comprises SEQ ID NO: 17. In some embodiments, the CAR comprises a polypeptide encoded by the nucleic acid sequence of one or more of, or all of, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

### 4.5. Immune Effector Cell Compositions

“Immune effector cells” are immune cells that can perform immune effector functions. In some embodiments, the immune effector cells express at least FcyRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils. In some embodiments, the immune effector cells are T cells. In some embodiments, the T cells are autologous T cells. In some embodiments, the T cells are allogeneic T cells. In some embodiments, the T cells are CD4+/CD8-, CD4-/CD8+, CD4+/CD8+, CD4-/CD8-, or combinations thereof. In some embodiments, the T cells produce IL-2, TFN, and/or TNF upon expressing the CAR and binding to the target cells, such as CD20+ or CD19+ tumor cells. In some embodiments, the CD8+ T cells lyse antigen-specific target cells upon expressing the CAR and binding to the target cells.

Biological methods for introducing the vector into an immune effector cell include the use of DNA and RNA vectors. Viral vectors have become the most widely used method for inserting genes into mammalian, e.g., human cells. Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

Provided herein are dosage forms comprising 3.0 × 10⁷ to 1.0 × 10⁸ CAR-T cells comprising a CAR comprising a polypeptide comprising: (a) an extracellular antigen binding domain comprising a first BCMA binding moiety specifically binding to a first epitope of BCMA, and a second BCMA binding moiety specifically binding to a second epitope of BCMA; (b) a transmembrane domain; and (c) an intracellular signaling domain, wherein the first epitope and the second epitope are different. In some embodiments, there are provided dosage forms comprising 3.0 × 10⁷ to 1.0 × 10⁸ engineered immune effector cells (such as T-cells) comprising a CAR comprising a polypeptide comprising: (a) an extracellular antigen binding domain comprising a first anti-BCMA VHH specifically binding to a first epitope of BCMA, and a second anti-BCMA VHH specifically binding to a second epitope of BCMA; (b) a transmembrane domain; and (c) an intracellular signaling domain, wherein the first epitope and the second epitope are different. In certain embodiments, the dosage form comprises 3.0 × 10⁷ to 4.0 × 10⁷ of the CAR-T cells. In certain embodiments, the dosage form comprises 3.5 × 10⁷ to 4.5 × 10⁷ of the CAR-T cells. In certain embodiments, the dosage form comprises 4.0 × 10⁷ to 5.0 × 10⁷ of the CAR-T cells. In certain embodiments, the dosage form comprises 4.5 × 10⁷ to 5.5 × 10⁷ of the CAR-T cells. In certain embodiments, the dosage form comprises 5.0 × 10⁷ to 6.0 × 10⁷ of the CAR-T cells. In certain embodiments, the dosage form comprises 5.5 × 10⁷ to 6.5 × 10⁷ of the CAR-T cells. In certain embodiments, the dosage form comprises 6.0 × 10⁷ to 7.0 × 10⁷ of the CAR-T cells. In certain embodiments, the dosage form comprises 6.5 × 10⁷ to 7.5 × 10⁷ of the CAR-T cells. In certain embodiments, the dosage form comprises 7.0 × 10⁷ to 8.0 × 10⁷ of the CAR-T cells. In certain embodiments, the dosage form comprises 7.5 × 10⁷ to 8.5 × 10⁷ of the CAR-T cells. In certain embodiments, the dosage form comprises 8.0 × 10⁷ to 9.0 × 10⁷ of the CAR-T cells. In certain embodiments, the dosage form comprises 8.5 × 10⁷ to 9.5 × 10⁷ of the CAR-T cells. In certain embodiments, the dosage form comprises 9.0 × 10⁷ to 1.0 × 10⁸ of the CAR-T cells.

In some embodiments, the cell population of the CAR-T dosage forms described herein comprise a T cell or population of T cells, e.g., at various stages of differentiation. Stages of T cell differentiation include naive T cells, stem central memory T cells, central memory T cells, effector memory T cells, and terminal effector T cells, from least to most differentiated. After antigen exposure, naive T cells proliferate and differentiate into memory T cells, e.g., stem central memory T cells and central memory T cells, which then differentiate into effector memory T cells. Upon receiving appropriate T cell receptor, costimulatory, and inflammatory signals, memory T cells further differentiate into terminal effector T cells. See, e.g., Restifo. Blood. 124.4(2014):476-77; and Joshi et al. J. Immunol. 180.3(2008):1309-15.

Naive T cells can have the following expression pattern of cell surface markers: CCR7+, CD62L+, CD45RO-, CD95-. Stem central memory T cells (Tscm) can have the following expression pattern of cell surface markers: CCR7+, CD62L+, CD45RO-, CD95+. Central memory T cells (Tcm) can have the following expression pattern of cell surface markers: CCR7+, CD62L+, CD45RO+, CD95+. Effector memory T cells (Tem) can have the following expression pattern of cell surface markers: CCR7-, CD62L-, CD45RO+, CD95+. Terminal effector T cells (Teff) can have the following expression pattern of cell surface markers: CCR7-, CD62L-, CD45RO-, CD95+. See, e.g., Gattinoni et al. Nat. Med. 17(2011):1290-7; and Flynn et al. Clin. Translat. Immunol. 3(2014):e20.

### 4.6. Pharmaceutical Compositions and Formulations

Further provided by the present application are pharmaceutical compositions comprising any one of the anti-BCMA antibodies of the disclosure, or any one of the engineered immune effector cells comprising any one of the CARs (such as BCMA CARs) as described herein, and a pharmaceutically acceptable carrier. Pharmaceutical compositions can be prepared by mixing any of the immune effector cells described herein, having the desired degree of purity, with optional pharmaceutically acceptable carriers, excipients or stabilizers (Remington’s Pharmaceutical Sciences 16th edition, Osol, A. Ed. (1980)), in the form of lyophilized formulations or aqueous solutions. In certain embodiments, a pharmaceutical composition of CAR-T cells further comprises an excipient selected from dimethylsulfoxide or dextran-40.

The compositions described herein may be administered as part of a pharmaceutical composition comprising one or more carriers. The choice of carrier will be determined in part by the particular nucleic acid sequence, vector, or host cells expressing the CAR disclosed herein, as well as by the particular method used to administer the nucleic acid sequence, vector, or host cells expressing the CAR disclosed herein. Accordingly, there are a variety of suitable formulations of the pharmaceutical composition of the disclosure.

For example, the pharmaceutical composition can contain preservatives. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

In addition, buffering agents may be used in the composition. Suitable buffering agents include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001 % to about 4% by weight of the total composition.

The composition comprising the nucleic acid sequence encoding the CAR disclosed herein, or host cells expressing the CAR disclosed herein, can be formulated as an inclusion complex, such as cyclodextrin inclusion complex, or as a liposome. Liposomes can serve to target the host cells (e.g., T-cells or NK cells) or the nucleic acid sequence disclosed herein to a particular tissue. Liposomes also can be used to increase the half-life of the nucleic acid sequence disclosed herein. Many methods are available for preparing liposomes, such as those described in, for example, Szoka et al., Ann. Rev. Biophys. Bioeng., 9: 467 (1980), and U.S. Pats. 4,235,871; 4,501,728; 4,837,028; and 5,019,369. The composition can employ time-released, delayed release, and sustained release delivery systems such that the delivery of the composition disclosed herein occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain composition embodiments of the disclosure.

In certain embodiments, the CAR-T cells are formulated at a dose of about 1.0 x 10⁵ to 2.0 × 10⁵ cells/kg, 1.5 × 10⁵ to 2.5 × 10⁵ cells/kg, 2.0 × 10⁵ to 3.0 × 10⁵ cells/kg, 2.5 x 10⁵ to 3.5 × 10⁵ cells/kg, 3.0 × 10⁵ to 4.0 × 10⁵ cells/kg, 3.5 × 10⁵ to 4.5 × 10⁵ cells/kg, 4.0 x 10⁵ to 5.0 × 10⁵ cells/kg, 4.5 × 10⁵ to 5.5 × 10⁵ cells/kg, 5.0 × 10⁵ to 6.0 × 10⁵ cells/kg, 5.5 x 10⁵ to 6.5 × 10⁵ cells/kg, 6.0 × 10⁵ to 7.0 × 10⁵ cells/kg, 6.5 × 10⁵ to 7.5 × 10⁵ cells/kg, 7.0 x 10⁵ to 8.0 × 10⁵ cells/kg, 7.5 × 10⁵ to 8.5 × 10⁵ cells/kg, 8.0 × 10⁵ to 9.0 × 10⁵ cells/kg, 8.5 x 10⁵ to 9.5 × 10⁵ cells/kg, 9.0 × 10⁵ to 1.0 × 10⁶ cells/kg. In a preferred embodiment, the dose is formulated at approximately 0.75 × 10⁶ cells/kg. In certain embodiments, the CAR-T cells are formulated at a dose of less than 1.0 × 10⁸ cells per subject.

### 4.7. Methods of Treating Subjects

The present application further relates to methods and compositions for use in cell immunotherapy. In some embodiments, the cell immunotherapy is for treating cancer in a subject, including but not limited to hematological malignancies and solid tumors. In some embodiments, the subject is human. In some embodiments, the methods are suitable for treatment of adults and pediatric population, including all subsets of age, and can be used as any line of treatment, including first line or subsequent lines.

Any of the anti-BCMA VHHs, CARs, and engineered immune effector cells (such as CAR-T cells) described herein may be used in the method of treating cancer. In some embodiments, the immune effector cells are autologous. In some embodiments, the immune effector cells are allogeneic.

In certain embodiments, the CAR-T cells are administered at a dose of about 1.0 x 10⁵ to 2.0 × 10⁵ cells/kg, 1.5 × 10⁵ to 2.5 × 10⁵ cells/kg, 2.0 × 10⁵ to 3. × 10⁵ cells/kg, 2.5 × 10⁵ to 3.5 × 10⁵ cells/kg, 3.0 × 10⁵ to 4.0 × 10⁵ cells/kg, 3.5 × 10⁵ to 4.5 × 10⁵ cells/kg, 4.0 × 10⁵ to 5.0 × 10⁵ cells/kg, 4.5 × 10⁵ to 5.5 × 10⁵ cells/kg, 5.0 × 10⁵ to 6.0 × 10⁵ cells/kg, 5.5 × 10⁵ to 6.5 × 10⁵ cells/kg, 6.0 × 10⁵ to 7.0 × 10⁵ cells/kg, 6.5 × 10⁵ to 7.5 × 10⁵ cells/kg, 7.0 × 10⁵ to 8.0 × 10⁵ cells/kg, 7.5 × 10⁵ to 8.5 × 10⁵ cells/kg, 8.0 × 10⁵ to 9.0 × 10⁵ cells/kg, 8.5 × 10⁵ to 9.5 × 10⁵ cells/kg, 9.0 × 10⁵ to 1.0 × 10⁶ cells/kg, 1.0 × 10⁶ to 2.0 × 10⁶ cells/kg, 1.5 × 10⁶ to 2.5 × 10⁶ cells/kg, 2.0 × 10⁶ to 3.0 × 10⁶ cells/kg, 2.5 × 10⁶ to 3.5 × 10⁶ cells/kg, 3.0 × 10⁶ to 4.0 × 10⁶ cells/kg, 3.5 × 10⁶ to 4.5 × 10⁶ cells/kg, 4.0 × 10⁶ to 5.0 × 10⁶ cells/kg, 4.5 × 10⁶ to 5.5 × 10⁶ cells/kg, or 5.0 × 10⁶to 6.0 × 10⁶ cells/kg. In a preferred embodiment, the dose comprises approximately 0.75 × 10⁶ cells/kg. In certain embodiments, the CAR-T cells are administered at a dose of about 1.0 × 10⁸ cells per subject.

In certain embodiments, the CAR-T cells are administered at a dose of less than 1.0 × 10⁸ cells per subject. In certain embodiments, the CAR-T cells are administered at a dose of about 3.0 to 4.0 × 10⁷ cells. In certain embodiments, the CAR-T cells are administered at a dose of about 3.5 to 4.5 × 10⁷ cells. In certain embodiments, the CAR-T cells are administered at a dose of about 4.0 to 5.0 × 10⁷ cells. In certain embodiments, the CAR-T cells are administered at a dose of about 4.5 to 5.5 × 10⁷ cells. In certain embodiments, the CAR-T cells are administered at a dose of about 5.0 to 6.0 × 10⁷ cells. In certain embodiments, the CAR-T cells are administered at a dose of about 5.5 to 6.5 × 10⁷ cells. In certain embodiments, the CAR-T cells are administered at a dose of about 6.0 to 7.0 × 10⁷ cells. In certain embodiments, the CAR-T cells are administered at a dose of about 6.5 to 7.5 × 10⁷ cells. In certain embodiments, the CAR-T cells are administered at a dose of about 7.0 to 8.0 × 10⁷ cells. In certain embodiments, the CAR-T cells are administered at a dose of about 7.5 to 8.5 × 10⁷ cells. In certain embodiments, the CAR-T cells are administered at a dose of about 8.0 to 9.0 × 10⁷ cells. In certain embodiments, the CAR-T cells are administered at a dose of about 8.5 to 9.5 × 10⁷ cells. In certain embodiments, the CAR-T cells are administered at a dose of about 9.0 × 10⁷ to 1.0 × 10⁸ cells.

In certain embodiments, the CAR-T cells are administered at a dose of about 0.693 × 10⁶ CAR-positive viable T-cells/kg. In certain embodiments, the CAR-T cells are administered at a dose of about 0.52 × 10⁶ CAR-positive viable T-cells/kg. In certain embodiments, the CAR-T cells are administered at a dose of about 0.94 × 10⁶ CAR-positive viable T-cells/kg. In certain embodiments, the CAR-T cells are administered at a dose of about 0.709 × 10⁶ CAR-positive viable T-cells/kg. In certain embodiments, the CAR-T cells are administered at a dose of about 0.51 × 10⁶ CAR-positive viable T-cells/kg. In certain embodiments, the CAR-T cells are administered at a dose of about 0.95 × 10⁶ CAR-positive viable T-cells/kg. In certain embodiments, the CAR-T cells are administered in an outpatient setting.

In certain embodiments, the CAR-T cells (e.g., at any of the foregoing doses) are administered in one or more intravenous infusions. In certain embodiments, said administration of said CAR-T cells is via a single intravenous infusion. In certain embodiments, said single intravenous infusion is administered using a single bag of said CAR-T cells. In certain embodiments, said administration of said single bag of said CAR-T cells is completed between the time at which said single bag of CAR-T cells is thawed and three hours after said single bag of CAR-T cells is thawed. In certain embodiments, single intravenous administration is administered using two bags of said CAR-T cells. In certain embodiments, said administration of each of said two bags of said CAR-T cells is completed between the time at which a first bag of said two bags of CAR-T cells is thawed and three hours after said first bag of CAR-T cells is thawed.

In certain embodiments, the time since the initial apheresis to the administration of CAR-T cells is less than 41, 47, 54, 61, 68, 75, 82, 89, 96, 103, 110, 117, 124, 131, 138, 145, 152, 159, 166 or 167 days. In certain embodiments, the time since the initial apheresis to the administration of CAR-T cells is greater than 41, 47, 54, 61, 68, 75, 82, 89, 96, 103, 110, 117, 124, 131, 138, 145, 152, 159, 166 or 167 days.

The composition comprising the host cells expressing the CAR-encoding nucleic acid sequence disclosed herein, or a vector comprising the CAR-encoding nucleic acid sequence disclosed herein, can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral”, as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection. Most preferably, the composition is administered by intravenous infusion.

The composition comprising the host cells expressing the CAR-encoding nucleic acid sequence disclosed herein, or a vector comprising the CAR-encoding nucleic acid sequence disclosed herein, can be administered with one or more additional therapeutic agents, which can be coadministered to the mammal. By “coadministering” is meant administering one or more additional therapeutic agents and the composition comprising the host cells disclosed herein or the vector disclosed herein sufficiently close in time such that the CAR disclosed herein can enhance the effect of one or more additional therapeutic agents, or vice versa. In this regard, the composition comprising the host cells disclosed herein or the vector disclosed herein can be administered first, and the one or more additional therapeutic agents can be administered second, or vice versa.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In certain embodiments, a lymphodepleting regimen precedes said administration of CAR-T cells by approximately 5 days to approximately 7 days. In certain embodiments, lymphodepleting regimen is administered intravenously. In certain embodiments, said lymphodepleting regimen comprises administration of cyclophosphamide or administration of fludarabine. In certain embodiments, said cyclophosphamide is administered intravenously at 300 mg/m². In certain embodiments, said fludarabine is administered intravenously at 30 mg/m². In certain embodiments, a lymphodepleting regimen comprising cyclophosphamide administered intravenously at 300 mg/m² and fludarabine administered intravenously at 30 mg/m² precedes said administration of CAR-T cells by approximately 5 days to approximately 7 days.

In certain embodiments, the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject. In certain embodiments, the subject had an increase in tumor burden despite said bridging therapy. In certain embodiments, the subject had an increase in tumor burden of approximately 25% or greater despite said bridging therapy.

In certain embodiments, the subject is treated with pre-administration medication comprising an antipyretic and an antihistamine up to approximately 1 hour before said administration of said CAR-T cells. In certain embodiments, said antipyretic comprises either paracetamol or acetaminophen. In certain embodiments, said antipyretic is administered to the subject either orally or intravenously. In certain embodiments, said antipyretic is administered to the subject at a dosage of between 650 mg and 1000 mg. In certain embodiments, said antihistamine comprises diphenhydramine. In certain embodiments, said antihistamine is administered to the subject either orally or intravenously. In certain embodiments, said antihistamine is administered at a dosage of between 25 mg and 50 mg, or its equivalent. In certain embodiments, said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent.

In some embodiments, the method further comprises diagnosing said subject for cytokine release syndrome (CRS). In preferred embodiments, the diagnosis is made according to the American Society of Transplantation and Cellular Therapy (ASTCT), formerly the American Society for Blood and Marrow Transplantation (ASBMT) consensus grading. A non-limiting summary of the ASTCT consensus grading for CRS diagnosis is provided in Table 30. In some embodiments, the CRS is assessed by evaluating the levels of one or more of, or all of, IL-6, IL-10, IFN-γ, C-reactive protein (CRP) and ferritin.

In some embodiments, the method further comprises treating said subject for cytokine release syndrome (CRS). In some embodiments, the treatment of CRS is with an antipyretic. In some examples, the treatment of CRS is with anticytokine therapy. In some embodiments, the treatment of CRS occurs more than approximately 3 days following the infusion. In some embodiments, the treatment of CRS occurs without significantly reducing CAR-T cell expansion in vivo. In certain embodiments, said method further comprises treating said subject for cytokine release syndrome more than approximately 3 days following said administration of said CAR-T cells without significantly reducing expansion of said CAR-T cells in vivo. In some embodiments, the treatment of CRS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the anticytokine therapy comprises administration of tocilizumab. In some embodiments, the anticytokine therapy comprises administration of steroids. In some embodiments, treatment for CRS comprises treatment with monoclonal antibodies other than tocilizumab. In some embodiments, the antibodies other than tocilizumab target cytokines. In some embodiments, the cytokine that the antibodies other than tocilizumab target is IL-1. In some embodiments, the IL-1 targeting antibody is Anakinra. In some embodiments, the cytokine that the antibodies other than tocilizumab target is TNFα. In some embodiments, the treatment of CRS comprises administering to the subject a corticosteroid. In some embodiments, the treatment of CRS comprises using a vasopressor. In some embodiments, the treatment of CRS comprises intubation or mechanical ventilation. In some embodiments, the treatment of CRS comprises administering to the subject cyclophosphamide. In some embodiments, the treatment of CRS comprises administering to the subject etanercept. In some embodiments, the treatment of CRS comprises administering to the subject levetiracetam. In some embodiments, the treatment of CRS comprises supportive care.

In some embodiments, the method further comprises diagnosing said subject for immune cell effector-associated neurotoxicity (ICANS). In some embodiments, the diagnosis is made according to the National Cancer Institute Common Terminology Criteria for Adverse Events (NCI CTCAE) criteria. In some embodiments, the diagnosis is made according to the NCI CTCAE criteria, Version 5.0. In some embodiments, the diagnosis is made according to the American Society of Transplantation and Cellular Therapy (ASTCT) consensus grading system. In some embodiments, the embodiments, there is neurotoxicity consistent with ICAN. A non-limiting summary of the ASTCT consensus grading system for ICANS diagnosis is provided in Table 31. In some embodiments, the treatment of ICANS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the treatment of ICANS comprises administering to the subject an IL-1 inhibitor. In some embodiments the IL-1 inhibitor is an antibody. In a preferred embodiment, the IL-1 inhibiting antibody is Anakinra. In some embodiments, the treatment of ICANS comprises administering to the subject a corticosteroid. In some embodiments, the treatment of ICANS comprises administering to the subject levetiracetam. In some embodiments, the treatment of ICANS comprises administering to the subject dexamethasone. In some embodiments, the treatment of ICANS comprises administering to the subject methylprednisone sodium succinate. In some embodiments, the treatment of ICANS comprises administering to the subject pethidine. In some embodiments, the treatment of ICANS comprises administering to the subject one or more of, or all of, tocilizumab, Anakinra, a corticosteroid, levetiracetam, dexamethasone, methylprednisone sodium succinate or pethidine.

In some embodiments, the method further comprises diagnosing said subject for cytopenias. In some embodiments, the cytopenias comprise one or more of, or all of, lymphopenia, neutropenia, and thrombocytopenia. Without being bound by theory, a Grade 3 or Grade 4 but not a Grade 2 or lower lymphopenia is characterized by to a lymphocyte count less than 0.5×10⁹ cells per liter of a subject’s blood sample, a Grade 3 or Grade 4 but not a Grade 2 or lower neutropenia is characterized by a neutrophil count less than 1000 cells per microliter of a subject’s blood sample, and a Grade 3 or Grade 4 but not a Grade 2 or lower thrombocytopenia is characterized by a platelet count less than 50,000 cells per microliter of a subject’s blood sample. In some embodiments, greater than 75% subjects with Grade 3 or Grade 4 lymphopenia following CAR-T cell administration recover to Grade 2 or lower lymphopenia 60 days following CAR-T cell administration. In some embodiments, greater than 80% subjects with Grade 3 or Grade 4 lymphopenia following CAR-T cell administration recover to Grade 2 or lower lymphopenia 60 days following CAR-T cell administration. In some embodiments, greater than 85% subjects with Grade 3 or Grade 4 lymphopenia following CAR-T cell administration recover to Grade 2 or lower lymphopenia 60 days following CAR-T cell administration. In some embodiments, greater than 90% subjects with Grade 3 or Grade 4 lymphopenia following CAR-T cell administration recover to Grade 2 or lower lymphopenia 60 days following CAR-T cell administration. In some embodiments, greater than 70% subjects with Grade 3 or Grade 4 neutropenia following CAR-T cell administration recover to Grade 2 or lower neutropenia 60 days following CAR-T cell administration. In some embodiments, greater than 75% subjects with Grade 3 or Grade 4 neutropenia following CAR-T cell administration recover to Grade 2 or lower neutropenia 60 days following CAR-T cell administration. In some embodiments, greater than 80% subjects with Grade 3 or Grade 4 neutropenia following CAR-T cell administration recover to Grade 2 or lower neutropenia 60 days following CAR-T cell administration. In some embodiments, greater than 85% subjects with Grade 3 or Grade 4 neutropenia following CAR-T cell administration recover to Grade 2 or lower neutropenia 60 days following CAR-T cell administration. In some embodiments, greater than 30% subjects with Grade 3 or Grade 4 thrombocytopenia following CAR-T cell administration recover to Grade 2 or lower thrombocytopenia 60 days following CAR-T cell administration. In some embodiments, greater than 34% subjects with Grade 3 or Grade 4 thrombocytopenia following CAR-T cell administration recover to Grade 2 or lower thrombocytopenia 60 days following CAR-T cell administration. In some embodiments, greater than 38% subjects with Grade 3 or Grade 4 thrombocytopenia following CAR-T cell administration recover to Grade 2 or lower thrombocytopenia 60 days following CAR-T cell administration. In some embodiments, greater than 42% subjects with Grade 3 or Grade 4 thrombocytopenia following CAR-T cell administration recover to Grade 2 or lower thrombocytopenia 60 days following CAR-T cell administration.

Once the composition comprising host cells expressing the CAR-encoding nucleic acid sequence disclosed herein, or a vector comprising the CAR-encoding nucleic acid sequence disclosed herein, is administered to a mammal (e.g., a human), the biological activity of the CAR can be measured by any suitable method known in the art. In accordance with the method disclosed herein, the CAR binds to BCMA on the multiple myeloma cells, and the multiple myeloma cells are destroyed. Binding of the CAR to BCMA on the surface of multiple myeloma cells can be assayed using any suitable method known in the art, including, for example, ELISA and flow cytometry. The ability of the CAR to destroy multiple myeloma cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). The biological activity of the CAR also can be measured by assaying expression of certain cytokines, such as CD 107a, IFNγ, IL-2, and TNF.

The methods described herein may be used for treating various cancers, including both solid cancer and liquid cancer. In certain embodiments, the methods are used to treat multiple myeloma. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

In certain embodiments, the cancer is multiple myeloma. In certain embodiments, the cancer is stage I, stage II or stage III, and/or stage A or stage B multiple myeloma based on the Durie-Salmon staging system. In certain embodiments, the cancer is stage I, stage II or stage III multiple myeloma based on the International staging system published by the International Myeloma Working Group (IMWG). In some embodiments, the multiple myeloma is progressive.

In certain embodiments, the subject received prior treatment with at least one prior line of therapy. In certain embodiments, the at least one prior line of therapy comprises treatment with a medicament that is a proteasomal inhibitor (PI). Non-limiting examples of a PI include bortezomib, carfilzomib and ixazomib. In certain embodiments, the at least one prior line of therapy comprises treatment with a medicament that is an immunomodulatory drug (IMiD). Non-limiting examples of an IMiD include lenalidomide, pomalidomide and thalidomide. In certain embodiments, the at least one prior line of therapy comprises treatment with a medicament that is a corticosteroid. Non-limiting examples of a corticosteroid include dexamethasone and prednisone. In certain embodiments, at least one prior line of therapy comprises treatment with a medicament that is an alkylating agent. In certain embodiments, at least one prior line of therapy comprises treatment with a medicament that is an anthracycline. In certain embodiments at least one prior line of therapy comprises treatment with a medicament that is an anti-CD38 antibody. Non-limiting examples of an anti-CD38 antibody include daratumumab, isatuximab and the investigational antibody TAK-079. In certain embodiments, at least one prior line of therapy comprises treatment with a medicament that is elotuzumab. In certain embodiments, at least one prior line of therapy comprises treatment with a medicament that is panobinostat. In certain embodiments, the subject has relapsed after said at least one prior line of therapy. In certain embodiments, the cancer is refractory to one or more of, or all of, bortezomib, carfilzomib, ixazomib, lenalidomide, pomalidomide, thalidomide, dexamethasone, prednisone, alkylating agents, daratumumab, isatuximab, TAK-079, elotuzumab and panobinostat. In certain embodiments prior lines of therapy include surgery, radiotherapy, or autologous or allogeneic transplant, or any combination of such treatments.

In some embodiments, the multiple myeloma is refractory to at least two medicaments. In some embodiments, the multiple myeloma is refractory to at least three medicaments. In some embodiments, the multiple myeloma is refractory to at least four medicaments. In some embodiments, the multiple myeloma is refractory to at least five medicaments.

In some embodiments, the subject has bone marrow plasma cells of between approximately 10% and approximately 30% before said administration of said CAR-T cells.

In certain embodiments, bone marrow aspirate or biopsy may be performed for clinical assessments or bone marrow aspirate may be performed for biomarker evaluations. In certain embodiments, clinical staging (morphology, cytogenetics, and immunohistochemistry or immunofluorescence or flow cytometry) may be done. In certain embodiments, a portion of the bone marrow aspirate may be immunophenotyped and monitored for BCMA, checkpoint ligand expression in CD138-positive multiple myeloma cells, and checkpoint expression on T cells. In certain embodiments, minimal residual disease (MRD) may be monitored in subjects using next generation sequencing (NGS) of bone marrow aspirate DNA. The NGS of bone marrow aspirate DNA is known to one of ordinary skill in the art. In certain embodiments, the NGS is performed via clonoSEQ. In certain embodiments, baseline bone marrow aspirates may be used to define the myeloma clones, and post-treatment samples may be used to evaluate MRD negativity. In certain embodiments, the MRD negativity status may be based on samples that are evaluable. In certain embodiments, evaluable samples are those that passed one or more of, or all of, calibration, quality control, and sufficiency of cells evaluable at a particular sensitivity level. In some embodiments, the sensitivity level is 10⁻⁶. In certain embodiments, the sensitivity level is 10⁻⁶, the sensitivity level is 10⁻⁵. In certain embodiments, the sensitivity level is 10⁻⁴. In certain embodiments, the sensitivity level is 10⁻³.

In certain embodiments, a subject’s response to the method of treatment is assessed using the International Myeloma Working Group (IMWG)-based response criteria, which are summarized in Table 2. In certain embodiments, the response may be classified as a stringent complete response (sCR). In certain embodiments, the response may be classified as a complete response (CR), which is worse than a stringent complete response (sCR). In certain embodiments, the response may be classified as a very good partial response (VGPR), which is worse than a complete response (CR). In certain embodiments, the response may be classified as a partial response (PR), which is worse than a very good partial response (VGPR). In certain embodiments, the response may be classified as a minimal response (MR), which is worse than a partial response (PR). In certain embodiments, the response may be classified as a stable disease (SD), which is worse than a minimal response (MR). In certain embodiments, the response may be classified as a progressive disease (PD), which is worse than a stable disease.

In certain embodiments, the tests used to assess International Myeloma Working Group (IMWG)-based response criteria are Myeloma protein (M-protein) measurements in serum and urine, serum calcium corrected for albumin, bone marrow examination, skeletal survey and documentation of extramedullary plasmacytomas.

Non-limiting examples of tests for M-protein measurement in blood and urine are known to one of ordinary skill in the art and comprise serum quantitative Ig, serum protein electrophoresis (SPEP), serum immunofixation electrophoresis, serum FLC assay, 24-hour urine M-protein quantitation by electrophoresis (UPEP), urine immunofixation electrophoresis, and serum β2-microglobulin.

Calculating serum calcium corrected for albumin in blood samples for detection of hypercalcemia is known to one of ordinary skill in the art. Without wishing to be bound by theory, calcium binds to albumin and only the unbound (free) calcium is biologically active; therefore, the serum calcium level must be adjusted for abnormal albumin levels (“corrected serum calcium”).

In certain embodiments, a skeletal survey of any one of, or all of, the skull, the entire vertebral column, the pelvis, the chest, the humeri, the femora, and any other bones, may be performed and evaluated by either roentgenography (“X-rays”) or low-dose computed tomography (CT) diagnostic quality scans without the use of IV contras, both of which are known to one of ordinary skill in the art. In certain embodiments, following T cell administration and before disease progression is confirmed, X-rays or CT scans may be performed locally, whenever clinically indicated based on symptoms, to document response or progression. In certain embodiments, magnetic resonance imaging (MRI) may be used for evaluating bone disease but does not replace a skeletal survey. MRI is known to one of ordinary skill in the art. In certain embodiments, if a radionuclide bone scan is used at screening, in addition to the complete skeletal survey, both methods may be used to document disease status. Radionuclide bone scans are known to one of ordinary skill in the art. In certain embodiments, the radionuclide bone scan and complete skeletal survey may be performed at the same time. In certain embodiments, a radionuclide bone scan may not replace a complete skeletal survey. In certain embodiments, if a subject presents with disease progression manifested by symptoms of pain due to bone changes, then disease progression may be documented by skeletal survey or other radiographs, depending on the symptoms that the subject experiences.

In certain embodiments, extramedullary plasmacytomas may be documented by clinical examination or MRI. In certain embodiments, if there was no contraindication to the use of IV contrast, extramedullary plasmacytomas may be documented by CT scan. In certain embodiments, extramedullary plasmacytomas may be documented by a fusion of positron emission tomography (PET) and CT scans if the CT component is of sufficient diagnostic quality. In certain embodiments, assessment of measurable sites of extramedullary disease may be performed, measured, or evaluated locally every 4 weeks for subjects until development of confirmed CR or confirmed disease progression. In certain embodiments, evaluation of extramedullary plasmacytomas may be done every 12 weeks.

In certain embodiments, to qualify for VGPR or PR or MR, the sum of products of the perpendicular diameters of the existing extramedullary plasmacytomas may have decreased by over 90% or at least 50%, respectively. In certain embodiments, to qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. In certain embodiments, to qualify for disease progression when not all existing extramedullary plasmacytomas are reported, the sum of products of the perpendicular diameters of the reported plasmacytomas had increased by at least 50%. In certain embodiments, if the study treatment interferes with the immunofixation assay, CR may be defined as the disappearance of the original M-protein associated with multiple myeloma on immunofixation.

In certain embodiments, a subject’s response to the method of treatment is assessed in terms of change in disease burden or tumor burden. Disease burden or tumor burden represents the type of measurable disease in the subject. In some embodiments, the change in tumor burden may be assessed in terms of paraprotein level changes upon treatment. In some embodiments, the paraprotein is an M-protein in the serum. In some embodiments, the paraprotein is an M-protein in the serum. In some embodiments, the change in tumor burden is assessed in terms of the difference between involved and uninvolved free light chain (dFLC). In some embodiments, the change in tumor burden is assessed in terms of the maximum paraprotein reduction from baseline, i.e., from prior to the administration of the CAR-T cells. In some embodiments, the change in tumor burden is assessed at a median follow-up time of greater than or equal to 28 days following the administration of CAR-T cells. In some embodiments, the change in tumor burden is assessed at a median follow-up time of greater than or equal to 1 month following the administration of CAR-T cells. In some embodiments, the change in tumor burden is assessed at a median follow-up time of greater than or equal to 3 months following the administration of CAR-T cells. In some embodiments, the change in tumor burden is assessed at a median follow-up time of greater than or equal to 6 months following the administration of CAR-T cells. In some embodiments, the change in tumor burden is assessed at a median follow-up time of greater than or equal to 9 months following the administration of CAR-T cells. In some embodiments, the change in tumor burden is assessed at a median follow-up time of greater than or equal to 12 months following the administration of CAR-T cells.

In certain embodiments, the subject is re-treated by administration via a second intravenous infusion of a second dose of CAR-T cells. In certain embodiments, the re-treatment dose comprises 1.0 × 10⁵ to 5.0 × 10⁶ of CAR-T cells per kilogram of the mass of the subject. In certain embodiments, the re-treatment dose comprises approximately 0.75 × 10⁵ of CAR-T cells per kilogram of the mass of the subject. In certain embodiments, the subject is re-treated upon exhibiting progressive disease after a best response of minimal response or better following the first infusion of CAR-T cells. In certain embodiments, the time between the first infusion of CAR-T cells and the detection of the progressive disease comprises at least six months.

### 4.8. Methods of Treating Lenalidomide-Refractory Subjects

In one aspect is provided a method of treating a subject, said method comprising administering to the subject a composition comprising a therapeutically effective number of T cells comprising a chimeric antigen receptor (CAR), wherein said subject has multiple myeloma and is lenalidomide-refractory. In some embodiments, the subject has received prior treatment with one, two or three prior lines of therapy.

In some embodiments, the multiple myeloma is refractory to the last line of therapy. In some embodiments, the subject has relapsed after said one, two or three prior lines of therapy. In some embodiments, the subject received prior treatment with at least one prior line of therapy comprising treatment with lenalidomide and at least one non-lenalidomide medicament, said at least one non-lenalidomide medicament comprising at least one of a proteasomal inhibitor, an immunomodulatory drug or an anti-CD38 antibody. In some embodiments, the subject has had no prior exposure to a BCMA-targeting medicament. In some embodiments, the subject received prior treatment with at least two prior lines of therapy. In some embodiments, the subject received prior treatment with three prior lines of therapy.

In some embodiments, the subject received prior treatment with dexamethasone, an alkylating agent or daratumumab. In some embodiments, the multiple myeloma is refractory to three classes of medicaments.

In certain embodiments, the method of treatment is effective in obtaining in the subject a reduction in tumor burden. In certain embodiments, the method of treatment is effective in obtaining in the subject a reduction in tumor burden of between approximately between approximately 1% and approximately 100%, between approximately 60% and approximately 100%, between approximately 65% and approximately 100%, between approximately 70% and approximately 100%, between approximately 75% and approximately 100%, between approximately 80% and approximately 100%, between approximately 85% and approximately 100%, between approximately 90% and approximately 100%, between approximately 92% and approximately 100%, between approximately 95% and approximately 100%, between approximately 96% and approximately 100%, between approximately 97% and approximately 100%, between approximately 98% and approximately 100%, or between approximately 99% and approximately 100%. In certain embodiments, the method of treatment is effective in obtaining in the subject a reduction in tumor burden of approximately 100%. In certain embodiments, the method of treatment is effective in obtaining in the subject a reduction in tumor burden of between approximately 1% and approximately 100% at a rate of between approximately 1% and approximately 100%. In certain embodiments, the method of treatment is effective in obtaining in the subject a reduction in tumor burden of between approximately 60% and approximately 100% at a rate of between approximately 1% and approximately 100%. In certain embodiments, the method of treatment is effective in obtaining in the subject a reduction in tumor burden of between approximately 65% and approximately 100% at a rate of between approximately 1% and approximately 92%. In certain embodiments, the method of treatment is effective in obtaining in the subject a reduction in tumor burden of between approximately 70% and approximately 100% at a rate of between approximately 1% and approximately 88%. In certain embodiments, the method of treatment is effective in obtaining in the subject a reduction in tumor burden of between approximately 90% and approximately 100% at a rate of between approximately 1% and approximately 88%. In certain embodiments, the method of treatment is effective in obtaining in the subject a reduction in tumor burden of between approximately 95% and approximately 100% at a rate of between approximately 1% and approximately 88%. In certain embodiments, the method of treatment is effective in obtaining in the subject a reduction in tumor burden of between approximately 99% and approximately 100% at a rate of between approximately 1% and approximately 88%. In certain embodiments, the method of treatment is effective in obtaining in the subject a reduction in tumor burden of approximately 100% at a rate of between approximately 1% and approximately 83%.

In certain embodiments, the method of treatment is effective in obtaining in the subject minimal residual disease (MRD) negative status or maintaining said minimal residual disease (MRD) status.

In certain embodiments, the method of treatment is effective in obtaining in the subject minimal residual disease (MRD) negative status. In certain embodiments, the method of treatment is effective in obtaining in the subject a minimal residual disease (MRD) negative status at a sensitivity level of 10⁻⁶. In certain embodiments, the method of treatment is effective in obtaining in the subject minimal residual disease (MRD) negative status at a sensitivity level of 10⁻⁵. In certain embodiments, the method of treatment is effective in obtaining in the subject minimal residual disease (MRD) negative status at a sensitivity level of 10⁻⁴. In certain embodiments, the method of treatment is effective in obtaining in the subject minimal residual disease (MRD) negative status at a sensitivity level of 10⁻³. In certain embodiments, the method of treatment is effective in obtaining MRD negative status when assessed in the bone marrow. In certain embodiments, the method of treatment is effective in maintaining the MRD negative status when assessed using a bone marrow sample that is evaluable. In certain embodiments, the method of treatment is effective in obtaining MRD negative status when assessed using bone marrow DNA. In some embodiments, said method is effective in obtaining minimal residual disease (MRD) negative status in said subject assessed in the bone marrow at a follow-up time of approximately 29 days or later after said administration of said CAR-T cells, approximately 2 months or later after said administration of said CAR-T cells, approximately 3 months or later after said administration of said CAR-T cells, approximately 6 months or later after said administration of said CAR-T cells, approximately 9 months or later after said administration of said CAR-T cells, or approximately 12 months or later after said administration of said CAR-T cells. In some embodiments, said minimal residual disease (MRD) negative status is obtained at a first follow-up time of between approximately 29 days and approximately 184 days after said administration of said CAR-T cells.

In certain embodiments, the method of treatment is effective in maintaining in the subject a first obtained minimal residual disease (MRD) negative status. In certain embodiments, the method of treatment is effective in maintaining MRD negative status at a sensitivity level of 10⁻⁵. In certain embodiments, the method of treatment is effective in obtaining in the subject minimal residual disease (MRD) negative status at a sensitivity level of 10⁻⁶. In certain embodiments, the method of treatment is effective in maintaining MRD negative status at a sensitivity level of 10⁻⁴. In certain embodiments, the method of treatment is effective in maintaining MRD negative status at a sensitivity level of 10⁻³. In certain embodiments, the method of treatment is effective in maintaining the MRD negative status when assessed using a bone marrow sample. In certain embodiments, the method of treatment is effective in maintaining the MRD negative status when assessed using a bone marrow sample that is evaluable. In certain embodiments, the method of treatment is effective in maintaining MRD negative status is maintained when assessed using bone marrow DNA. In some embodiments, said method is effective in maintaining said minimal residual disease (MRD) negative status in said subject assessed in the bone marrow at a follow-up time of between approximately 29 days and approximately 291 days after said administration of said CAR-T cells, between approximately 29 days and approximately 9 months after said administration of said CAR-T cells, between approximately 29 days and approximately 6 months after said administration of said CAR-T cells, between approximately 29 days and approximately 3 months after said administration of said CAR-T cells, or between approximately 29 days and approximately 2 months after said administration of said CAR-T cells. In some embodiments, said method is effective in maintaining said minimal residual disease (MRD) negative status in said subject assessed in the bone marrow at a second follow-up time of between approximately 57 days and approximately 191 days after said administration of said CAR-T cells, further wherein said first follow-up time is earlier than said second follow-up time.

In certain embodiments, the efficacy of the method of treatment is assessed by evaluating the proportion of subjects with MRD negative status. In certain embodiments, the efficacy of the method of treatment is assessed by evaluating the proportion of subjects with MRD negative status at a sensitivity level of 10⁻⁶. In certain embodiments, the efficacy of the method of treatment is assessed by evaluating the proportion of subjects with MRD negative status at a sensitivity level of 10⁻⁵. In certain embodiments, the efficacy of the method of treatment is assessed by evaluating the proportion of subjects with MRD negative status at a sensitivity level of 10⁻⁴. In certain embodiments, the efficacy of the method of treatment is assessed by evaluating the proportion of subjects with MRD negative status at a sensitivity level of 10⁻³. In certain embodiments, the efficacy of the method of treatment is assessed by evaluating the proportion of subjects with MRD negative status at a median follow-up time between the administration of the CAR-T cells and approximately 291 days after the administration of the CAR-T cells, between the administration of the CAR-T cells and approximately 9 months after the administration of the CAR-T cells, between the administration of the CAR-T cells and approximately 6 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 29 days after the administration of the CAR-T cells. In some embodiments, said method is effective in obtaining said minimal residual disease (MRD) negative status at a rate of approximately 24% or less at a sensitivity threshold level of 10⁻⁴, 10⁻⁵ or 10⁻⁶. In some embodiments, said method is effective in obtaining said minimal residual disease (MRD) negative status at a rate of approximately 41% or less at a sensitivity threshold level of 10⁻⁴, 10⁻⁵ or 10⁻⁶. In some embodiments, said method is effective in obtaining said minimal residual disease (MRD) negative status at a rate of approximately 61% or less at a sensitivity threshold level of 10⁻⁴, 10⁻⁵ or 10⁻⁶. In some embodiments, said method is effective in obtaining said minimal residual disease (MRD) negative status at a rate of between approximately 24% and approximately 61% at a sensitivity threshold level of 10⁻⁴, 10⁻⁵ or 10⁻⁶. In some embodiments, said method is effective in obtaining said minimal residual disease (MRD) negative status at a rate of approximately 41% at a sensitivity threshold level of 10⁻⁴, 10⁻⁵ or 10⁻⁶.

In certain embodiments, the efficacy of the method of treatment is assessed by evaluating the proportion of subjects with evaluable bone marrow and MRD negative status. In certain embodiments, the efficacy of the method of treatment is assessed by evaluating the proportion of subjects with evaluable bone marrow and MRD negative status at a sensitivity level of 10⁻⁶. In certain embodiments, the efficacy of the method of treatment is assessed by evaluating the proportion of subjects with evaluable bone marrow and MRD negative status at a sensitivity level of 10⁻⁵. In certain embodiments, the efficacy of the method of treatment is assessed by evaluating the proportion of subjects with evaluable bone marrow and MRD negative status at a sensitivity level of 10⁻⁴. In certain embodiments, the efficacy of the method of treatment is assessed by evaluating the proportion of subjects with evaluable bone marrow and MRD negative status at a sensitivity level of 10⁻³. In certain embodiments, the efficacy of the method of treatment is assessed by evaluating the proportion of subjects with evaluable bone marrow and MRD negative status at a median follow-up time between the administration of the CAR-T cells and approximately 291 days after the administration of the CAR-T cells, between the administration of the CAR-T cells and approximately 9 months after the administration of the CAR-T cells, between the administration of the CAR-T cells and approximately 6 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 29 days after the administration of the CAR-T cells. In some embodiments, said method is effective in obtaining said minimal residual disease (MRD) negative status at a rate of approximately 64% or less in subjects with evaluable samples at a sensitivity threshold level of 10⁻⁵. In some embodiments, said method is effective in obtaining said minimal residual disease (MRD) negative status at a rate of approximately 92% or less in subjects with evaluable samples at a sensitivity threshold level of 10⁻⁵. In some embodiments, said method is effective in obtaining said minimal residual disease (MRD) negative status at a rate of approximately 99% or less in subjects with evaluable samples at a sensitivity threshold level of 10⁻⁵. In some embodiments, said method is effective in obtaining said minimal residual disease (MRD) negative status at a rate of between approximately 64% and approximately 99% in subjects with evaluable samples at a sensitivity threshold level of 10⁻⁵. In some embodiments, said method is effective in obtaining said minimal residual disease (MRD) negative status at a rate of approximately 92% in subjects with evaluable samples at a sensitivity threshold level of 10⁻⁵.

In some embodiments, said method is effective in obtaining at least one response in the subject after said administration of said CAR-T cells, wherein said at least one response comprises, in order from better to worse, a stringent complete response, a complete response, a very good partial response, a partial response or a minimal response.

In some embodiments, said method is effective in obtaining a first response within approximately 21 days or later after said administration of said CAR-T cells. In some embodiments, said method is effective in obtaining a first response within approximately 30 days or later after said administration of said CAR-T cells. In some embodiments, said method is effective in obtaining a first response within approximately 36 days or later after said administration of said CAR-T cells. In some embodiments, said method is effective in obtaining a first response within approximately 56 days or later after said administration of said CAR-T cells. In some embodiments, said method is effective in obtaining a first response within approximately 99 days or later after said administration of said CAR-T cells. In some embodiments, said method is effective in obtaining a first response before a time of between approximately 21 days and approximately 99 days after said administration of said CAR-T cells. In some embodiments, said method is effective in obtaining a first response before a time of between approximately 21 days and approximately 55 days after said administration of said CAR-T cells. In some embodiments, said method is effective in obtaining a first response before approximately 36 days after said administration of said CAR-T cells. In some embodiments, said method is effective in obtaining a first response before approximately 30 days after said administration of said CAR-T cells.

In certain embodiments, the efficacy of the method of treatment is assessed by evaluating the proportion of subjects with a stringent complete response. In certain embodiments, the efficacy of the method of treatment is assessed by evaluating the proportion of subjects with a complete response or better. In certain embodiments, the efficacy of the method of treatment is assessed by evaluating the proportion of subjects with a very good partial response or better. In certain embodiments, the efficacy of the method of treatment is assessed by evaluating the proportion of subjects with a partial response or better. In certain embodiments, the efficacy of the method of treatment is assessed by evaluating the proportion of subjects with a minimal response or better.

In some embodiments, said method is effective in obtaining a best response of any one of minimal response, partial response, very good partial response, complete response or stringent complete response, i.e., a best response of minimal response or better. In some embodiments, the rate at which said method is effective in obtaining a best response of minimal response or better is called the clinical benefit rate. In some embodiments, said method is effective in obtaining said best response of minimal response or better at a rate of approximately 52% or less. In some embodiments, said method is effective in obtaining said best response of minimal response or better at a rate of approximately 72% or less. In some embodiments, said method is effective in obtaining said best response of minimal response or better at a rate of approximately 87% or less. In some embodiments, said method is effective in obtaining said best response of any one of minimal response, partial response, very good partial response, complete response or stringent complete response at a rate of between approximately 52% and approximately 87%. In some embodiments, said method is effective in obtaining said best response of any one of minimal response, partial response, very good partial response, complete response or stringent complete response at a rate of approximately 72%.

In some embodiments, said method is effective in obtaining a best response of any one of partial response, very good partial response, complete response or stringent complete response, i.e., a best response of partial response or better. In some embodiments, the rate at which said method is effective in obtaining a best response of partial response or better is called the overall survival rate or the overall response rate. In some embodiments, said method is effective in obtaining said best response of partial response or better at a rate of approximately 49% or less. In some embodiments, said method is effective in obtaining said best response of partial response or better at a rate of approximately 69% or less. In some embodiments, said method is effective in obtaining said best response of partial response or better at a rate of approximately 84% or less. In some embodiments, said method is effective in obtaining said best response of any one of partial response, very good partial response, complete response or stringent complete response at a rate of between approximately 49% and approximately 84%. In some embodiments, said method is effective in obtaining said best response of any one of partial response, very good partial response, complete response or stringent complete response at a rate of approximately 69%.

In some embodiments, said method is effective in obtaining a best response of any one of very good partial response, complete response or stringent complete response, i.e., a best response of very good partial response or better. In some embodiments, said method is effective in obtaining said best response of very good partial response or better at a rate of approximately 49% or less. In some embodiments, said method is effective in obtaining said best response of very good partial response or better at a rate of approximately 69% or less. In some embodiments, said method is effective in obtaining said best response of very good partial response or better at a rate of approximately 84% or less. In some embodiments, said method is effective in obtaining said best response of any one of very good partial response, complete response or stringent complete response at a rate of between approximately 49% and approximately 84%. In some embodiments, said method is effective in obtaining said best response of any one of very good partial response, complete response or stringent complete response at a rate of approximately 69%.

In some embodiments, said method is effective in obtaining a best response of complete response or stringent complete response, i.e., a best response of complete response or better. In some embodiments, said method is effective in obtaining said best response of complete response or better at a rate of approximately 39% or less. In some embodiments, said method is effective in obtaining said best response of complete response or better at a rate of approximately 58% or less. In some embodiments, said method is effective in obtaining said best response of complete response or better at a rate of approximately 76% or less. In some embodiments, said method is effective in obtaining said best response of complete response or stringent complete response at a rate of between approximately 39% and approximately 76%. In some embodiments, said method is effective in obtaining said best response of complete response or stringent complete response at a rate of approximately 58%.

In some embodiments, said method is effective in obtaining a best response of stringent complete response. In some embodiments, said method is effective in obtaining said best response of stringent complete response at a rate of approximately 33% or less. In some embodiments, said method is effective in obtaining said best response of stringent complete response at a rate of approximately 52% or less. In some embodiments, said method is effective in obtaining said best response of stringent complete response at a rate of approximately 70% or less. In some embodiments, said method is effective in obtaining said best response of stringent complete response at a rate of between approximately 33% and approximately 70%. In some embodiments, said method is effective in obtaining said best response of stringent complete response at a rate of approximately 52%.

In some embodiments, said method is effective in obtaining said best response within approximately 27 days or later after said administration of said CAR-T cells. In some embodiments, said method is effective in obtaining said best response within approximately 87 days or later after said administration of said CAR-T cells. In some embodiments, said method is effective in obtaining said best response within approximately 109 days or later after said administration of said CAR-T cells. In some embodiments, said method is effective in obtaining said best response within approximately 172 days or later after said administration of said CAR-T cells. In some embodiments, said method is effective in obtaining said best response within approximately 237 days or later after said administration of said CAR-T cells. In some embodiments, said method is effective in obtaining said best response before a time of between approximately 27 days and approximately 237 days after said administration of said CAR-T cells. In some embodiments, said method is effective in obtaining said best response before a time of between approximately 46 days and approximately 172 days after said administration of said CAR-T cells. In some embodiments, said method is effective in obtaining said best response before approximately 109 days after said administration of said CAR-T cells. In some embodiments, said method is effective in obtaining said best response before approximately 87 days after said administration of said CAR-T cells.

In some embodiments, said method is effective in maintaining a response in the subject at a follow-up time between the time of said first response and approximately 270 days after said administration of said CAR-T cells. In some embodiments, said method is effective in maintaining a response at a rate of between approximately 70% and approximately 99% at a follow-up time of approximately 6 months after said administration of said CAR-T cells. In some embodiments, said method is effective in maintaining a response at a rate of approximately 95% at a follow-up time of approximately 6 months after said administration of said CAR-T cells. In some embodiments, said method is effective in maintaining a response at a rate of between approximately 7% and approximately 92% at a follow-up time of approximately 9 months after said administration of said CAR-T cells. In some embodiments, said method is effective in maintaining a response at a rate of approximately 63% at a follow-up time of approximately 9 months after said administration of said CAR-T cells.

In some embodiments, wherein said method is effective in obtaining said first response within approximately 269 days or earlier after said administration of said CAR-T cells, said method is effective in maintaining a response in the subject at a follow-up time between the time of said first response and approximately 270 days after said administration of said CAR-T cells. In some embodiments, said method is effective in maintaining a response at a rate of approximately 70% or less at a follow-up time of 6 months after said administration of said CAR-T cells. In some embodiments, said method is effective in maintaining a response at a rate of approximately 95% or less at a follow-up time of 6 months after said administration of said CAR-T cells. In some embodiments, said method is effective in maintaining a response at a rate of approximately 99% or less at a follow-up time of 6 months after said administration of said CAR-T cells. In some embodiments, said method is effective in maintaining a response at a rate of approximately 63% or less at a follow-up time of 9 months after said administration of said CAR-T cells. In some embodiments, said method is effective in maintaining a response at a rate of approximately 92% or less at a follow-up time of 9 months after said administration of said CAR-T cells.

In some embodiments, said method is further effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells. In some embodiments, said method is effective in obtaining either minimal residual disease (MRD) negative complete response or minimal residual disease (MRD) negative stringent complete response at a rate of approximately 18% or less at a follow-up time of approximately 291 days after said administration of said CAR-T cells. In some embodiments, said method is effective in obtaining either minimal residual disease (MRD) negative complete response or minimal residual disease (MRD) negative stringent complete response at a rate of approximately 35% or less at a follow-up time of approximately 291 days after said administration of said CAR-T cells. In some embodiments, said method is effective in obtaining either minimal residual disease (MRD) negative complete response or minimal residual disease (MRD) negative stringent complete response at a rate of approximately 54% or less at a follow-up time of approximately 291 days after said administration of said CAR-T cells. In some embodiments, said method is effective in obtaining either minimal residual disease (MRD) negative complete response or minimal residual disease (MRD) negative stringent complete response at a rate of between approximately 18% and approximately 54% at a follow-up time of approximately 291 days after said administration of said CAR-T cells. In some embodiments, said method is effective in obtaining either minimal residual disease (MRD) negative complete response or minimal residual disease (MRD) negative stringent complete response at a rate of approximately 35% at a follow-up time of approximately 291 days after said administration of said CAR-T cells.

In some embodiments, said method is effective in obtaining progression-free survival of the subject. In some embodiments, said method is effective in obtaining said progression-free survival of the subject at a time between said administration of said CAR-T cells and approximately 55 days after said administration of said CAR-T cells. In some embodiments, said method is effective in obtaining said progression-free survival of the subject at a time between said administration of said CAR-T cells and approximately 297 days after said administration of said CAR-T cells. In some embodiments, said method is effective in obtaining said progression-free survival at a rate of approximately 62% or less at a follow-up time of approximately 6 months or approximately 9 months after said administration of said CAR-T cells. In some embodiments, said method is effective in obtaining said progression-free survival at a rate of approximately 86% or less at a follow-up time of approximately 6 months or approximately 9 months after said administration of said CAR-T cells. In some embodiments, said method is effective in obtaining said progression-free survival at a rate of approximately 95% or less at a follow-up time of approximately 6 months or approximately 9 months after said administration of said CAR-T cells. In some embodiments, said method is effective in obtaining said progression-free survival at a rate of between approximately 62% and approximately 95% at a follow-up time of approximately 6 months or approximately 9 months after said administration of said CAR-T cells. In some embodiments, said method is effective in obtaining said progression-free survival at a rate of approximately 86% at a follow-up time of approximately 6 months or approximately 9 months after said administration of said CAR-T cells.

In some embodiments, said method further comprises treating said subject for cytokine release syndrome more than approximately 3 days after said administration of said CAR-T cells. In some embodiments, the rate of recovery from cytokine release syndrome is approximately 90% or less 7 days after first observance of said cytokine release syndrome. In some embodiments, said method is effective in obtaining a rate of recovery from said cytokine release syndrome of between approximately 1% and approximately 90% at a time of approximately 7 days after first observance of said cytokine release syndrome.

In some embodiments, the rate of immune-effector cell associated neurotoxicity is approximately 20% or greater. In some embodiments, said method is effective in obtaining a rate of immune-effector cell associated neurotoxicity of between approximately 20% and approximately 99%.

### 4.9. Methods of Treating Subjects With Prior Early Relapse

In one aspect is provided a method of treating a subject, said method comprising administering to the subject a composition comprising a therapeutically effective number of T cells comprising a chimeric antigen receptor (CAR), wherein said subject has multiple myeloma and has had a prior early relapse. The term “prior early relapse” means disease progression per International Myeloma Working Group (IMWG)-based response criteria either: (i) between the time of treatment with autologous stem cell transplantation (ASCT) and approximately 12 months after said treatment with autologous stem cell transplantation (ASCT), for participants who have had autologous stem cell transplantation (ASCT); or (ii) between the time of start of anti-myeloma therapy and approximately 12 months from the start of anti-myeloma therapy, for participants who have not had autologous stem cell transplantation (ASCT).

In some embodiments, the subject has received prior treatment with one prior line of therapy. In some embodiments, said one prior line of therapy comprising treatment with at least two medicaments. In some embodiments, said at least two medicaments comprise a proteasomal inhibitor and an immunomodulatory drug. In some embodiments, the subject was additionally treated with an anti-CD38 antibody. In some embodiments, the subject has had no prior exposure to a BCMA-targeting medicament. In some embodiments, the multiple myeloma is refractory to at least one medicament.

In certain embodiments, the method of treatment is effective in obtaining in the subject a reduction in tumor burden. In certain embodiments, the method of treatment is effective in obtaining in the subject a reduction in tumor burden of between approximately between approximately 1% and approximately 100%, between approximately 60% and approximately 100%, between approximately 65% and approximately 100%, between approximately 70% and approximately 100%, between approximately 75% and approximately 100%, between approximately 80% and approximately 100%, between approximately 85% and approximately 100%, between approximately 90% and approximately 100%, between approximately 92% and approximately 100%, between approximately 95% and approximately 100%, between approximately 96% and approximately 100%, between approximately 97% and approximately 100%, between approximately 98% and approximately 100%, or between approximately 99% and approximately 100%. In certain embodiments, the method of treatment is effective in obtaining in the subject a reduction in tumor burden of approximately 100%. In certain embodiments, the method of treatment is effective in obtaining in the subject a reduction in tumor burden of between approximately 1% and approximately 100% at a rate of between approximately 1% and approximately 100%. In certain embodiments, the method of treatment is effective in obtaining in the subject a reduction in tumor burden of between approximately 80% and approximately 100% at a rate of between approximately 1% and approximately 100%. In certain embodiments, the method of treatment is effective in obtaining in the subject a reduction in tumor burden of between approximately 85% and approximately 100% at a rate of between approximately 1% and approximately 100%. In certain embodiments, the method of treatment is effective in obtaining in the subject a reduction in tumor burden of between approximately 90% and approximately 100% at a rate of between approximately 1% and approximately 88%. In certain embodiments, the method of treatment is effective in obtaining in the subject a reduction in tumor burden of between approximately 95% and approximately 100% at a rate of between approximately 1% and approximately 88%. In certain embodiments, the method of treatment is effective in obtaining in the subject a reduction in tumor burden of between approximately 96% and approximately 100% at a rate of between approximately 1% and approximately 82%. In certain embodiments, the method of treatment is effective in obtaining in the subject a reduction in tumor burden of between approximately 99% and approximately 100% at a rate of between approximately 1% and approximately 82%. In certain embodiments, the method of treatment is effective in obtaining in the subject a reduction in tumor burden of approximately 100% at a rate of between approximately 1% and approximately 76%.

In certain embodiments, the method of treatment is effective in obtaining in the subject minimal residual disease (MRD) negative status or maintaining said minimal residual disease (MRD) status.

In certain embodiments, the method of treatment is effective in obtaining in the subject minimal residual disease (MRD) negative status. In certain embodiments, the method of treatment is effective in obtaining in the subject a minimal residual disease (MRD) negative status at a sensitivity level of 10⁻⁶. In certain embodiments, the method of treatment is effective in obtaining in the subject minimal residual disease (MRD) negative status at a sensitivity level of 10⁻⁵. In certain embodiments, the method of treatment is effective in obtaining in the subject minimal residual disease (MRD) negative status at a sensitivity level of 10⁻⁴. In certain embodiments, the method of treatment is effective in obtaining in the subject minimal residual disease (MRD) negative status at a sensitivity level of 10⁻³. In certain embodiments, the method of treatment is effective in obtaining MRD negative status when assessed in the bone marrow. In certain embodiments, the method of treatment is effective in maintaining the MRD negative status when assessed using a bone marrow sample that is evaluable. In certain embodiments, the method of treatment is effective in obtaining MRD negative status when assessed using bone marrow DNA. In some embodiments, said method is effective in obtaining minimal residual disease (MRD) negative status in said subject assessed in the bone marrow at a follow-up time of approximately 35 days or later after said administration of said CAR-T cells, approximately 2 months or later after said administration of said CAR-T cells, approximately 3 months or later after said administration of said CAR-T cells, approximately 6 months or later after said administration of said CAR-T cells, approximately 9 months or later after said administration of said CAR-T cells, or approximately 12 months or later after said administration of said CAR-T cells. In some embodiments, said minimal residual disease (MRD) negative status is assessed in the bone marrow at a first follow-up time at a first follow-up time of between approximately 35 days and approximately 58 days after said administration of said CAR-T cells.

In certain embodiments, the method of treatment is effective in maintaining in the subject a first obtained minimal residual disease (MRD) negative status. In certain embodiments, the method of treatment is effective in maintaining MRD negative status at a sensitivity level of 10⁻⁵. In certain embodiments, the method of treatment is effective in obtaining in the subject minimal residual disease (MRD) negative status at a sensitivity level of 10⁻⁶. In certain embodiments, the method of treatment is effective in maintaining MRD negative status at a sensitivity level of 10⁻⁴. In certain embodiments, the method of treatment is effective in maintaining MRD negative status at a sensitivity level of 10⁻³. In certain embodiments, the method of treatment is effective in maintaining the MRD negative status when assessed using a bone marrow sample. In certain embodiments, the method of treatment is effective in maintaining the MRD negative status when assessed using a bone marrow sample that is evaluable. In certain embodiments, the method of treatment is effective in maintaining MRD negative status is maintained when assessed using bone marrow DNA. In some embodiments, said method is effective in maintaining said minimal residual disease (MRD) negative status in said subject assessed in the bone marrow at a follow-up time of between approximately 35 days and approximately 359 days after said administration of said CAR-T cells, between approximately 35 days and approximately 9 months after said administration of said CAR-T cells, between approximately 35 days and approximately 6 months after said administration of said CAR-T cells, between approximately 35 days and approximately 3 months after said administration of said CAR-T cells, or between approximately 35 days and approximately 2 months after said administration of said CAR-T cells. In some embodiments, said method is effective in maintaining said minimal residual disease (MRD) negative status in said subject assessed in the bone marrow at a second follow-up time of between approximately 78 days and approximately 359 days after said administration of said CAR-T cells, further wherein said first follow-up time is earlier than said second follow-up time.

In certain embodiments, the efficacy of the method of treatment is assessed by evaluating the proportion of subjects with MRD negative status. In certain embodiments, the efficacy of the method of treatment is assessed by evaluating the proportion of subjects with MRD negative status at a sensitivity level of 10⁻⁶. In certain embodiments, the efficacy of the method of treatment is assessed by evaluating the proportion of subjects with MRD negative status at a sensitivity level of 10⁻⁵. In certain embodiments, the efficacy of the method of treatment is assessed by evaluating the proportion of subjects with MRD negative status at a sensitivity level of 10⁻⁴. In certain embodiments, the efficacy of the method of treatment is assessed by evaluating the proportion of subjects with MRD negative status at a sensitivity level of 10⁻³.

In certain embodiments, the efficacy of the method of treatment is assessed by evaluating the proportion of subjects with MRD negative status at a median follow-up time between the administration of the CAR-T cells and approximately 141 days after the administration of the CAR-T cells, between the administration of the CAR-T cells and approximately 4 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 35 days after the administration of the CAR-T cells. In some embodiments, said method is effective in obtaining said minimal residual disease (MRD) negative status at a rate of approximately 26% or less at a sensitivity threshold level of 10⁻⁴ or 10⁻⁵ or approximately 17% or less at a sensitivity threshold level of 10⁻⁶. In some embodiments, said method is effective in obtaining said minimal residual disease (MRD) negative status at a rate of approximately 50% or less at a sensitivity threshold level of 10⁻⁴ or 10⁻⁵ or approximately 39% or less at a sensitivity threshold level of 10⁻⁶. In some embodiments, said method is effective in obtaining said minimal residual disease (MRD) negative status at a rate of approximately 74% or less at a sensitivity threshold level of 10⁻⁴ or 10⁻⁵ or approximately 64% or less at a sensitivity threshold level of 10⁻⁶. In some embodiments, said method is effective in obtaining said minimal residual disease (MRD) negative status at a rate of between approximately 26% and approximately 74% at a sensitivity threshold level of 10⁻⁴ or 10⁻⁵ or at a rate of between approximately 17% and approximately 64% at a sensitivity threshold level of 10⁻⁶. In some embodiments, said method is effective in obtaining said minimal residual disease (MRD) negative status at a rate of approximately 50% at a sensitivity threshold level of 10⁻⁴ or 10⁻⁵ or at a rate of approximately 39% at a sensitivity threshold level of 10⁻⁶.

In certain embodiments, the efficacy of the method of treatment is assessed by evaluating the proportion of subjects with evaluable bone marrow and MRD negative status. In certain embodiments, the efficacy of the method of treatment is assessed by evaluating the proportion of subjects with evaluable bone marrow and MRD negative status at a sensitivity level of 10⁻⁶. In certain embodiments, the efficacy of the method of treatment is assessed by evaluating the proportion of subjects with evaluable bone marrow and MRD negative status at a sensitivity level of 10⁻⁵. In certain embodiments, the efficacy of the method of treatment is assessed by evaluating the proportion of subjects with evaluable bone marrow and MRD negative status at a sensitivity level of 10⁻⁴. In certain embodiments, the efficacy of the method of treatment is assessed by evaluating the proportion of subjects with evaluable bone marrow and MRD negative status at a sensitivity level of 10⁻³. In certain embodiments, the efficacy of the method of treatment is assessed by evaluating the proportion of subjects with evaluable bone marrow and MRD negative status at a median follow-up time between the administration of the CAR-T cells and approximately 141 days after the administration of the CAR-T cells, between the administration of the CAR-T cells and approximately 4 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 35 days after the administration of the CAR-T cells. In some embodiments, said method is effective in obtaining said minimal residual disease (MRD) negative status at a rate of approximately 66% or less in subjects with evaluable samples at a sensitivity threshold level of 10⁻⁵. In some embodiments, said method is effective in obtaining said minimal residual disease (MRD) negative status at a rate of approximately 100% or less in subjects with evaluable samples at a sensitivity threshold level of 10⁻⁵. In some embodiments, said method is effective in obtaining said minimal residual disease (MRD) negative status at a rate of between approximately 66% and approximately 100% in subjects with evaluable samples at a sensitivity threshold level of 10⁻⁵. In some embodiments, said method is effective in obtaining said minimal residual disease (MRD) negative status at a rate of approximately 100% in subjects with evaluable samples at a sensitivity threshold level of 10⁻⁵.

In some embodiments, said method is effective in obtaining at least one response in the subject after said administration of said CAR-T cells, wherein said at least one response comprises, in order from better to worse, a stringent complete response, a complete response, a very good partial response, a partial response or a minimal response.

In some embodiments, said method is effective in obtaining a first response within approximately 27 days or later after said administration of said CAR-T cells. In some embodiments, said method is effective in obtaining a first response within approximately 28 days or later after said administration of said CAR-T cells. In some embodiments, said method is effective in obtaining a first response within approximately 33 days or later after said administration of said CAR-T cells. In some embodiments, said method is effective in obtaining a first response within approximately 46 days or later after said administration of said CAR-T cells. In some embodiments, said method is effective in obtaining a first response within approximately 78 days or later after said administration of said CAR-T cells. In some embodiments, said method is effective in obtaining a first response before a time of between approximately 27 days and approximately 78 days after said administration of said CAR-T cells. In some embodiments, said method is effective in obtaining a first response before a time of between approximately 27 days and approximately 47 days after said administration of said CAR-T cells. In some embodiments, n said method is effective in obtaining a first response before approximately 33 days after said administration of said CAR-T cells. In some embodiments, said method is effective in obtaining a first response before approximately 28 days after said administration of said CAR-T cells.

In certain embodiments, the efficacy of the method of treatment is assessed by evaluating the proportion of subjects with a stringent complete response. In certain embodiments, the efficacy of the method of treatment is assessed by evaluating the proportion of subjects with a complete response or better. In certain embodiments, the efficacy of the method of treatment is assessed by evaluating the proportion of subjects with a very good partial response or better. In certain embodiments, the efficacy of the method of treatment is assessed by evaluating the proportion of subjects with a partial response or better. In certain embodiments, the efficacy of the method of treatment is assessed by evaluating the proportion of subjects with a minimal response or better.

In some embodiments, said method is effective in obtaining a best response of any one of minimal response, partial response, very good partial response, complete response or stringent complete response, i.e., a best response of minimal response or better. In some embodiments, the rate at which said method is effective in obtaining a best response of minimal response or better is called the clinical benefit rate. In some embodiments, said method is effective in obtaining said best response of minimal response or better at a rate of approximately 65% or less. In some embodiments, said method is effective in obtaining said best response of minimal response or better at a rate of approximately 89% or less. In some embodiments, said method is effective in obtaining said best response of minimal response or better at a rate of approximately 99% or less. In some embodiments, said method is effective in obtaining said best response of any one of minimal response, partial response, very good partial response, complete response or stringent complete response at a rate of between approximately 65% and approximately 99%. In some embodiments, said method is effective in obtaining said best response of any one of minimal response, partial response, very good partial response, complete response or stringent complete response at a rate of approximately 89%.

In some embodiments, said method is effective in obtaining a best response of any one of partial response, very good partial response, complete response or stringent complete response, i.e., a best response of partial response or better. In some embodiments, the rate at which said method is effective in obtaining a best response of partial response or better is called the overall survival rate or the overall response rate. In some embodiments, said method is effective in obtaining said best response of partial response or better at a rate of approximately 65% or less. In some embodiments, said method is effective in obtaining said best response of partial response or better at a rate of approximately 89% or less. In some embodiments, said method is effective in obtaining said best response of partial response or better at a rate of approximately 99% or less. In some embodiments, said method is effective in obtaining said best response of any one of partial response, very good partial response, complete response or stringent complete response at a rate of between approximately 65% and approximately 99%. In some embodiments, said method is effective in obtaining said best response of any one of partial response, very good partial response, complete response or stringent complete response at a rate of approximately 89%.

In some embodiments, said method is effective in obtaining a best response of any one of very good partial response, complete response or stringent complete response, i.e., a best response of very good partial response or better. In some embodiments, said method is effective in obtaining said best response of very good partial response or better at a rate of approximately 41% or less. In some embodiments, said method is effective in obtaining said best response of very good partial response or better at a rate of approximately 67% or less. In some embodiments, said method is effective in obtaining said best response of very good partial response or better at a rate of approximately 87% or less. In some embodiments, said method is effective in obtaining said best response of any one of very good partial response, complete response or stringent complete response at a rate of between approximately 41% and approximately 87%. In some embodiments, said method is effective in obtaining said best response of any one of very good partial response, complete response or stringent complete response at a rate of approximately 67%.

In some embodiments, said method is effective in obtaining a best response of complete response or stringent complete response, i.e., a best response of complete response or better. In some embodiments, said method is effective in obtaining said best response of complete response or better at a rate of approximately 10% or less. In some embodiments, said method is effective in obtaining said best response of complete response or better at a rate of approximately 28% or less. In some embodiments, said method is effective in obtaining said best response of complete response or better at a rate of approximately 54% or less. In some embodiments, said method is effective in obtaining said best response of complete response or stringent complete response at a rate of between and approximately 10% and approximately 54%. In some embodiments, said method is effective in obtaining said best response of complete response or stringent complete response at a rate of approximately 28%.

In some embodiments, said method is effective in obtaining a best response of stringent complete response. In some embodiments, said method is effective in obtaining said best response of stringent complete response at a rate of approximately 6% or less. In some embodiments, said method is effective in obtaining said best response of stringent complete response at a rate of approximately 22% or less. In some embodiments, said method is effective in obtaining said best response of stringent complete response at a rate of approximately 48% or less. In some embodiments, said method is effective in obtaining said best response of stringent complete response at a rate of between approximately 6% and approximately 48%. In some embodiments, said method is effective in obtaining said best response of stringent complete response at a rate of approximately 22%.

In some embodiments, said method is effective in obtaining said best response within approximately 27 days or later after said administration of said CAR-T cells. In some embodiments, said method is effective in obtaining said best response within approximately 42 days or later after said administration of said CAR-T cells. In some embodiments, said method is effective in obtaining said best response within approximately 71 days or later after said administration of said CAR-T cells. In some embodiments, said method is effective in obtaining said best response within approximately 154 days or later after said administration of said CAR-T cells. In some embodiments, said method is effective in obtaining said best response within approximately 354 days or later after said administration of said CAR-T cells. In some embodiments, said method is effective in obtaining said best response before a time of between approximately 27 days and approximately 354 days after said administration of said CAR-T cells. In some embodiments, said method is effective in obtaining said best response before a time of between approximately 27 days and approximately 155 days after said administration of said CAR-T cells. In some embodiments, said method is effective in obtaining said best response before approximately 71 days after said administration of said CAR-T cells. In some embodiments, said method is effective in obtaining said best response before approximately 42 days after said administration of said CAR-T cells.

In some embodiments, said method is effective in maintaining a response in the subject at a follow-up time of between the time of said first response and approximately 156 days after said administration of said CAR-T cells. In some embodiments, said method is effective in maintaining a response at a rate of between approximately 5% and approximately 95% at a follow-up time of approximately 6 months, approximately 9 months or approximately 12 months after said administration of said CAR-T cells. In some embodiments, said method is effective in maintaining a response at a rate of approximately 67% at a follow-up time of approximately 6 months, approximately 9 months or approximately 12 months after said administration of said CAR-T cells.

In some embodiments, wherein said method is effective in obtaining said first response within approximately 269 days or earlier after said administration of said CAR-T cells, said method is effective in maintaining a response in the subject at a follow-up time of between the time of said first response and approximately 156 days after said administration of said CAR-T cells. In some embodiments, said method is effective in maintaining a response at a rate of approximately 5% or less at a follow-up time of 6 months, 9 months or 12 months after said administration of said CAR-T cells. In some embodiments, said method is effective in maintaining a response at a rate of approximately 67% or less at a follow-up time of 6 months, 9 months or 12 months after said administration of said CAR-T cells. In some embodiments, said method is effective in maintaining a response at a rate of approximately 95% or less at a follow-up time of 6 months, 9 months or 12 months after said administration of said CAR-T cells.

In some embodiments, said method is further effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells. In some embodiments, said method is effective in obtaining either minimal residual disease (MRD) negative complete response or minimal residual disease (MRD) negative stringent complete response at a rate of approximately 1% or less at a follow-up time of approximately 141 days after said administration of said CAR-T cells. In some embodiments, said method is effective in obtaining either minimal residual disease (MRD) negative complete response or minimal residual disease (MRD) negative stringent complete response at a rate of approximately 11% or less at a follow-up time of approximately 141 days after said administration of said CAR-T cells. In some embodiments, said method is effective in obtaining either minimal residual disease (MRD) negative complete response or minimal residual disease (MRD) negative stringent complete response at a rate of approximately 35% or less at a follow-up time of approximately 141 days after said administration of said CAR-T cells. In some embodiments, said method is effective in obtaining either minimal residual disease (MRD) negative complete response or minimal residual disease (MRD) negative stringent complete response at a rate of between approximately 1% and approximately 35% at a follow-up time of approximately 141 days after said administration of said CAR-T cells. In some embodiments, said method is effective in obtaining either minimal residual disease (MRD) negative complete response or minimal residual disease (MRD) negative stringent complete response at a rate of approximately 11% at a follow-up time of approximately 141 days after said administration of said CAR-T cells.

In some embodiments, said method is effective in obtaining progression-free survival of the subject. In some embodiments, said method is effective in obtaining said progression-free survival of the subject at a time between said administration of said CAR-T cells and approximately 182 days after said administration of said CAR-T cells. In some embodiments, said method is effective in obtaining said progression-free survival at a rate of approximately 100% or less at a follow-up time of 6 months after said administration of said CAR-T cells. In some embodiments, said method is effective in obtaining said progression-free survival at a rate of approximately 5% or less at a follow-up time of approximately 9 months or approximately 12 months after said administration of said CAR-T cells. In some embodiments, said method is effective in obtaining said progression-free survival at a rate of approximately 67% or less at a follow-up time of approximately 9 months or approximately 12 months after said administration of said CAR-T cells. In some embodiments, said method is effective in obtaining said progression-free survival at a rate of approximately 95% or less at a follow-up time of approximately 9 months or approximately 12 months after said administration of said CAR-T cells. In some embodiments, said method is effective in obtaining said progression-free survival at a rate of approximately 100% at a follow-up time of approximately 6 months after said administration of said CAR-T cells. In some embodiments, said method is effective in obtaining said progression-free survival at a rate of between approximately 5% and approximately 95% at a follow-up time of approximately 9 months or approximately 12 months after said administration of said CAR-T cells. In some embodiments, said method is effective in obtaining said progression-free survival at a rate of approximately 67% at a follow-up time of approximately 9 months or approximately 12 months after said administration of said CAR-T cells.

In some embodiments, the rate of recovery from cytokine release syndrome is approximately 100% or less 7 days after first observance of said cytokine release syndrome. In some embodiments, said method is effective obtaining a rate of recovery from said cytokine release syndrome of between approximately 1% and approximately 100% at a time of approximately 7 days after first observance of said cytokine release syndrome.

### 4.10. Methods of Treating Subjects With Prior Non-Cellular BCMA-Targeted Treatment

In one aspect is provided a method of treating a subject, said method comprising administering to the subject a composition comprising a therapeutically effective number of T cells comprising a chimeric antigen receptor (CAR), wherein said subject has multiple myeloma and has received at least one prior line of therapy comprising treatment with a non-cellular BCMA-targeting medicament. In some embodiments, said at least one prior line of therapy comprises treatment with at least four medicaments, wherein said at least four medicaments comprises a non-cellular BCMA-targeting medicament. In some embodiments, said at least four medicaments further comprises a proteasomal inhibitor, an immunomodulatory drug and an anti-CD38 antibody.

In some embodiments, the subject received prior treatment with at least two prior lines of therapy, at least three prior lines of therapy, at least four prior lines of therapy, at least five prior lines of therapy, at least six prior lines of therapy, at least seven prior lines of therapy, at least eight prior lines of therapy, at least nine prior lines of therapy, at least ten prior lines of therapy, at least eleven prior lines of therapy, or at least twelve prior lines of therapy. In some embodiments, the subject has relapsed after said at least one prior line of therapy, at least two prior lines of therapy, at least three prior lines of therapy, at least four prior lines of therapy, at least five prior lines of therapy, at least six prior lines of therapy, at least seven prior lines of therapy, at least eight prior lines of therapy, at least nine prior lines of therapy, at least ten prior lines of therapy, at least eleven prior lines of therapy, or at least twelve prior lines of therapy.

In certain embodiments, the method of treatment is effective in obtaining in the subject a reduction in tumor burden. In certain embodiments, the method of treatment is effective in obtaining in the subject a reduction in tumor burden of between approximately between approximately 1% and approximately 100%, between approximately 20% and approximately 100%, between approximately 60% and approximately 100%, between approximately 65% and approximately 100%, between approximately 70% and approximately 100%, between approximately 75% and approximately 100%, between approximately 80% and approximately 100%, between approximately 85% and approximately 100%, between approximately 90% and approximately 100%, between approximately 92% and approximately 100%, between approximately 95% and approximately 100%, between approximately 96% and approximately 100%, between approximately 97% and approximately 100%, between approximately 98% and approximately 100%, or between approximately 99% and approximately 100%. In certain embodiments, the method of treatment is effective in obtaining in the subject a reduction in tumor burden of approximately 100%. In certain embodiments, the method of treatment is effective in obtaining in the subject a reduction in tumor burden of between approximately 1% and approximately 100% at a rate of between approximately 1% and approximately 83%. In certain embodiments, the method of treatment is effective in obtaining in the subject a reduction in tumor burden of between approximately 5% and approximately 100% at a rate of between approximately 1% and approximately 77%. In certain embodiments, the method of treatment is effective in obtaining in the subject a reduction in tumor burden of between approximately 20% and approximately 100% at a rate of between approximately 1% and approximately 77%. In certain embodiments, the method of treatment is effective in obtaining in the subject a reduction in tumor burden of between approximately 30% and approximately 100% at a rate of between approximately 1% and approximately 77%. In certain embodiments, the method of treatment is effective in obtaining in the subject a reduction in tumor burden of between approximately 40% and approximately 100% at a rate of between approximately 1% and approximately 77%. In certain embodiments, the method of treatment is effective in obtaining in the subject a reduction in tumor burden of between approximately 50% and approximately 100% at a rate of between approximately 1% and approximately 77%. In certain embodiments, the method of treatment is effective in obtaining in the subject a reduction in tumor burden of between approximately 60% and approximately 100% at a rate of between approximately 1% and approximately 72%. In certain embodiments, the method of treatment is effective in obtaining in the subject a reduction in tumor burden of between approximately 70% and approximately 100% at a rate of between approximately 1% and approximately 72%. In certain embodiments, the method of treatment is effective in obtaining in the subject a reduction in tumor burden of between approximately 80% and approximately 100% at a rate of between approximately 1% and approximately 67%. In certain embodiments, the method of treatment is effective in obtaining in the subject a reduction in tumor burden of between approximately 90% and approximately 100% at a rate of between approximately 1% and approximately 67%. In certain embodiments, the method of treatment is effective in obtaining in the subject a reduction in tumor burden of between approximately 95% and approximately 100% at a rate of between approximately 1% and approximately 61%. In certain embodiments, the method of treatment is effective in obtaining in the subject a reduction in tumor burden of approximately 100% at a rate of between approximately 1% and approximately 50%.

In certain embodiments, the method of treatment is effective in obtaining in the subject minimal residual disease (MRD) negative status or maintaining said minimal residual disease (MRD) status.

In certain embodiments, the method of treatment is effective in obtaining in the subject minimal residual disease (MRD) negative status. In certain embodiments, the method of treatment is effective in obtaining in the subject a minimal residual disease (MRD) negative status at a sensitivity level of 10⁻⁶. In certain embodiments, the method of treatment is effective in obtaining in the subject minimal residual disease (MRD) negative status at a sensitivity level of 10⁻⁵. In certain embodiments, the method of treatment is effective in obtaining in the subject minimal residual disease (MRD) negative status at a sensitivity level of 10⁻⁴. In certain embodiments, the method of treatment is effective in obtaining in the subject minimal residual disease (MRD) negative status at a sensitivity level of 10⁻³. In certain embodiments, the method of treatment is effective in obtaining MRD negative status when assessed in the bone marrow. In certain embodiments, the method of treatment is effective in maintaining the MRD negative status when assessed using a bone marrow sample that is evaluable. In certain embodiments, the method of treatment is effective in obtaining MRD negative status when assessed using bone marrow DNA. In some embodiments, said method is effective in obtaining minimal residual disease (MRD) negative status in said subject assessed in the bone marrow at a follow-up time of approximately 27 days or later after said administration of said CAR-T cells, approximately 2 months or later after said administration of said CAR-T cells, approximately 3 months or later after said administration of said CAR-T cells, approximately 6 months or later after said administration of said CAR-T cells, approximately 9 months or later after said administration of said CAR-T cells, or approximately 12 months or later after said administration of said CAR-T cells. In some embodiments, said minimal residual disease (MRD) negative status is assessed in the bone marrow at a first follow-up time at a first follow-up time of between approximately 56 days and approximately 58 days after said administration of said CAR-T cells.

In certain embodiments, the method of treatment is effective in maintaining in the subject a first obtained minimal residual disease (MRD) negative status. In certain embodiments, the method of treatment is effective in maintaining MRD negative status at a sensitivity level of 10⁻⁵. In certain embodiments, the method of treatment is effective in obtaining in the subject minimal residual disease (MRD) negative status at a sensitivity level of 10⁻⁶. In certain embodiments, the method of treatment is effective in maintaining MRD negative status at a sensitivity level of 10⁻⁴. In certain embodiments, the method of treatment is effective in maintaining MRD negative status at a sensitivity level of 10⁻³. In certain embodiments, the method of treatment is effective in maintaining the MRD negative status when assessed using a bone marrow sample. In certain embodiments, the method of treatment is effective in maintaining the MRD negative status when assessed using a bone marrow sample that is evaluable. In certain embodiments, the method of treatment is effective in maintaining MRD negative status is maintained when assessed using bone marrow DNA. In some embodiments, said method is effective in maintaining said minimal residual disease (MRD) negative status in said subject assessed in the bone marrow at a follow-up time of between approximately 27 days and approximately 186 days after said administration of said CAR-T cells, between approximately 29 days and approximately 6 months after said administration of said CAR-T cells, between approximately 29 days and approximately 3 months after said administration of said CAR-T cells, or between approximately 29 days and approximately 2 months after said administration of said CAR-T cells. In some embodiments, said method is effective in maintaining said minimal residual disease (MRD) negative status in said subject assessed in the bone marrow at a second follow-up time of between approximately 183 days and approximately 186 days after said administration of said CAR-T cells, further wherein said first follow-up time is earlier than said second follow-up time.

In certain embodiments, the efficacy of the method of treatment is assessed by evaluating the proportion of subjects with MRD negative status. In certain embodiments, the efficacy of the method of treatment is assessed by evaluating the proportion of subjects with MRD negative status at a sensitivity level of 10⁻⁶. In certain embodiments, the efficacy of the method of treatment is assessed by evaluating the proportion of subjects with MRD negative status at a sensitivity level of 10⁻⁵. In certain embodiments, the efficacy of the method of treatment is assessed by evaluating the proportion of subjects with MRD negative status at a sensitivity level of 10⁻⁴. In certain embodiments, the efficacy of the method of treatment is assessed by evaluating the proportion of subjects with MRD negative status at a sensitivity level of 10⁻³. In certain embodiments, the efficacy of the method of treatment is assessed by evaluating the proportion of subjects with MRD negative status at a median follow-up time between the administration of the CAR-T cells and approximately 186 days after the administration of the CAR-T cells, between the administration of the CAR-T cells and approximately 6 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 27 days after the administration of the CAR-T cells. In some embodiments, said method is effective in obtaining said minimal residual disease (MRD) negative status at a rate of approximately 9% or less at a sensitivity threshold level of 10⁻⁴, approximately 6% or less at a sensitivity threshold level of 10⁻⁵, or approximately 1% or less at a sensitivity threshold level of 10⁻⁶. In some embodiments, said method is effective in obtaining said minimal residual disease (MRD) negative status at a rate of approximately 25% or less at a sensitivity threshold level of 10⁻⁴, approximately 20% or less at a sensitivity threshold level of 10⁻⁵, or approximately 10% or less at a sensitivity threshold level of 10⁻⁶. In some embodiments, said method is effective in obtaining said minimal residual disease (MRD) negative status at a rate of approximately 49% or less at a sensitivity threshold level of 10⁻⁴, approximately 44% or less at a sensitivity threshold level of 10⁻⁵, or approximately 31% or less at a sensitivity threshold level of 10⁻⁶. In some embodiments, said method is effective in obtaining said minimal residual disease (MRD) negative status at a rate of between approximately 9% and approximately 49% at a sensitivity threshold level of 10⁻⁴, at a rate of between approximately 6% and approximately 44% at a sensitivity threshold level of 10⁻⁵, or at a rate of between approximately 1% and approximately 31% at a sensitivity threshold level of 10⁻⁶. In some embodiments, said method is effective in obtaining said minimal residual disease (MRD) negative status at a rate of approximately 25% at a sensitivity threshold level of 10⁻⁴, at a rate of approximately 20% at a sensitivity threshold level of 10⁻⁵, or at a rate of approximately 10% at a sensitivity threshold level of 10⁻⁶.

In certain embodiments, the efficacy of the method of treatment is assessed by evaluating the proportion of subjects with evaluable bone marrow and MRD negative status. In certain embodiments, the efficacy of the method of treatment is assessed by evaluating the proportion of subjects with evaluable bone marrow and MRD negative status at a sensitivity level of 10⁻⁶. In certain embodiments, the efficacy of the method of treatment is assessed by evaluating the proportion of subjects with evaluable bone marrow and MRD negative status at a sensitivity level of 10⁻⁵. In certain embodiments, the efficacy of the method of treatment is assessed by evaluating the proportion of subjects with evaluable bone marrow and MRD negative status at a sensitivity level of 10⁻⁴. In certain embodiments, the efficacy of the method of treatment is assessed by evaluating the proportion of subjects with evaluable bone marrow and MRD negative status at a sensitivity level of 10⁻³. In certain embodiments, the efficacy of the method of treatment is assessed by evaluating the proportion of subjects with evaluable bone marrow and MRD negative status at a median follow-up time between the administration of the CAR-T cells and approximately 186 days after the administration of the CAR-T cells, between the administration of the CAR-T cells and approximately 6 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 27 days after the administration of the CAR-T cells. In some embodiments, said method is effective in obtaining said minimal residual disease (MRD) negative status at a rate of approximately 22% or less in subjects with evaluable samples at a sensitivity threshold level of 10⁻⁵. In some embodiments, said method is effective in obtaining said minimal residual disease (MRD) negative status at a rate of approximately 67% or less in subjects with evaluable samples at a sensitivity threshold level of 10⁻⁵. In some embodiments, said method is effective in obtaining said minimal residual disease (MRD) negative status at a rate of approximately 96% or less in subjects with evaluable samples at a sensitivity threshold level of 10⁻⁵. In some embodiments, said method is effective in obtaining said minimal residual disease (MRD) negative status at a rate of between approximately 22% and approximately 96% in subjects with evaluable samples at a sensitivity threshold level of 10⁻⁵. In some embodiments, said method is effective in obtaining said minimal residual disease (MRD) negative status at a rate of approximately 67% in subjects with evaluable samples at a sensitivity threshold level of 10⁻⁵.

In some embodiments, said method is effective in obtaining at least one response in the subject after said administration of said CAR-T cells, wherein said at least one response comprises, in order from better to worse, a stringent complete response, a complete response, a very good partial response, a partial response or a minimal response.

In some embodiments, said method is effective in obtaining a first response within approximately 27 days or later after said administration of said CAR-T cells. In some embodiments, said method is effective in obtaining a first response within approximately 28 days or later after said administration of said CAR-T cells. In some embodiments, said method is effective in obtaining a first response within approximately 43 days or later after said administration of said CAR-T cells. In some embodiments, said method is effective in obtaining a first response within approximately 87 days or later after said administration of said CAR-T cells. In some embodiments, said method is effective in obtaining a first response within approximately 153 days or later after said administration of said CAR-T cells. In some embodiments, said method is effective in obtaining a first response before a time of between approximately 27 days and approximately 153 days after said administration of said CAR-T cells. In some embodiments, said method is effective in obtaining a first response before a time of between approximately 27 days and approximately 88 days after said administration of said CAR-T cells. In some embodiments, said method is effective in obtaining a first response before approximately 43 days after said administration of said CAR-T cells. In some embodiments, said method is effective in obtaining a first response before approximately 28 days after said administration of said CAR-T cells.

In certain embodiments, the efficacy of the method of treatment is assessed by evaluating the proportion of subjects with a stringent complete response. In certain embodiments, the efficacy of the method of treatment is assessed by evaluating the proportion of subjects with a complete response or better. In certain embodiments, the efficacy of the method of treatment is assessed by evaluating the proportion of subjects with a very good partial response or better. In certain embodiments, the efficacy of the method of treatment is assessed by evaluating the proportion of subjects with a partial response or better. In certain embodiments, the efficacy of the method of treatment is assessed by evaluating the proportion of subjects with a minimal response or better.

In some embodiments, said method is effective in obtaining a best response of any one of minimal response, partial response, very good partial response, complete response or stringent complete response, i.e., a best response of minimal response or better. In some embodiments, the rate at which said method is effective in obtaining a best response of minimal response or better is called the clinical benefit rate. In some embodiments, said method is effective in obtaining said best response of minimal response or better at a rate of approximately 23% or less. In some embodiments, said method is effective in obtaining said best response of minimal response or better at a rate of approximately 45% or less. In some embodiments, said method is effective in obtaining said best response of minimal response or better at a rate of approximately 68% or less. In some embodiments, said method is effective in obtaining said best response of any one of minimal response, partial response, very good partial response, complete response or stringent complete response at a rate of between approximately 23% and approximately 69%. In some embodiments, said method is effective in obtaining said best response of any one of minimal response, partial response, very good partial response, complete response or stringent complete response at a rate of approximately 45%.

In some embodiments, said method is effective in obtaining a best response of any one of partial response, very good partial response, complete response or stringent complete response, i.e., a best response of partial response or better. In some embodiments, the rate at which said method is effective in obtaining a best response of partial response or better is called the overall survival rate or the overall response rate. In some embodiments, said method is effective in obtaining said best response of partial response or better at a rate of approximately 19% or less. In some embodiments, said method is effective in obtaining said best response of partial response or better at a rate of approximately 40% or less. In some embodiments, said method is effective in obtaining said best response of partial response or better at a rate of approximately 63% or less. In some embodiments, aid method is effective in obtaining said best response of any one of partial response, very good partial response, complete response or stringent complete response at a rate of between approximately 19% and approximately 64%. In some embodiments, said method is effective in obtaining said best response of any one of partial response, very good partial response, complete response or stringent complete response at a rate of approximately 40%.

In some embodiments, said method is effective in obtaining a best response of any one of very good partial response, complete response or stringent complete response, i.e., a best response of very good partial response or better. In some embodiments, said method is effective in obtaining said best response of very good partial response or better at a rate of approximately 15% or less. In some embodiments, said method is effective in obtaining said best response of very good partial response or better at a rate of approximately 35% or less. In some embodiments, said method is effective in obtaining said best response of very good partial response or better at a rate of approximately 59% or less. In some embodiments, said method is effective in obtaining said best response of any one of very good partial response, complete response or stringent complete response at a rate of between approximately 15% and approximately 59%. In some embodiments, said method is effective in obtaining said best response of any one of very good partial response, complete response or stringent complete response at a rate of approximately 35%.

In some embodiments, said method is effective in obtaining a best response of complete response or stringent complete response, i.e., a best response of complete response or better. In some embodiments, said method is effective in obtaining said best response of complete response or better at a rate of approximately 3% or less. In some embodiments, said method is effective in obtaining said best response of complete response or better at a rate of approximately 15% or less. In some embodiments, said method is effective in obtaining said best response of complete response or better at a rate of approximately 38% or less. In some embodiments, said method is effective in obtaining said best response of complete response or stringent complete response at a rate of between approximately 3% and approximately 38%. In some embodiments, said method is effective in obtaining said best response of complete response or stringent complete response at a rate of approximately 15%.

In some embodiments, said method is effective in obtaining a best response of stringent complete response. In some embodiments, said method is effective in obtaining said best response of stringent complete response at a rate of approximately 1% or less. In some embodiments, said method is effective in obtaining said best response of stringent complete response at a rate of approximately 10% or less. In some embodiments, said method is effective in obtaining said best response of stringent complete response at a rate of approximately 32% or less. In some embodiments, said method is effective in obtaining said best response of stringent complete response at a rate of between approximately 1% and approximately 32%. In some embodiments, said method is effective in obtaining said best response of stringent complete response at a rate of approximately 10%.

In some embodiments, said method is effective in obtaining said best response within approximately 27 days or later after said administration of said CAR-T cells. In some embodiments, said method is effective in obtaining said best response within approximately 56 days or later after said administration of said CAR-T cells. In some embodiments, said method is effective in obtaining said best response within approximately 77 days or later after said administration of said CAR-T cells. In some embodiments, said method is effective in obtaining said best response within approximately 132 days or later after said administration of said CAR-T cells. In some embodiments, said method is effective in obtaining said best response within approximately 171 days or later after said administration of said CAR-T cells. In some embodiments, said method is effective in obtaining said best response before a time of between approximately 27 days and approximately 171 days after said administration of said CAR-T cells. In some embodiments, said method is effective in obtaining said best response before a time of between approximately 27 days and approximately 133 days after said administration of said CAR-T cells. In some embodiments, said method is effective in obtaining said best response before approximately 78 days after said administration of said CAR-T cells. In some embodiments, said method is effective in obtaining said best response before approximately 56 days after said administration of said CAR-T cells.

In some embodiments, said method is effective in maintaining a response in the subject at a follow-up time of between the time of said first response and approximately 132 days after said administration of said CAR-T cells, further wherein said first response was obtained between the time of said administration of said CAR-T cells and approximately 131 days after said administration of said CAR-T cells. In some embodiments, said method is effective in maintaining a response at a rate of between approximately 20% and approximately 96% at a follow-up time of approximately 6 months after said administration of said CAR-T cells. In some embodiments, said method is effective in maintaining a response at a rate of approximately 80% at a follow-up time of approximately 6 months after said administration of said CAR-T cells.

In some embodiments, wherein said method is effective in obtaining said first response within approximately 131 days or earlier after said administration of said CAR-T cells, said method is effective in maintaining a response in the subject at a follow-up time of between the time of said first response and approximately 132 days after said administration of said CAR-T cells. In some embodiments, said method is effective in maintaining a response at a rate of approximately 20% or less at a follow-up time of 6 months after said administration of said CAR-T cells. In some embodiments, said method is effective in maintaining a response at a rate of approximately 80% or less at a follow-up time of 6 months after said administration of said CAR-T cells. In some embodiments, said method is effective in maintaining a response at a rate of approximately 96% or less at a follow-up time of 6 months after said administration of said CAR-T cells.

In some embodiments, said method is effective in obtaining progression-free survival of the subject. In some embodiments, said method is effective in obtaining said progression-free survival of the subject at a time between said administration of said CAR-T cells and approximately 15 days after said administration of said CAR-T cells. In some embodiments, said method is effective in obtaining said progression-free survival of the subject at a time between said administration of said CAR-T cells and approximately 44 days after said administration of said CAR-T cells. In some embodiments, said method is effective in obtaining said progression-free survival of the subject at a time between said administration of said CAR-T cells and approximately 159 days after said administration of said CAR-T cells. In some embodiments, said method is effective in obtaining said progression-free survival at a rate of approximately 29% or less at a follow-up time of 6 months or 9 months after said administration of said CAR-T cells. In some embodiments, said method is effective in obtaining said progression-free survival at a rate of approximately 55% or less at a follow-up time of 6 months or 9 months after said administration of said CAR-T cells. In some embodiments, said method is effective in obtaining said progression-free survival at a rate of approximately 75% or less at a follow-up time of 6 months or 9 months after said administration of said CAR-T cells. In some embodiments, said method is effective in obtaining said progression-free survival at a rate of between approximately 29% and approximately 75% at a follow-up time of approximately 6 months or approximately 9 months after said administration of said CAR-T cells. In some embodiments, said method is effective in obtaining said progression-free survival at a rate of approximately 55% at a follow-up time of approximately 6 months or approximately 9 months after said administration of said CAR-T cells.

In some embodiments, the rate of cytokine release syndrome is approximately 60% or greater. In some embodiments, said method is effective in obtaining a rate of cytokine release syndrome of between approximately 60% and approximately 99%. In some embodiments, said method further comprises treating said subject for cytokine release syndrome more than approximately 3 days after said administration of said CAR-T cells.

In some embodiments, the rate of immune-effector cell association neurotoxicity is approximately 20% or greater. In some embodiments, said method is effective in obtaining a rate of immune-effector cell associated neurotoxicity of between approximately 20% and approximately 99%.

### 4.11. Kits and Articles of Manufacture

Any of the compositions described herein may be comprised in a kit. In some embodiments, engineered immortalized CAR-T cells are provided in the kit, which also may include reagents suitable for expanding the cells, such as media.

In a non-limiting example, a chimeric receptor expression construct, one or more reagents to generate a chimeric receptor expression construct, cells for transfection of the expression construct, and/or one or more instruments to obtain immortalized T cells for transfection of the expression construct (such an instrument may be a syringe, pipette, forceps, and/or any such medically approved apparatus).

In some aspects, the kit comprises reagents or apparatuses for electroporation of cells.

In some embodiments, the kit comprises artificial antigen presenting cells.

The kits may comprise one or more suitably aliquoted compositions of the present disclosure or reagents to generate compositions of the disclosure. The components of the kits may be packaged either in aqueous media or in lyophilized form. The container means of the kits may include at least one vial, test tube, flask, bottle, syringe, or other container means, into which a component may he placed, and preferably, suitably aliquoted. Where there is more than one component in the kit, the kit also will generally contain a second, third, or other additional container into which the additional components may be separately placed. However, various combinations of components may be comprised in a vial. The kits of the present disclosure also will typically include a means for containing the chimeric receptor construct and any other reagent containers in close confinement for commercial sale. Such containers may include injection or blow molded plastic containers into which the desired vials are retained, for example.

### 4.12. Particular Embodiments

Particular embodiments of the disclosure are set forth in the following numbered paragraphs:

1. A method of treating a subject, said method comprising administering to the subject a composition comprising a therapeutically effective number of T cells comprising a chimeric antigen receptor (CAR) comprising:
(a) an extracellular antigen binding domain comprising:
   (1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
   (2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain,
to deliver to the subject a dose of CAR expressing T cells (CAR-T cells),
wherein said subject:
   (i) has multiple myeloma
   (ii) has received prior treatment with one, two or three prior lines of therapy; and
   (iii)is lenalidomide-refractory.

2. A method of treating a subject, said method comprising administering to the subject a composition comprising a therapeutically effective number of T cells comprising a chimeric antigen receptor (CAR) comprising:
(a) an extracellular antigen binding domain comprising:
   (1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
   (2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain,
to deliver to the subject a dose of CAR expressing T cells (CAR-T cells),
wherein said subject:
   (i) has multiple myeloma;
   (ii) has received prior treatment with one prior line of therapy, said one prior line of therapy comprising treatment with at least two medicaments, said at least two medicaments comprising a proteasomal inhibitor and an immunomodulatory drug; and
   (iii)has had a prior early relapse.

3. A method of treating a subject, said method comprising administering to the subject a composition comprising a therapeutically effective number of T cells comprising a chimeric antigen receptor (CAR) comprising:
(a) an extracellular antigen binding domain comprising:
   (1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
   (2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain,
to deliver to the subject a dose of CAR expressing T cells (CAR-T cells),
wherein said subject:
   (i) has multiple myeloma; and
   (ii) has received at least one prior line of therapy comprising treatment with at least four medicaments, said at least four medicaments comprising a non-cellular BCMA-targeting medicament.

4. The method of paragraph 1, wherein the subject received prior treatment with at least one prior line of therapy comprising treatment with lenalidomide and at least one non-lenalidomide medicament, said at least one non-lenalidomide medicament comprising at least one of:
(a) a proteasomal inhibitor;
(b) an immunomodulatory drug; or
(c) an anti-CD38 antibody.

5. The method of paragraph 1 or paragraph 4, wherein the subject received prior treatment with at least two prior lines of therapy.

6. The method of paragraph 1, paragraph 4 or paragraph 5, wherein the subject received prior treatment with three prior lines of therapy.

7. The method of any one of paragraphs 1 or 4-6, wherein the subject received prior treatment with dexamethasone, an alkylating agent or daratumumab.

8. The method of any one of paragraphs 1 or 4-7, wherein the multiple myeloma is refractory to the last line of therapy.

9. The method of any one of paragraphs 1 or 4-8, wherein the subject has relapsed after said one, two or three prior lines of therapy.

10. The method of any one of paragraphs 1 or 4-9, wherein the multiple myeloma is refractory to three classes of medicaments.

11. The method of any one of paragraphs 1 or 4-10, wherein said method is effective in obtaining minimal residual disease (MRD) negative status in said subject assessed in the bone marrow after said administration of said CAR-T cells.

12. The method of paragraph 11, wherein said minimal residual disease (MRD) negative status is obtained at a first follow-up time of between approximately 29 days and approximately 184 days after said administration of said CAR-T cells.

13. The method of paragraph 12, wherein said method is effective in maintaining said minimal residual disease (MRD) negative status in said subject assessed in the bone marrow at a second follow-up time of between approximately 57 days and approximately 191 days after said administration of said CAR-T cells, further wherein said first follow-up time is earlier than said second follow-up time.

14. The method of paragraph 11, wherein said method is effective in obtaining said minimal residual disease (MRD) negative status at a rate of between approximately 24% and approximately 61% at a sensitivity threshold level of 10⁻⁴, 10⁻⁵ or 10⁻⁶.

15. The method of paragraph 11, wherein said method is effective in obtaining said minimal residual disease (MRD) negative status at a rate of approximately 41% at a sensitivity threshold level of 10⁻⁴, 10⁻⁵ or 10⁻⁶.

16. The method of paragraph 11, wherein said method is effective in obtaining said minimal residual disease (MRD) negative status at a rate of between approximately 64% and approximately 99% in subjects with evaluable samples at a sensitivity threshold level of 10⁻⁵.

17. The method of paragraph 11, wherein said method is effective in obtaining said minimal residual disease (MRD) negative status at a rate of approximately 92% in subjects with evaluable samples at a sensitivity threshold level of 10⁻⁵.

18. The method of any one of paragraphs 1 or 4-17, wherein said method is effective in obtaining at least one response in the subject after said administration of said CAR-T cells, wherein said at least one response comprises, in order from better to worse:
(i) a stringent complete response;
(ii) a complete response;
(iii) a very good partial response;
(iv) a partial response; or
(v) a minimal response.

19. The method of paragraph 18, wherein said method is effective in obtaining a first response before a time of between approximately 21 days and approximately 99 days after said administration of said CAR-T cells.

20. The method of paragraph 18, wherein said method is effective in obtaining a first response before a time of between approximately 21 days and approximately 55 days after said administration of said CAR-T cells.

21. The method of paragraph 18, wherein said method is effective in obtaining a first response before approximately 36 days after said administration of said CAR-T cells.

22. The method of paragraph 18, wherein said method is effective in obtaining a first response before approximately 30 days after said administration of said CAR-T cells.

23. The method of any one of paragraphs 18-22, wherein said method is effective in obtaining a best response of any one of minimal response, partial response, very good partial response, complete response or stringent complete response.

24. The method of paragraph 23, wherein said method is effective in obtaining said best response of any one of minimal response, partial response, very good partial response, complete response or stringent complete response at a rate of between approximately 52% and approximately 87%.

25. The method of paragraph 23, wherein said method is effective in obtaining said best response of any one of minimal response, partial response, very good partial response, complete response or stringent complete response at a rate of approximately 72%.

26. The method of any one of paragraphs 18-25, wherein said method is effective in obtaining a best response of any one of partial response, very good partial response, complete response or stringent complete response.

27. The method of paragraph 26, wherein said method is effective in obtaining said best response of any one of partial response, very good partial response, complete response or stringent complete response at a rate of between approximately 49% and approximately 84%.

28. The method of paragraph 26, wherein said method is effective in obtaining said best response of any one of partial response, very good partial response, complete response or stringent complete response at a rate of approximately 69%.

29. The method of any one of paragraphs 18-28, wherein said method is effective in obtaining a best response of any one of very good partial response, complete response or stringent complete response.

30. The method of paragraph 29, wherein said method is effective in obtaining said best response of any one of very good partial response, complete response or stringent complete response at a rate of between approximately 49% and approximately 84%.

31. The method of paragraph 29, wherein said method is effective in obtaining said best response of any one of very good partial response, complete response or stringent complete response at a rate of approximately 69%.

32. The method of any one of paragraphs 18-31, wherein said method is effective in obtaining a best response of complete response or stringent complete response.

33. The method of paragraph 32, wherein said method is effective in obtaining said best response of complete response or stringent complete response at a rate of between approximately 39% and approximately 76%.

34. The method of paragraph 32, wherein said method is effective in obtaining said best response of complete response or stringent complete response at a rate of approximately 58%.

35. The method of any one of paragraphs 18-34, wherein said method is effective in obtaining a best response of stringent complete response.

36. The method of paragraph 35, wherein said method is effective in obtaining said best response of stringent complete response at a rate of between approximately 33% and approximately 70%.

37. The method of paragraph 35, wherein said method is effective in obtaining said best response of stringent complete response at a rate of approximately 52%.

38. The method of any one of paragraphs 1 or 4-37, wherein said method is effective in obtaining progression-free survival of the subject.

39. The method of paragraph 38, wherein said method is effective in obtaining said progression-free survival of the subject at a time between said administration of said CAR-T cells and approximately 55 days after said administration of said CAR-T cells.

40. The method of paragraph 38, wherein said method is effective in obtaining said progression-free survival of the subject at a time between said administration of said CAR-T cells and approximately 297 days after said administration of said CAR-T cells.

41. The method of paragraph 38, wherein said method is effective in obtaining said progression-free survival at a rate of between approximately 62% and approximately 95% at a follow-up time of approximately 6 months or approximately 9 months after said administration of said CAR-T cells.

42. The method of paragraph 38, wherein said method is effective in obtaining said progression-free survival at a rate of approximately 86% at a follow-up time of approximately 6 months or approximately 9 months after said administration of said CAR-T cells.

43. The method of any one of paragraphs 1 or 4-42, wherein said method further comprises treating said subject for cytokine release syndrome more than approximately 3 days after said administration of said CAR-T cells.

44. The method of paragraph 43, wherein said method is effective in obtaining a rate of recovery from said cytokine release syndrome of between approximately 1% and approximately 90% at a time of approximately 7 days after first observance of said cytokine release syndrome.

45. The method of any one of paragraphs 1 or 4-44, wherein said method is effective in obtaining a rate of immune-effector cell associated neurotoxicity of between approximately 20% and approximately 99%.

46. The method of any one of paragraphs 23-45, wherein said method is effective in obtaining said best response before a time of between approximately 27 days and approximately 237 days after said administration of said CAR-T cells.

47. The method of any one of paragraphs 23-45, wherein said method is effective in obtaining said best response before a time of between approximately 46 days and approximately 172 days after said administration of said CAR-T cells.

48. The method of any one of paragraphs 23-45, wherein said method is effective in obtaining said best response before approximately 109 days after said administration of said CAR-T cells.

49. The method of any one of paragraphs 23-45, wherein said method is effective in obtaining said best response before approximately 87 days after said administration of said CAR-T cells.

50. The method of any one of paragraphs 19-49, wherein said method is effective in maintaining a response in the subject at a follow-up time between the time of said first response and approximately 270 days after said administration of said CAR-T cells.

51. The method of any one of paragraphs 18-50, wherein said method is effective in maintaining a response at a rate of between approximately 70% and approximately 99% at a follow-up time of approximately 6 months after said administration of said CAR-T cells.

52. The method of any one of paragraphs 18-50, wherein said method is effective in maintaining a response at a rate of approximately 95% at a follow-up time of approximately 6 months after said administration of said CAR-T cells.

53. The method of any one of paragraphs 18-50, wherein said method is effective in maintaining a response at a rate of between approximately 7% and approximately 92% at a follow-up time of approximately 9 months after said administration of said CAR-T cells.

54. The method of any one of paragraphs 18-50, wherein said method is effective in maintaining a response at a rate of approximately 63% at a follow-up time of approximately 9 months after said administration of said CAR-T cells.

55. The method of any one of paragraphs 32-54, further wherein said method is effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells.

56. The method of paragraph 55, wherein said method is effective in obtaining either minimal residual disease (MRD) negative complete response or minimal residual disease (MRD) negative stringent complete response at a rate of between approximately 18% and approximately 54% at a follow-up time of approximately 291 days after said administration of said CAR-T cells.

57. The method of paragraph 55, wherein said method is effective in obtaining either minimal residual disease (MRD) negative complete response or minimal residual disease (MRD) negative stringent complete response at a rate of approximately 35% at a follow-up time of approximately 291 days after said administration of said CAR-T cells.

58. The method of paragraph 2, wherein the subject was additionally treated with an anti-CD38 antibody.

59. The method of paragraph 2 or paragraph 58, wherein the multiple myeloma is refractory to at least one medicament.

60. The method of any one of paragraphs 2 or 58-59, wherein said method is effective in obtaining minimal residual disease (MRD) negative status in said subject assessed in the bone marrow after said administration of said CAR-T cells.

61. The method of paragraph 60, wherein said minimal residual disease (MRD) negative status is assessed in the bone marrow at a first follow-up time at a first follow-up time of between approximately 35 days and approximately 58 days after said administration of said CAR-T cells.

62. The method of paragraph 61, wherein said method is effective in maintaining said minimal residual disease (MRD) negative status in said subject assessed in the bone marrow at a second follow-up time of between approximately 78 days and approximately 359 days after said administration of said CAR-T cells, further wherein said first follow-up time is earlier than said second follow-up time.

63. The method of paragraph 60, wherein said method is effective in obtaining said minimal residual disease (MRD) negative status at a rate of between approximately 26% and approximately 74% at a sensitivity threshold level of 10⁻⁴ or 10⁻⁵ or at a rate of between approximately 17% and approximately 64% at a sensitivity threshold level of 10⁻⁶.

64. The method of paragraph 60, wherein said method is effective in obtaining said minimal residual disease (MRD) negative status at a rate of approximately 50% at a sensitivity threshold level of 10⁻⁴ or 10⁻⁵ or at a rate of approximately 39% at a sensitivity threshold level of 10⁻⁶.

65. The method of paragraph 60, wherein said method is effective in obtaining said minimal residual disease (MRD) negative status at a rate of between approximately 66% and approximately 100% in subjects with evaluable samples at a sensitivity threshold level of 10⁻⁵.

66. The method of paragraph 60, wherein said method is effective in obtaining said minimal residual disease (MRD) negative status at a rate of approximately 100% in subjects with evaluable samples at a sensitivity threshold level of 10⁻⁵.

67. The method of any one of paragraphs 2 or 58-66, wherein said method is effective in obtaining at least one response in the subject after said administration of said CAR-T cells, wherein said at least one response comprises, in order from better to worse:
(i) a stringent complete response;
(ii) a complete response;
(iii) a very good partial response;
(iv) a partial response; or
(v) a minimal response.

68. The method of paragraph 67, wherein said method is effective in obtaining a first response before a time of between approximately 27 days and approximately 78 days after said administration of said CAR-T cells.

69. The method of paragraph 67, wherein said method is effective in obtaining a first response before a time of between approximately 27 days and approximately 47 days after said administration of said CAR-T cells.

70. The method of paragraph 67, wherein said method is effective in obtaining a first response before approximately 33 days after said administration of said CAR-T cells.

71. The method of paragraph 67, wherein said method is effective in obtaining a first response before approximately 28 days after said administration of said CAR-T cells.

72. The method of any one of paragraphs 67-71, wherein said method is effective in obtaining a best response of any one of minimal response, partial response, very good partial response, complete response or stringent complete response.

73. The method of paragraph 72, wherein said method is effective in obtaining said best response of any one of minimal response, partial response, very good partial response, complete response or stringent complete response at a rate of between approximately 65% and approximately 99%.

74. The method of paragraph 72, wherein said method is effective in obtaining said best response of any one of minimal response, partial response, very good partial response, complete response or stringent complete response at a rate of approximately 89%.

75. The method of any one of paragraphs 67-74, wherein said method is effective in obtaining a best response of any one of partial response, very good partial response, complete response or stringent complete response.

76. The method of paragraph 75, wherein said method is effective in obtaining said best response of any one of partial response, very good partial response, complete response or stringent complete response at a rate of between approximately 65% and approximately 99%.

77. The method of paragraph 75, wherein said method is effective in obtaining said best response of any one of partial response, very good partial response, complete response or stringent complete response at a rate of approximately 89%.

78. The method of any one of paragraphs 67-77, wherein said method is effective in obtaining a best response of any one of very good partial response, complete response or stringent complete response.

79. The method of paragraph 78, wherein said method is effective in obtaining said best response of any one of very good partial response, complete response or stringent complete response at a rate of between approximately 41% and approximately 87%.

80. The method of paragraph 78, wherein said method is effective in obtaining said best response of any one of very good partial response, complete response or stringent complete response at a rate of approximately 67%.

81. The method of any one of paragraphs 67-80, wherein said method is effective in obtaining a best response of complete response or stringent complete response.

82. The method of paragraph 81, wherein said method is effective in obtaining said best response of complete response or stringent complete response at a rate of between and approximately 10% and approximately 54%.

83. The method of paragraph 81, wherein said method is effective in obtaining said best response of complete response or stringent complete response at a rate of approximately 28%.

84. The method of any one of paragraphs 67-83, wherein said method is effective in obtaining a best response of stringent complete response.

85. The method of paragraph 84, wherein said method is effective in obtaining said best response of stringent complete response at a rate of between approximately 6% and approximately 48%.

86. The method of paragraph 84, wherein said method is effective in obtaining said best response of stringent complete response at a rate of approximately 22%.

87. The method of any one of paragraphs 2 or 58-86, wherein said method is effective in obtaining progression-free survival of the subject.

88. The method of paragraph 87, wherein said method is effective in obtaining said progression-free survival of the subject at a time between said administration of said CAR-T cells and approximately 182 days after said administration of said CAR-T cells.

89. The method of paragraph 87, wherein said method is effective in obtaining said progression-free survival at a rate of approximately 100% at a follow-up time of approximately 6 months after said administration of said CAR-T cells.

90. The method of paragraph 87, wherein said method is effective in obtaining said progression-free survival at a rate of between approximately 5% and approximately 95% at a follow-up time of approximately 9 months or approximately 12 months after said administration of said CAR-T cells.

91. The method of paragraph 87, wherein said method is effective in obtaining said progression-free survival at a rate of approximately 67% at a follow-up time of approximately 9 months or approximately 12 months after said administration of said CAR-T cells.

92. The method of any one of paragraphs 2 or 58-91, wherein said method further comprises treating said subject for cytokine release syndrome more than approximately 3 days after said administration of said CAR-T cells.

93. The method of paragraph 92, wherein said method is effective obtaining a rate of recovery from said cytokine release syndrome of between approximately 1% and approximately 100% at a time of approximately 7 days after first observance of said cytokine release syndrome.

94. The method of any one of paragraphs 72-93, wherein said method is effective in obtaining said best response before a time of between approximately 27 days and approximately 354 days after said administration of said CAR-T cells.

95. The method of any one of paragraphs 72-93, wherein said method is effective in obtaining said best response before a time of between approximately 27 days and approximately 155 days after said administration of said CAR-T cells.

96. The method of any one of paragraphs 72-93, wherein said method is effective in obtaining said best response before approximately 71 days after said administration of said CAR-T cells.

97. The method of any one of paragraphs 72-93, wherein said method is effective in obtaining said best response before approximately 42 days after said administration of said CAR-T cells.

98. The method of any one of paragraphs 68-97, wherein said method is effective in maintaining a response in the subject at a follow-up time of between the time of said first response and approximately 156 days after said administration of said CAR-T cells.

99. The method of any one of paragraphs 68-98, wherein said method is effective in maintaining a response at a rate of between approximately 5% and approximately 95% at a follow-up time of approximately 6 months, approximately 9 months or approximately 12 months after said administration of said CAR-T cells.

100. The method of any one of paragraphs 68-98, wherein said method is effective in maintaining a response at a rate of approximately 67% at a follow-up time of approximately 6 months, approximately 9 months or approximately 12 months after said administration of said CAR-T cells.

101. The method of any one of paragraphs 81-100, further wherein said method is effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells.

102. The method of paragraph 101, wherein said method is effective in obtaining either minimal residual disease (MRD) negative complete response or minimal residual disease (MRD) negative stringent complete response at a rate of between approximately 1% and approximately 35% at a follow-up time of approximately 141 days after said administration of said CAR-T cells.

103. The method of paragraph 101, wherein said method is effective in obtaining either minimal residual disease (MRD) negative complete response or minimal residual disease (MRD) negative stringent complete response at a rate of approximately 11% at a follow-up time of approximately 141 days after said administration of said CAR-T cells.

104. The method of paragraph 3, wherein the subject received prior treatment with at least two prior lines of therapy.

105. The method of paragraph 3 or paragraph 104, wherein the subject received prior treatment with at least four prior lines of therapy.

106. The method of any one of paragraphs 3 or 104-105, wherein the subject received prior treatment with at least eight prior lines of therapy.

107. The method of any one of paragraphs 3 or 104-106, wherein the subject received prior treatment with at least twelve prior lines of therapy.

108. The method of any one of paragraphs 3 or 104-107, wherein the subject has relapsed after said at least one prior line of therapy.

109. The method of any one of paragraphs 3 or 104-108, wherein said method is effective in obtaining minimal residual disease (MRD) negative status in said subject assessed in the bone marrow after said administration of said CAR-T cells.

110. The method of paragraph 109, wherein said minimal residual disease (MRD) negative status is assessed in the bone marrow at a first follow-up time at a first follow-up time of between approximately 56 days and approximately 58 days after said administration of said CAR-T cells.

111. The method of paragraph 110, wherein said method is effective in maintaining said minimal residual disease (MRD) negative status in said subject assessed in the bone marrow at a second follow-up time of between approximately 183 days and approximately 186 days after said administration of said CAR-T cells, further wherein said first follow-up time is earlier than said second follow-up time.

112. The method of paragraph 109, wherein said method is effective in obtaining said minimal residual disease (MRD) negative status at a rate of between approximately 9% and approximately 49% at a sensitivity threshold level of 10⁻⁴, at a rate of between approximately 6% and approximately 44% at a sensitivity threshold level of 10⁻⁵, or at a rate of between approximately 1% and approximately 31% at a sensitivity threshold level of 10⁻⁶.

113. The method of paragraph 109, wherein said method is effective in obtaining said minimal residual disease (MRD) negative status at a rate of approximately 25% at a sensitivity threshold level of 10⁻⁴, at a rate of approximately 20% at a sensitivity threshold level of 10⁻⁵, or at a rate of approximately 10% at a sensitivity threshold level of 10⁻⁶.

114. The method of paragraph 109, wherein said method is effective in obtaining said minimal residual disease (MRD) negative status at a rate of between approximately 22% and approximately 96% in subjects with evaluable samples at a sensitivity threshold level of 10⁻⁵.

115. The method of paragraph 109, wherein said method is effective in obtaining said minimal residual disease (MRD) negative status at a rate of approximately 67% in subjects with evaluable samples at a sensitivity threshold level of 10⁻⁵.

116. The method of any one of paragraphs 3 or 104-115, wherein said method is effective in obtaining at least one response in the subject after said administration of said CAR-T cells, wherein said at least one response comprises, in order from better to worse:
(i) a stringent complete response;
(ii) a complete response;
(iii) a very good partial response;
(iv) a partial response; or
(v) a minimal response.

117. The method of paragraph 116, wherein said method is effective in obtaining a first response before a time of between approximately 27 days and approximately 153 days after said administration of said CAR-T cells.

118. The method of paragraph 116, wherein said method is effective in obtaining a first response before a time of between approximately 27 days and approximately 88 days after said administration of said CAR-T cells.

119. The method of paragraph 116, wherein said method is effective in obtaining a first response before approximately 43 days after said administration of said CAR-T cells.

120. The method of paragraph 116, wherein said method is effective in obtaining a first response before approximately 28 days after said administration of said CAR-T cells.

121. The method of any one of paragraphs 116-120, wherein said method is effective in obtaining a best response of any one of minimal response, partial response, very good partial response, complete response or stringent complete response.

122. The method of paragraph 121, wherein said method is effective in obtaining said best response of any one of minimal response, partial response, very good partial response, complete response or stringent complete response at a rate of between approximately 23% and approximately 69%.

123. The method of paragraph 121, wherein said method is effective in obtaining said best response of any one of minimal response, partial response, very good partial response, complete response or stringent complete response at a rate of approximately 45%.

124. The method of any one of paragraphs 116-123, wherein said method is effective in obtaining a best response of any one of partial response, very good partial response, complete response or stringent complete response.

125. The method of paragraph 124, wherein said method is effective in obtaining said best response of any one of partial response, very good partial response, complete response or stringent complete response at a rate of between approximately 19% and approximately 64%.

126. The method of paragraph 124, wherein said method is effective in obtaining said best response of any one of partial response, very good partial response, complete response or stringent complete response at a rate of approximately 40%.

127. The method of any one of paragraphs 116-126, wherein said method is effective in obtaining a best response of any one of very good partial response, complete response or stringent complete response.

128. The method of paragraph 127, wherein said method is effective in obtaining said best response of any one of very good partial response, complete response or stringent complete response at a rate of between approximately 15% and approximately 59%.

129. The method of paragraph 127, wherein said method is effective in obtaining said best response of any one of very good partial response, complete response or stringent complete response at a rate of approximately 35%.

130. The method of any one of paragraphs 116-129, wherein said method is effective in obtaining a best response of complete response or stringent complete response.

131. The method of paragraph 130, wherein said method is effective in obtaining said best response of complete response or stringent complete response at a rate of between approximately 3% and approximately 38%.

132. The method of any one of paragraphs 116-131, wherein said method is effective in obtaining a best response of stringent complete response.

133. The method of paragraph 132, wherein said method is effective in obtaining said best response of stringent complete response at a rate of between approximately 1% and approximately 32%.

134. The method of any one of paragraphs 3 or 104-133, wherein said method is effective in obtaining progression-free survival of the subject.

135. The method of paragraph 134, wherein said method is effective in obtaining said progression-free survival of the subject at a time between said administration of said CAR-T cells and approximately 15 days after said administration of said CAR-T cells.

136. The method of paragraph 134, wherein said method is effective in obtaining said progression-free survival of the subject at a time between said administration of said CAR-T cells and approximately 44 days after said administration of said CAR-T cells.

137. The method of paragraph 134, wherein said method is effective in obtaining said progression-free survival of the subject at a time between said administration of said CAR-T cells and approximately 159 days after said administration of said CAR-T cells.

138. The method of paragraph 134, wherein said method is effective in obtaining said progression-free survival at a rate of between approximately 29% and approximately 75% at a follow-up time of approximately 6 months or approximately 9 months after said administration of said CAR-T cells.

139. The method of paragraph 134, wherein said method is effective in obtaining said progression-free survival at a rate of approximately 55% at a follow-up time of approximately 6 months or approximately 9 months after said administration of said CAR-T cells.

140. The method of any one of paragraphs 3 or 104-139, wherein said method is effective in obtaining a rate of cytokine release syndrome of between approximately 60% and approximately 99%.

141. The method of paragraph 140, wherein said method further comprises treating said subject for cytokine release syndrome more than approximately 3 days after said administration of said CAR-T cells.

142. The method of any one of paragraphs 3 or 104-141, wherein said method is effective in obtaining a rate of immune-effector cell associated neurotoxicity of between approximately 20% and approximately 99%.

143. The method of any one of paragraphs 3 or 104-142, wherein said at least four medicaments further comprises a proteasomal inhibitor, an immunomodulatory drug and an anti-CD38 antibody.

144. The method of any one of paragraphs 121-143, wherein said method is effective in obtaining said best response before a time of between approximately 27 days and approximately 171 days after said administration of said CAR-T cells.

145. The method of any one of paragraphs 121-143, wherein said method is effective in obtaining said best response before a time of between approximately 27 days and approximately 133 days after said administration of said CAR-T cells.

146. The method of any one of paragraphs 121-143, wherein said method is effective in obtaining said best response before approximately 78 days after said administration of said CAR-T cells.

147. The method of any one of paragraphs 121-143, wherein said method is effective in obtaining said best response before approximately 56 days after said administration of said CAR-T cells.

148. The method of any one of paragraphs 117-147, wherein said method is effective in maintaining a response in the subject at a follow-up time of between the time of said first response and approximately 132 days after said administration of said CAR-T cells, further wherein said first response was obtained between the time of said administration of said CAR-T cells and approximately 131 days after said administration of said CAR-T cells.

149. The method of any one of paragraphs 117-147, wherein said method is effective in maintaining a response at a rate of between approximately 20% and approximately 96% at a follow-up time of approximately 6 months after said administration of said CAR-T cells.

150. The method of any one of paragraphs 117-147, wherein said method is effective in maintaining a response at a rate of approximately 80% at a follow-up time of approximately 6 months after said administration of said CAR-T cells.

151. The method of any one of paragraphs 1-150, wherein the multiple myeloma is refractory to at least two medicaments.

152. The method of any one of paragraphs 1-151, wherein the multiple myeloma is refractory to at least three medicaments.

153. The method of any one of paragraphs 1-152, wherein the multiple myeloma is refractory to at least four medicaments.

154. The method of any one of paragraphs 1-153, wherein the multiple myeloma is refractory to at least five medicaments.

155. The method of any one of paragraphs 1-154, wherein the subject has bone marrow plasma cells of between approximately 10% and approximately 30% before said administration of said CAR-T cells.

156. The method of any one of paragraphs 1-155, wherein the dose comprises 1.0 × 10⁵ to 5.0 × 10⁶ of said CAR-T cells per kilogram of the mass of the subject.

157. The method of any one of paragraphs 1-156, wherein the dose comprises 5.0 × 10⁵ to 1.0 × 10⁶ of said CAR-T cells per kilogram of the mass of the subject.

158. The method of any one of paragraphs 1-157, wherein the dose comprises approximately 0.75 × 10⁶ of said CAR-T cells per kilogram of the mass of the subject.

159. The method of any one of paragraphs 1-158, wherein the dose comprises less than 1.0 x 10⁸ of said CAR-T cells per subject.

160. The method of any one of paragraphs 1-159, wherein said administration of said CAR-T cells is via a single intravenous infusion.

161. The method of paragraph 160, wherein said single intravenous infusion is administered using a single bag of said CAR-T cells.

162. The method of paragraph 161, wherein said administration of said single bag of said CAR-T cells is completed between the time at which said single bag of CAR-T cells is thawed and three hours after said single bag of CAR-T cells is thawed.

163. The method of paragraph 160, wherein said single intravenous administration is administered using two bags of said CAR-T cells.

164. The method of paragraph 163, wherein said administration of each of said two bags of said CAR-T cells is completed between the time at which a first bag of said two bags of CAR-T cells is thawed and three hours after said first bag of CAR-T cells is thawed.

165. The method of any one of paragraphs 1-164, wherein a lymphodepleting regimen precedes said administration of CAR-T cells by approximately 5 days to approximately 7 days.

166. The method of paragraph 165, wherein said lymphodepleting regimen is administered intravenously.

167. The method of paragraph 166, wherein said lymphodepleting regimen comprises:
(a) administration of cyclophosphamide; or
(b) administration of fludarabine.

168. The method of paragraph 167, wherein said cyclophosphamide is administered intravenously at 300 mg/m².

169. The method of paragraph 167, wherein said fludarabine is administered intravenously at 30 mg/m².

170. The method of any one of paragraphs 1-169, wherein a lymphodepleting regimen comprising cyclophosphamide administered intravenously at 300 mg/m² and fludarabine administered intravenously at 30 mg/m² precedes said administration of CAR-T cells by approximately 5 days to approximately 7 days.

171. The method of any one of paragraph 165-170, wherein the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject.

172. The method of any paragraph 171, wherein the subject had an increase in tumor burden despite said bridging therapy.

173. The method of any paragraph 171, wherein the subject had an increase in tumor burden of approximately 25% or greater despite said bridging therapy.

174. The method of any one of paragraphs 43-44, 92-93 or 141, wherein said method further comprises treating said subject for cytokine release syndrome more than approximately 3 days following said administration of said CAR-T cells without significantly reducing expansion of said CAR-T cells in vivo.

175. The method of any one of paragraphs 43-44, 92-93, 141 or 174, wherein said treatment of cytokine release syndrome comprises administering an IL-6R inhibitor to the subject.

176. The method of paragraph 175, wherein said IL-6R inhibitor is an antibody.

177. The method of paragraph 176, wherein said antibody inhibits IL-6R by binding its extracellular domain.

178. The method of any one of paragraphs 175-177, wherein said IL-6R inhibitor prevents the binding of IL-6 to IL-6R.

179. The method of any one of paragraphs 175-178, wherein the IL-6R inhibitor is tocilizumab.

180. The method of any one of paragraphs 1-179, wherein the subject is treated with pre-administration medication comprising an antipyretic and an antihistamine up to approximately 1 hour before said administration of said CAR-T cells.

181. The method of paragraph 180, wherein said antipyretic comprises either paracetamol or acetaminophen.

182. The method of paragraph 180, wherein said antipyretic is administered to the subject either orally or intravenously.

183. The method of paragraph 180, wherein said antipyretic is administered to the subject at a dosage of between 650 mg and 1000 mg.

184. The method of paragraph 180, wherein said antihistamine comprises diphenhydramine.

185. The method of paragraph 180, wherein said antihistamine is administered to the subject either orally or intravenously.

186. The method of paragraph 180, wherein said antihistamine is administered at a dosage of between 25 mg and 50 mg, or its equivalent.

187. The method of paragraph 180, wherein said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent.

188. The method of any one of paragraphs 1-187, wherein the composition comprising CAR-T cells administered to the subject further comprises an excipient selected from dimethylsulfoxide or dextran-40.

189. The method of any one of paragraphs 1-188, wherein the first BCMA binding moiety and/or the second BCMA binding moiety is an anti-BCMA VHH.

190. The method of paragraph 189, wherein the first BCMA binding moiety is a first anti-BCMA VHH and the second BCMA binding moiety is a second anti-BCMA VHH.

191. The method of any one of paragraphs 1-190, wherein the first BCMA binding moiety comprises the amino acid sequence of SEQ ID NO: 2.

192. The method of any one of paragraphs 1-191, wherein the first BCMA binding moiety comprises a polypeptide encoded by the nucleic acid sequence of SEQ ID NO: 10.

193. The method of any one of paragraphs 1-192, wherein the second BCMA binding moiety comprises the amino acid sequence of SEQ ID NO: 4.

194. The method of any one of paragraphs 1-193, wherein the second BCMA binding moiety comprises a polypeptide encoded by the nucleic acid sequence of SEQ ID NO: 12.

195. The method of any one of paragraphs 1-194, wherein the first BCMA binding moiety and the second BCMA binding moiety are connected to each other via a peptide linker.

196. The method of paragraph 195, wherein the peptide linker comprises the amino acid sequence of SEQ ID NO: 3.

197. The method of paragraph 195, wherein the peptide linker comprises a polypeptide encoded by the nucleic acid sequence of SEQ ID NO: 11.

198. The method of any one of paragraphs 1-197, wherein the CAR polypeptide further comprises a signal peptide located at the N-terminus of the polypeptide.

199. The method of paragraph 198, wherein the signal peptide is derived from CD8-alpha.

200. The method of paragraph 198, wherein the signal peptide comprises the amino acid sequence of SEQ ID NO: 1.

201. The method of paragraph 198, wherein the signal peptide comprises a polypeptide encoded by the nucleic acid sequence of SEQ ID NO: 9.

202. The method of any one of paragraphs 1-201, wherein the transmembrane domain comprises the amino acid sequence of SEQ ID NO: 6.

203. The method of any one of paragraphs 1-202, wherein the transmembrane domain comprises a polypeptide encoded by the nucleic acid sequence of SEQ ID NO: 14.

204. The method of any one of paragraphs 1-203, wherein the intracellular signaling domain comprises a primary intracellular signaling domain of an immune effector cell.

205. The method of any one of paragraphs 1-204, wherein the intracellular signaling domain is derived from CD3ζ.

206. The method of any one of paragraphs 1-205, wherein the intracellular signaling domain comprises at least one co-stimulatory signaling domains.

207. The method of any one of paragraphs 1-206, wherein the intracellular signaling domain comprises the amino acid sequence of SEQ ID NO: 8.

208. The method of any one of paragraphs 1-207, wherein the intracellular signaling domain comprises a polypeptide encoded by the nucleic acid sequence of SEQ ID NO: 16.

209. The method of any one of paragraphs 1-208, wherein the intracellular signaling domain comprises an amino acid sequence of SEQ ID NO: 7.

210. The method of any one of paragraphs 1-209, wherein the intracellular signaling domain comprises a polypeptide encoded by the nucleic acid sequence of SEQ ID NO: 15.

211. The method of any one of paragraphs 1-210, wherein the CAR polypeptide further comprises a hinge domain located between the C-terminus of the extracellular antigen binding domain and the N-terminus of the transmembrane domain.

212. The method of paragraph 211, wherein the hinge domain comprises the amino acid sequence of SEQ ID NO: 5.

213. The method of paragraph 211, wherein the hinge domain comprises a polypeptide encoded by the nucleic acid sequence of SEQ ID NO: 13.

214. The method of any one of paragraphs 1-213, wherein the CAR comprises the amino acid sequence of SEQ ID NO: 17.

215. The method of any one of paragraphs 1-214, wherein the T cells are autologous T cells.

216. The method of any one of paragraphs 1-215, wherein the T cells are allogeneic T cells.

217. The method of any one of paragraphs 1-216, wherein the subject is human.

218. The method of any one of paragraphs 1-103, wherein the subject has had no prior exposure to a BCMA-targeting medicament.

219. The method of any one of paragraphs 1-218, wherein the multiple myeloma is progressive.

### 5. EXAMPLES

The following examples are provided to further describe some of the embodiments disclosed herein. The examples are intended to illustrate, not to limit, the disclosed embodiments.

### 5.1. Example 1: Ciltacabtagene Autoleucel

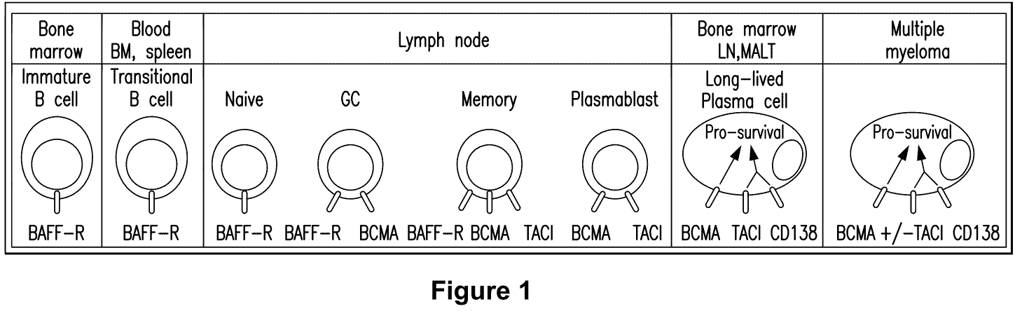

B cell maturation antigen (BCMA, also known as CD269 and TNFRSF17) is a 20 kilodalton, type III membrane protein that is part of the tumor necrosis receptor superfamily. BCMA is a cell surface antigen that is predominantly expressed in B-lineage cells at high levels. FIG. 1 shows the expression of BCMA on various immune-derived cells. Comparative studies have shown a lack of BCMA in most normal tissues and absence of expression on CD34-positive hematopoietic stem cells. BCMA binds 2 ligands that induce B cell proliferation, and plays a critical role in B cell maturation and subsequent differentiation into plasma cells. The selective expression and the biological importance for the proliferation and survival of myeloma cells makes BCMA a promising target for CAR-T based immunotherapy, ciltacabtagene autoleucel.

Ciltacabtagene autoleucel is an autologous chimeric antigen receptor T cell (CAR-T) therapy that targets BCMA. The ciltacabtagene autoleucel chimeric antigen receptor (CAR) comprises two B-cell maturation antigen (BCMA)-targeting VHH domains designed to confer avidity. A map of the construct is depicted in FIG. 2.

### 5.2. Example 2: Method of Treating Cohort A, Cohort B and Cohort C with Ciltacabtagene Autoleucel

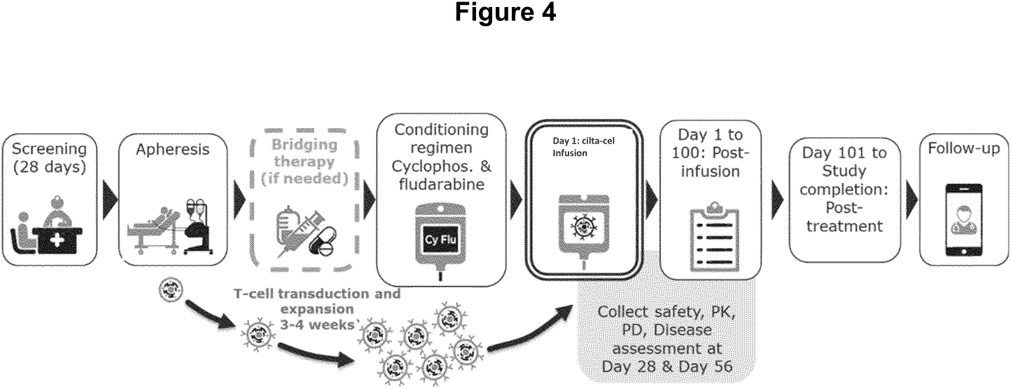

In the multicohort, open-label, phase 2 study, we evaluated cilta-cel safety and efficacy in various clinical settings for patients with multiple myeloma who are in dire need of therapy and have an overall survival rate of no greater than about 50%. A schematic overview of the study flow chart, which consists of a lymphodepleting regimen prior to cilta-cel infusion, is depicted in FIG. 4.

Eligible subjects underwent apheresis for collection of peripheral blood mononuclear cells (PBMC). Study enrollment was defined at the day of apheresis. The ciltacabtagene autoleucel drug product (DP) was generated from T cells selected from the apheresis. Subjects for whom apheresis or manufacturing failed were allowed a second attempt at apheresis.

Bridging therapy (anti-plasma cell directed treatment between apheresis and the first dose of the conditioning regimen) was allowed when clinically indicated (i.e., to maintain disease stability while waiting for manufacturing of ciltacabtagene autoleucel). Additional cycles of bridging therapy were considered based on the subject’s clinical status and timing of availability of CAR-T product. A bridging therapy is defined as short-term treatment which had previously generated at least a response of stable disease for the subject.

After meeting safety criteria for treatment, subjects were administered a conditioning regimen to help achieve to lymphodepletion and promote CAR-T cell expansion in the subject. The lymphodepleting regimen comprised intravenous (IV) administration of cyclophosphamide 300 mg/m² and fludarabine 30 mg/m² daily for 3 days. Cyclophosphamide 300 mg/m² and fludarabine 30 mg/m² before cilta-cel infusion is consistent with the lymphodepletion regimen used in the marketed CAR-T products Kymriah and Yescarta.

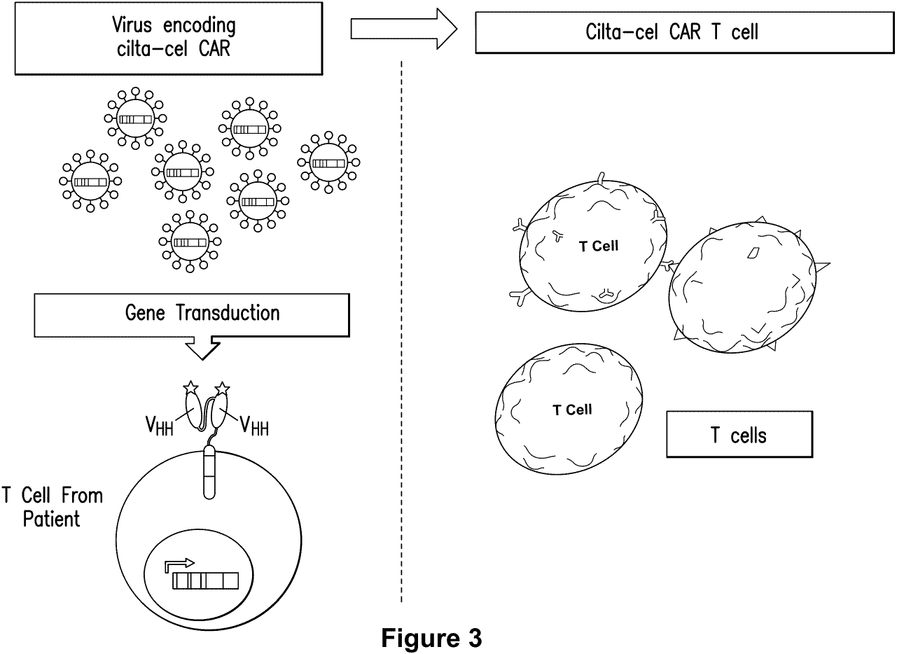

5 to 7 days after start of the conditioning regimen, cilta-cel, which had been prepared from apheresed material via viral transduction as shown in FIG. 3, was administered on a day defined as Day 1. Approximately one hour prior to cilta-cel infusion, subjects received premedication. Corticosteroids were not be used during pre-infusion. Pre-infusion medication is listed in Table 1. Following treatment with the pre-infusion medication, cilta-cel administration was performed in a single infusion at a total targeted dose of 0.75 × 10⁶ CAR-positive viable T cells/kg (range: 0.5-1.0 × 10⁶ CAR-positive viable T cells/kg) with a maximum total dose of 1.0 × 10⁸ CAR-positive viable T cells.

A dose of ciltacabtagene autoleucel was contained in either 1 or 2 cryopreserved patient-specific infusion bags. The timing of cilta-cel thaw was coordinated with the timing of the infusion. The infusion time was confirmed in advance, and the start time for thaw was adjusted so that cilta-cel was available for infusion when the patient would have been ready. If more than one bag was received for the treatment infusion, 1 bag was thawed at a time. The thawing/infusion of the next bag was made to wait until it was determined that the previous bag had been safely administered.

### 5.3. Example 3: Evaluation of Efficacy of Method of Treating Cohort A, Cohort B and Cohort C With Ciltacabtagene Autoleucel

Using the IMWG-based response criteria summarized in Table 2, this study classified a response, in order from better to worse, as either a stringent complete response (sCR), a complete response (CR), a very good partial response (VGPR), a partial response (PR), a minimal response (MR), a stable disease or a progressive disease. Disease progression was consistently documented across clinical study sites. The tests performed to assess IMWG-based response criteria are as follows:
Myeloma Protein Measurements in Serum and Urine: Myeloma protein (M-protein) measurements were made using the following tests from blood and 24-hour urine samples: serum quantitative Ig, serum protein electrophoresis (SPEP), serum immunofixation electrophoresis, serum FLC assay (for subject in suspected CR/sCR and every disease assessment for subjects with serum FLC only disease), 24-hour urine M-protein quantitation by electrophoresis (UPEP), urine immunofixation electrophoresis, serum β2-microglobulin. Disease progression based on one of the laboratory tests alone were confirmed by at least 1 repeat investigation. Disease evaluations continued beyond relapse from CR until disease progression was confirmed. Serum and urine immunofixation and serum free light chain (FLC) assays were performed at screening and thereafter when a CR was suspected (when serum or 24-hour urine M-protein electrophoresis [by SPEP or UPEP] were 0 or non-quantifiable). For subjects with light chain multiple myeloma, serum and urine immunofixation tests were performed routinely.
Serum Calcium Corrected for Albumin: Blood samples for calculating serum calcium corrected for albumin were collected and analyzed until the development of confirmed disease progression; development of hypercalcemia (corrected serum calcium >11.5 mg/dL [>2.9 mmol/L]) may indicate disease progression or relapse if it is not attributable to any other cause. Calcium binds to albumin and only the unbound (free) calcium is biologically active; therefore, the serum calcium level must be adjusted for abnormal albumin levels (“corrected serum calcium”).
Bone Marrow Examination: Bone marrow aspirate or biopsy was performed for clinical assessments. Bone marrow aspirate was performed for biomarker evaluations. Clinical staging (morphology, cytogenetics, and immunohistochemistry or immunofluorescence or flow cytometry) was done. A portion of the bone marrow aspirate was immunophenotyped and monitor for BCMA, checkpoint ligand expression in CD138-positive multiple myeloma cells, and checkpoint expression on T cells. If feasible, bone marrow aspirate also was performed to confirm CR and sCR and at disease progression. Additionally, since minimal residual disease (MRD) negativity was being evaluated as a potential surrogate for PFS and OS in multiple myeloma treatment, MRD was monitored in subjects using next generation sequencing (NGS) on bone marrow aspirate DNA. Baseline bone marrow aspirates were used to define the myeloma clones, and post-treatment samples were used to evaluate MRD negativity. A fresh bone marrow aspirate was collected prior to the first dose of conditioning regimen (≤7 days).
Skeletal Survey: A skeletal survey (including skull, entire vertebral column, pelvis, chest, humeri, femora, and any other bones for which the investigator suspects involvement by disease) was performed during the screening phase and evaluated by either roentgenography (“X-rays”) or low-dose computed tomography (CT) scans without the use of IV contrast. If a CT scan was used, it was of diagnostic quality. Following cilta-cel infusion, and before disease progression was confirmed, X-rays or CT scans were performed locally, whenever clinically indicated based on symptoms, to document response or progression. Magnetic resonance imaging (MRI) was an acceptable method for evaluation of bone disease, and was included at discretion; however, it did not replace the skeletal survey. If a radionuclide bone scan was used at screening, in addition to the complete skeletal survey, then both methods were used to document disease status. These tests were performed at the same time. A radionuclide bone scan did not replace a complete skeletal survey. If a subject presented with disease progression manifested by symptoms of pain due to bone changes, then disease progression was documented by skeletal survey or other radiographs, depending on the symptoms that the subject experiences. If the diagnosis of disease progression was obvious by radiographic investigations, then no repeat confirmatory X-rays were thought necessary to perform. If changes were equivocal, then a repeat X-ray was performed in 1 to 3 weeks.
Documentation of Extramedullary Plasmacytomas: Sites of known extramedullary plasmacytomas were documented ≤14 days prior to the first dose of the conditioning regimen. Clinical examination or MRI were used to document extramedullary sites of disease. CT scan evaluations were considered an acceptable alternative if there was no contraindication to the use of IV contrast. Positron emission tomography scan or ultrasound tests were not acceptable to document the size of extramedullary plasmacytomas. However, PET/CT fusion scans were optionally used to document extramedullary plasmacytomas if the CT component of the PET/CT fusion scan was of sufficient diagnostic quality. Extramedullary plasmacytomas were assessed for all subjects with a history of plasmacytomas or if clinically indicated at ≤14 days prior to the first dose of the conditioning regimen, by clinical examination or radiologic imaging. Assessment of measurable sites of extramedullary disease were performed, measured, and evaluated locally every 4 weeks (for physical examination) for subjects with a history of plasmacytomas or as clinically indicated during treatment for other subjects until development of confirmed CR or confirmed disease progression. If assessment could only be performed radiologically, then evaluation of extramedullary plasmacytomas was done every 12 weeks. Irradiated or excised lesions were considered not measurable and were monitored only for disease progression. To qualify for VGPR or PR/ minimal response (MR), the sum of products of the perpendicular diameters of the existing extramedullary plasmacytomas must have decreased by over 90% or at least 50%, respectively, and new plasmacytomas must not have developed. To qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. When not all existing extramedullary plasmacytomas were reported, but the sum of products of the perpendicular diameters of the reported plasmacytomas had increased by at least 50%, then the criterion for disease progression was met.

If it was determined that the study treatment interfered with the immunofixation assay, CR was defined as the disappearance of the original M-protein associated with multiple myeloma on immunofixation, and the determination of CR was not affected by unrelated M-proteins secondary to the study treatment.

Study endpoints, as assessed by an independent review committee (IRC), were as follows:
Overall response rate (ORR) was defined as the proportion of subjects who achieved a PR or better according to the IMWG criteria.
VGPR or better response rate (sCR+CR+VGPR) was defined as the proportion of subjects who achieve a VGPR or better response according to the IMWG criteria.
Duration of response (DOR) was calculated among responders (with a PR or better response) from the date of initial documentation of a response (PR or better) to the date of first documented evidence of progressive disease, as defined in the IMWG criteria. Relapse from CR by positive immunofixation or trace amount of M-protein was not considered as disease progression. Disease evaluations continued beyond relapse from CR until disease progression was confirmed.
Time to response (TTR) was defined as the time between date of the initial infusion of cilta-cel and the first efficacy evaluation at which the subject had met all criteria for PR or better.
Progression-free survival (PFS) was defined as the time from the date of the initial infusion of cilta-cel to the date of first documented disease progression, as defined in the IMWG criteria, or death due to any cause, whichever occurred first.
Overall survival (OS) was measured from the date of the initial infusion of cilta-cel to the date of the subject’s death.

For ORR, the response rate and its 95% exact confidence interval (CI) was calculated based on binomial distribution, and the null hypothesis was rejected if the lower bound of the confidence interval exceeded 30%. Analysis of VGPR or better response rate, DOR, PFS, and OS was conducted at the same cutoff as the ORR. The distribution (median and Kaplan-Meier curves) of DOR was provided using Kaplan-Meier estimates. Similar analysis was performed for OS, PFS, and TTR.

### 5.4. Example 4: Evaluation of Safety of Method of Treating Cohort A, Cohort B and Cohort C With Ciltacabtagene Autoleucel

Adverse events were followed, reported and graded according to the National Cancer Institute Common Terminology Criteria for Adverse Events (NCI-CTCAE Version 5.0), with the exception of CRS and CAR-T cell-related neurotoxicity (e.g., ICANS). CRS was evaluated according to the ASTCT consensus grading, summarized in Table 30. At the first sign of CRS (such as fever), subjects were immediately hospitalized for evaluation. Tocilizumab intervention was discretionally used to treat subjects presenting symptoms of fever when other sources of fever had been eliminated. Tocilizumab was discretionally used for early treatment in subjects at high risk of severe CRS (for example, high baseline tumor burden, early fever onset, or persistent fever after 24 hours of symptomatic treatment). Other monoclonal antibodies targeting cytokines (for example, anti-IL1 and/or anti-TNFα) were optionally used, especially for cases of CRS which did not respond to tocilizumab.

CAR-T cell-related neurotoxicity (e.g., ICANS) was graded using the ASTCT consensus grading, summarized in Table 31. Additionally, all individual symptoms of CRS (e.g., fever, hypotension) and ICANS (e.g., depressed level of consciousness, seizures) were captured as individual adverse events and graded by CTCAE criteria. Neurotoxicity that was not temporarily associated with CRS, or any other neurologic adverse events that did not qualify as ICANS, were graded by CTCAE criteria. Any adverse event or serious adverse event not listed in the NCI CTCAE Version 5.0 was graded according to investigator clinical judgment by using the standard grades as follows:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.
Grade 4: Life-threatening consequences; urgent intervention indicated.
Grade 5: Death related to adverse event.

### 5.5. Example 5: Efficacy and Safety of Ciltacabtagene Autoleucel in Lenalidomide-Refractory Patients With Progressive Multiple Myeloma After 1-3 Prior Lines of Therapy (Cohort A)

Treatment options are limited for patients with progressive multiple myeloma (MM) who are refractory to lenalidomide and/or proteasome inhibitors (PI). Patients in Cohort A had progressive MM after 1-3 prior lines of therapy, including a PI and immunomodulatory drug (IMiD), were lenalidomide-refractory, and had no prior exposure to BCMA-targeting agents. A single cilta-cel infusion (target dose 0.75×10⁶ CAR+ viable T cells/kg) was given 5-7 days after start of lymphodepletion (daily cyclophosphamide [300 mg/m²] and fludarabine [30 mg/m²] for 3 days). Bridging therapy was allowed after apheresis. The primary endpoint was minimal residual disease (MRD) negativity at a sensitivity level of 10⁻⁵. Secondary endpoints were overall response rate (ORR = Rate of partial response or better), duration of response, time and duration of MRD negativity, and incidence and severity of adverse events. MRD was assessed by next-generation sequencing, response was assessed per IMWG criteria, and adverse events were graded using Version 5.0 of the National Cancer Institute Common Terminology Criteria for Adverse Events (CTCAEv5.0). Cytokine release syndrome [CRS] and immune effector cell-associated neurotoxicity [ICANS] were graded according to the American Society of Transplantation and Cellular Therapy (ASTCT) consensus grading system.

As of the Apr. 15, 2021 data cutoff (median follow-up 9.7 months; range 3.3-13.4 months), 20 patients (65% male; median age 60 years [range 38-75 years]) received cilta-cel; 1 patient was treated in an outpatient setting. Patients received a median of 2 prior lines of therapy (range 1-3); 60% of patients received 1-2 prior lines of therapy and 40% received 3 prior lines of therapy. All patients were exposed to a PI, IMiD, and dexamethasone, 95% to alkylating agents, and 65% to daratumumab. In all, 95% of patients were refractory to the last line of therapy; 40% were triple-class refractory.

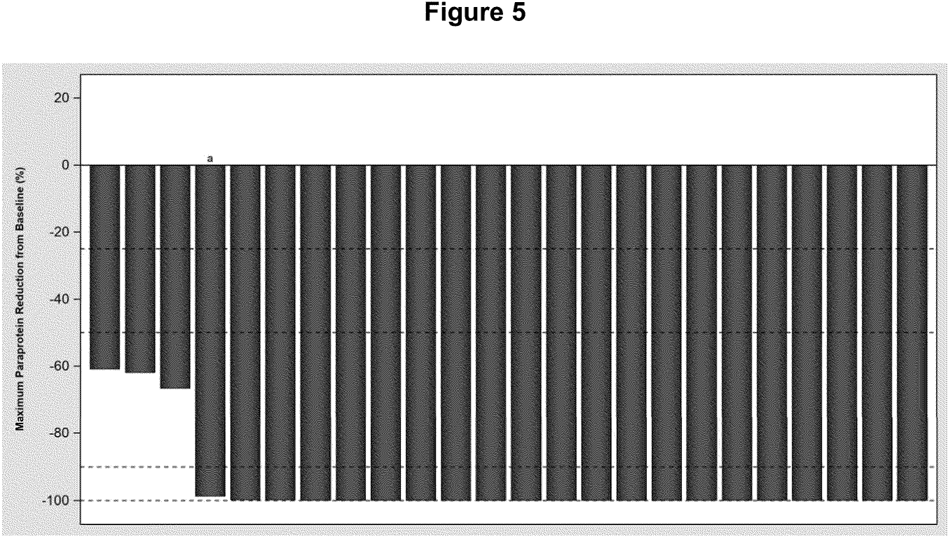

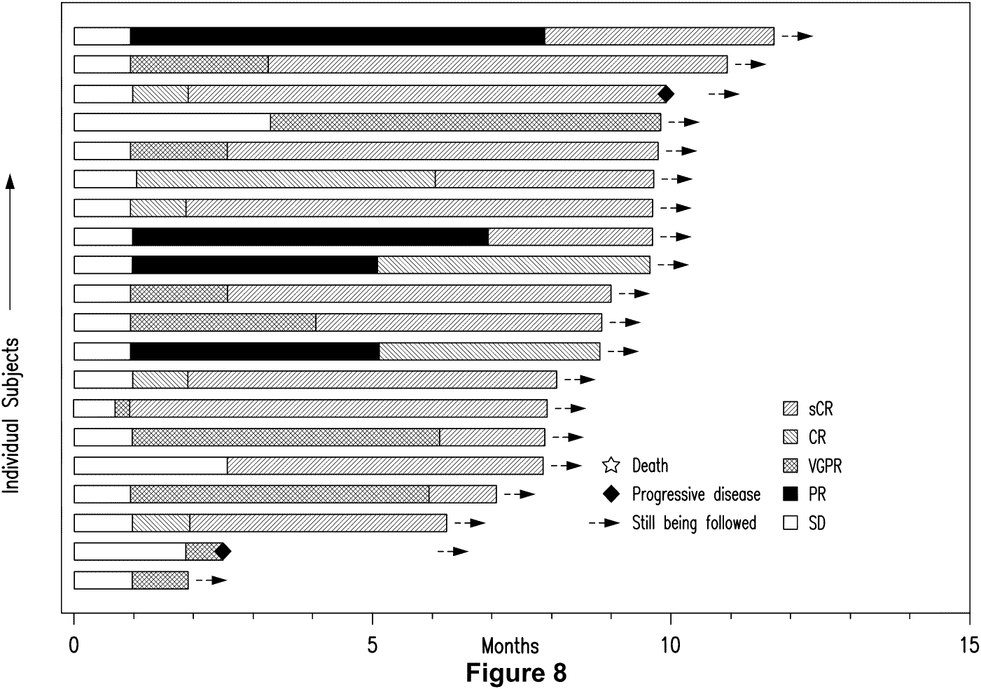

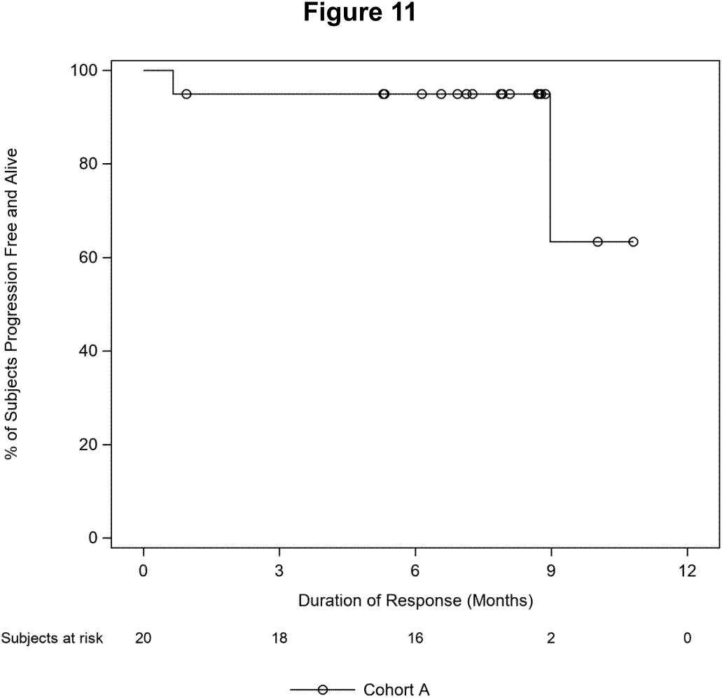

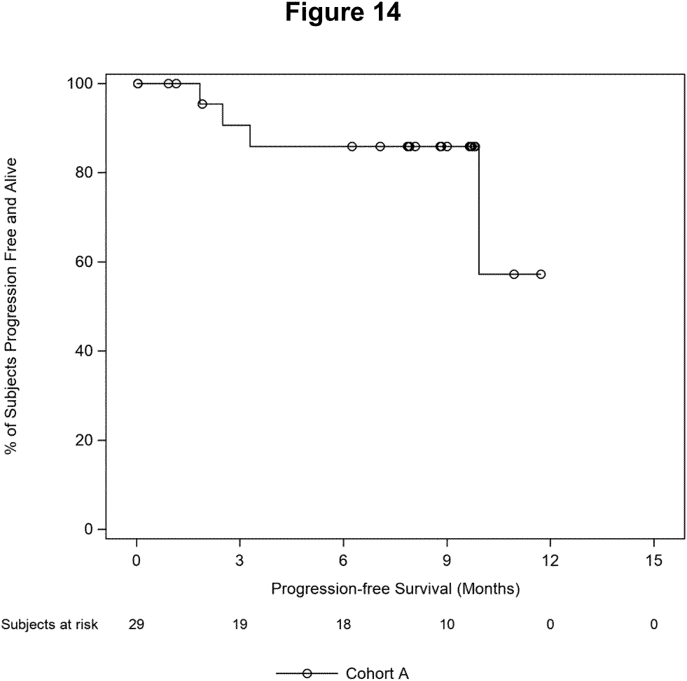

The following efficacy parameters were assessed:
Tumor burden: Tumor burden at cilta-cel infusion was based on the last non-missing value on or prior to the date of cilta-cel infusion and after apheresis. For subjects with measurable disease by serum M-protein and urine M-protein, the worst case scenario of change in tumor burden is presented, i.e., the largest increase in tumor burden for subjects observed with increased tumor burden and the smallest decrease in tumor burden for subjects observed with decreased tumor burden. FIG. 5 shows the reduction in disease burden (representing the type of measurable disease, i.e., serum M-protein, urine M-protein, or the difference between involved and uninvolved free light chain (dFLC)) in Cohort A responders in the All Treated Analysis Set. “a” denotes Bence-Jones proteinuria at baseline with a transient response during bridging therapy; output represents dFLC value. Table 3 shows the change in tumor burden from screening to cilta-cel infusion in Cohort A responders in the All Treated Analysis Set.
Minimal residual disease (MRD): Table 6 shows the listing of MRD data for Cohort A responders in the All Treated Analysis Set. The study day is in reference to the retreatment of cilta-cel (Day 1). Table 9 shows the summary of the overall MRD negativity rate in the bone marrow of Cohort A responders in the All Treated Analysis Set. Table 12 shows the summary of the overall MRD negativity rate in the bone marrow of Cohort A responders in the All Treated Analysis Set with evaluable sample at a sensitivity level of 10⁻⁵. Evaluable samples are those that pass calibration and quality control and include sufficient cells for evaluation at the respective testing threshold. Of the 13 patients with MRD-evaluable samples at 10⁻⁵ at data cutoff, 12 (92.3% [96% CI 64%-100%]) were MRD-negative.
Responses: Response were assessed based on International Myeloma Working Group (IMWG) consensus criteria (2016). Percentages were calculated with the number of subjects in the All Treated Analysis Set as the denominator. For the assessment of MRD-negative CR/sCR, only MRD assessments (at the 10⁻⁵ testing threshold) within 3 months of achieving CR/sCR until death, progression or subsequent therapy (exclusive of cilta-cel) were considered. Table 15 shows the listing of disease response assessment for Cohort A responders in the All Treated Analysis Set based on the initial cilta-cel infusion date (Day 1) as the reference. FIG. 8 shows the response and duration of response in Cohort A responders in the All Treated Analysis Set. Table 18 shows the overall best response for Cohort A responders in the All Treated Analysis Set. The ORR was 95% (95% CI 75-100); the ≥CR rate was 85% (95% CI 62-97) and the ≥VGPR rate was 95% (95% CI 75-100). Table 21 shows the times to first response, best response, and complete response or better for Cohort A responders in the All Treated Analysis Set. Median time to first response was 1.0 month (range 0.7-3.3); median time to best response was 3.3 months (range 0.9-7.9). Table 24 shows the assessment of the duration of response for Cohort A responders in the All Treated Analysis Set. FIG. 11 shows a Kaplan-Meier plot for the assessment of the duration of response in Cohort A responders in the All Treated Analysis Set. Median duration of response was not reached.
Progression-free survival (PFS): FIG. 14 shows a Kaplan-Meier plot for the assessment of progression-free survival in Cohort A responders in the All Treated Analysis Set. Table 27 shows the assessment of the progression-free survival in Cohort A responders in the All Treated Analysis Set.

Safety parameters were also assessed. Hematologic adverse events in ≥20% of patients were neutropenia (95%; grade ¾ 95%), thrombocytopenia (80%; grade ¾ 35%), anemia (75%; grade ¾ 45%), lymphopenia (65%; grade ¾ 60%) and leukopenia (55%; grade ¾ 55%). CRS occurred in 95% of patients (grade ¾: 10%). Median time to CRS onset was 7 days (range 5-9), with a median duration of 4.0 days (range 2-11). CRS resolved within 7 days in 90% of patients. CAR T-cell neurotoxicity occurred in 4 (20%) patients (3 grade ½). Three patients (15%) had ICANS (all grade ½); median time to onset was 8 days (range 7-10 days) and median duration was 3 days (range 1-3 days). One patient had grade 2 facial paralysis; time to onset was 29 days with a duration of 51 days. No movement or neurocognitive adverse events were observed. One death occurred due to COVID-19 (assessed as treatment-related by the investigator). The safety profile was manageable in the patient treated in an outpatient setting.

In conclusion, at a median follow-up of 9.7 months, a single cilta-cel infusion led to early and deep responses in patients with multiple myeloma who had 1-3 prior lines of therapy and were lenalidomide-refractory. Responses deepened over time, with 93% of patients achieving MRD 10⁻⁵ negativity at data cutoff. The safety profile was manageable with CRS mostly grade ½ and no movement or neurocognitive adverse events observed, highlighting the success of monitoring and patient management strategies implemented across phase ⅔ studies in the clinical study program disclosed herein. The early and deep responses in this patient population were unexpectedly superior to expected outcomes with the standard of care line of therapy.

### 5.6. Example 6: Efficacy and Safety of Ciltacabtagene Autoleucel in Patients With Early Relapse After Front-Line Therapy (Cohort B)

Standard frontline therapies for patients with multiple myeloma (MM) include proteasome inhibitors (PIs) and immunomodulatory drugs (IMiDs). However, some patients experience early relapses defined as disease progression ≤12 months after autologous stem cell transplantation (ASCT) or start of frontline therapies. These patients have been shown to have short overall survival rates and remain at high risk for poor outcomes. Here, we present results from Cohort B, i.e., patients with an early relapse after 1 prior line of therapy, including a PI and IMiD.

Eligible patients had documented multiple myeloma per International Myeloma Working Group (IMWG) criteria, received 1 prior line of therapy that included a PI and IMiD, disease progression per IMWG criteria either ≤12 months after ASCT or ≤12 months after start of anti-myeloma therapy for patients who did not undergo ASCT, and no prior exposure to CAR-T or BCMA-targeting treatments. A single cilta-cel infusion (target dose 0.75×10⁶ CAR+ viable T cells/kg) was given 5-7 days after start of lymphodepletion (300 mg/m² cyclophosphamide and 30 mg/m² fludarabine daily for 3 days). Bridging therapy was allowed after apheresis when clinically indicated. The primary objective was minimal residual disease (MRD) negativity at 10⁻⁵, as assessed by next generation sequencing. Secondary outcomes were overall response rate (ORR), duration of response, time to response, and incidence and severity of adverse events. Adverse events were graded using CTCAEv5.0 (CRS and ICANS by ASTCT).

As of the Apr. 15, 2021 data cutoff (median follow up 4.7 months [range, 0.6-13.5 months]), 18 patients (median age 57.0 years [range, 44-67 years]; 78% male) received cilta-cel; 72% of patients had baseline bone marrow plasma cells ≤30%. In this cohort, 89% of patients received bridging therapy; 38% of patients had ≥25% increase in tumor burden despite bridging therapy.

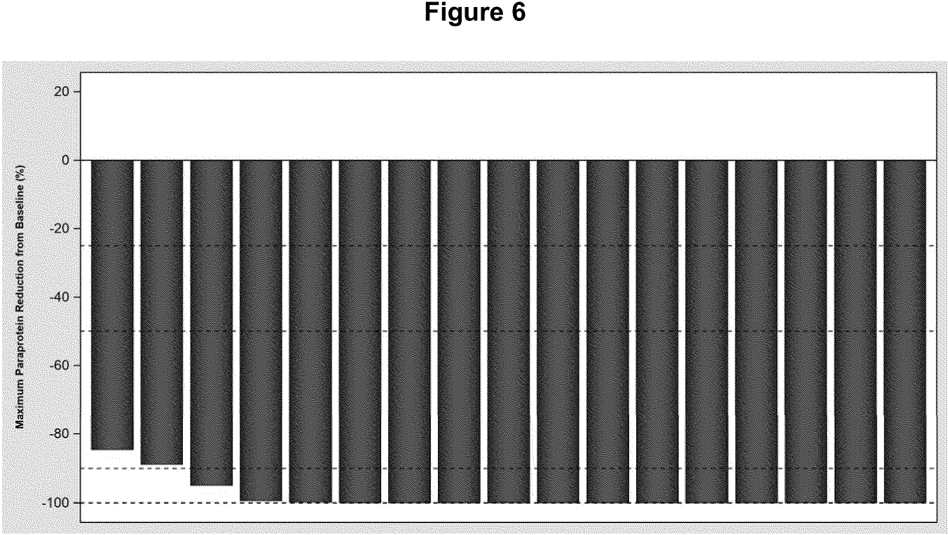

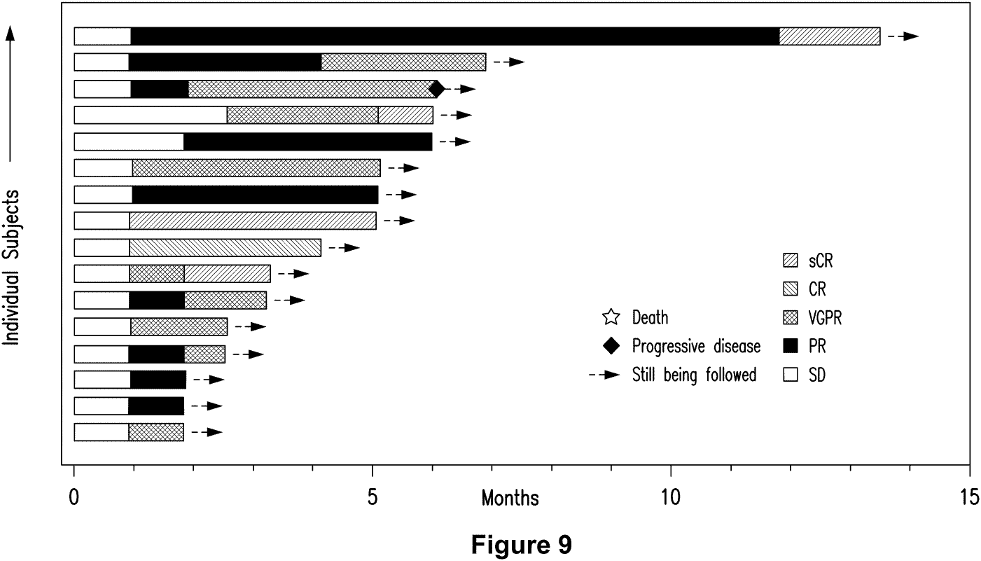

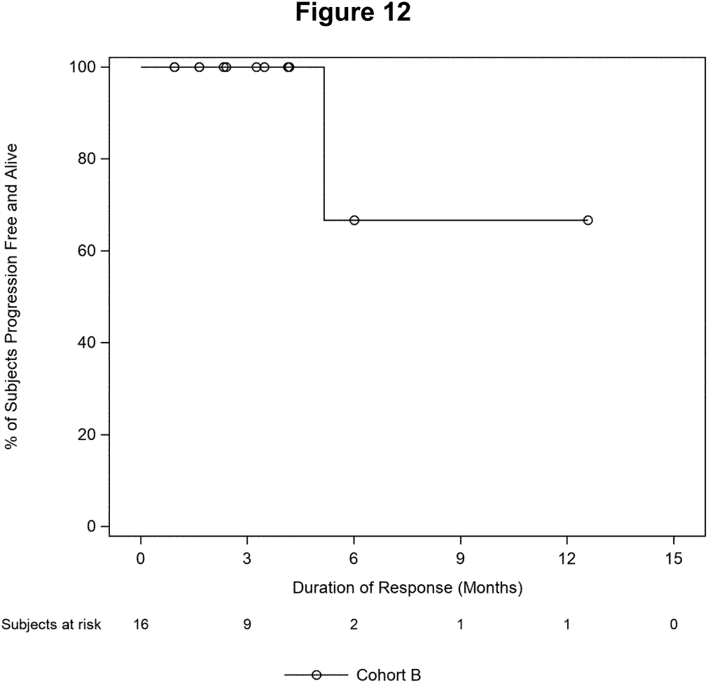

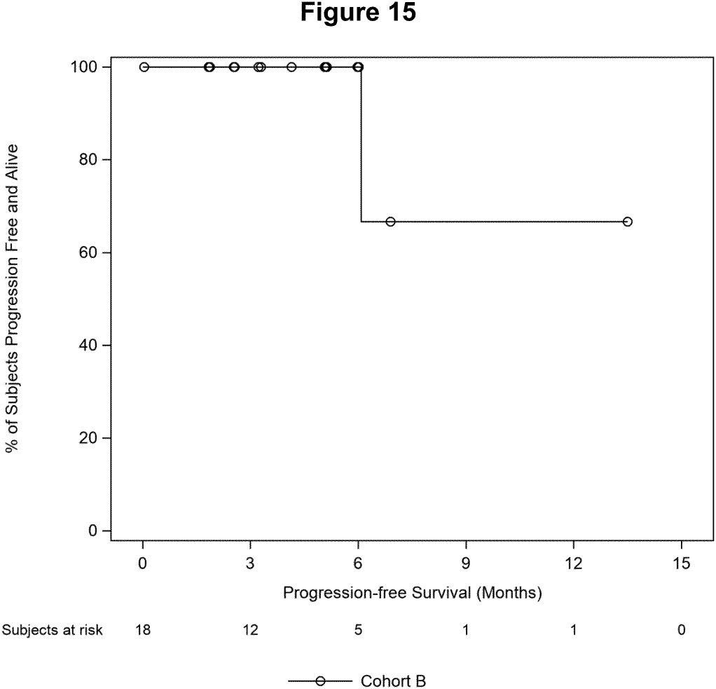

The following efficacy parameters were assessed:
Tumor burden: Tumor burden at cilta-cel infusion was based on the last non-missing value on or prior to the date of cilta-cel infusion and after apheresis. For subjects with measurable disease by serum M-protein and urine M-protein, the worst case scenario of change in tumor burden is presented, i.e., the largest increase in tumor burden for subjects observed with increased tumor burden and the smallest decrease in tumor burden for subjects observed with decreased tumor burden. FIG. 6 shows the reduction in disease burden (representing the type of measurable disease, i.e., serum M-protein, urine M-protein, or the difference between involved and uninvolved free light chain (dFLC)) in Cohort B responders in the All Treated Analysis Set. “a” denotes Bence-Jones proteinuria at baseline with a transient response during bridging therapy; output represents dFLC value. Table 4 shows the change in tumor burden from screening to cilta-cel infusion in Cohort B responders in the All Treated Analysis Set.
Minimal residual disease (MRD): Table 7 shows the listing of MRD data for Cohort B responders in the All Treated Analysis Set. The study day is in reference to the retreatment of cilta-cel (Day 1). Table 10 shows the summary of the overall MRD negativity rate in the bone marrow of Cohort B responders in the All Treated Analysis Set. Table 13 shows the summary of the overall MRD negativity rate in the bone marrow of Cohort B responders in the All Treated Analysis Set with evaluable sample at a sensitivity level of 10⁻⁵. Evaluable samples are those that pass calibration and quality control and include sufficient cells for evaluation at the respective testing threshold.
Responses: Response were assessed based on International Myeloma Working Group (IMWG) consensus criteria (2016). Percentages were calculated with the number of subjects in the All Treated Analysis Set as the denominator. For the assessment of MRD-negative CR/sCR, only MRD assessments (at the 10⁻⁵ testing threshold) within 3 months of achieving CR/sCR until death, progression or subsequent therapy (exclusive of cilta-cel) were considered. Table 16 shows the listing of disease response assessment for Cohort B responders in the All Treated Analysis Set based on the initial cilta-cel infusion date (Day 1) as the reference. FIG. 9 shows the response and duration of response in Cohort B responders in the All Treated Analysis Set. Table 19 shows the overall best response for Cohort B responders in the All Treated Analysis Set. ORR was 88.9% (95% CI: 65.3-98.6%). 27.8% of patients (95% CI: 9.7-53.5%) achieved complete response or better (≥CR), and 66.7% (95% CI:41.0-86.7%) achieved very good partial response or better. Table 22 shows the times to first response, best response, and complete response or better for Cohort B responders in the All Treated Analysis Set. Median time to first response was 0.9 months (range, 0.9-2.6 months), median time to best response was 1.4 months (range, 0.9-11.8 months), and median time to ≥CR was 1.8 months (range, 0.9-11.6 months). Table 25 shows the assessment of the duration of response for Cohort B responders in the All Treated Analysis Set. FIG. 12 shows a Kaplan-Meier plot for the assessment of the duration of response in Cohort B responders in the All Treated Analysis Set.
Progression-free survival (PFS): FIG. 15 shows a Kaplan-Meier plot for the assessment of progression-free survival in Cohort B responders in the All Treated Analysis Set. Table 28 shows the assessment of the progression-free survival in Cohort B responders in the All Treated Analysis Set.

Safety parameters were also assessed. Hematologic treatment-emergent adverse events in ≥20% of patients were neutropenia (88.9%; grade ¾: 83.3%), thrombocytopenia (61.1%; grade ¾: 27.8%), anemia (50%; grade ¾: 44.4%), leukopenia (27.8%; all grade ¾), and lymphopenia (22.2%; all grade ¾). CRS was observed in 15 (83.3%) patients (1 grade ¾). Median time to CRS onset was 8 days (range, 5-11 days), and median CRS duration was 4 days (range, 1-7 says). CRS resolved in all patients within 7 days. Grade 1 ICANS was observed in 1 (5.6%) patient with a median time of onset of 11 days, and median duration of 4 days. One patient had grade 4 movement and neurocognitive treatment-emergent adverse events and presented with bradykinesia and motor dysfunction; this patient was refractory to multiple therapies and, despite bridging therapy, showed disease progression between screening and baseline. The patient was treated with high-dose methylprednisolone, plasmapheresis, and IV immunoglobulin, and is currently reported to be in stable condition. To minimize the risk of movement and neurocognitive treatment-emergent adverse events, several patient management strategies were implemented including effective bridging therapy to reduce baseline tumor burden, early aggressive treatment of CRS and ICANS, handwriting assessment tools for early detection of neurotoxicity symptoms, and extended monitoring and reporting time of CAR-T cell neurotoxicities up to 1 year after cilta-cel infusion. No deaths were reported in Cohort B.

In conclusion, a single cilta-cel infusion led to early and deep responses with a manageable safety profile in patients who relapsed early after front-line therapy, including a PI and IMiD. Neurotoxicities associated with cilta-cel can be detected with vigilant monitoring, and when identified early, were generally manageable with successful patient management strategies. The early and deep responses in this patient population were unexpectedly superior to expected outcomes with the standard of care line of therapy.

### 5.7. Example 7: Efficacy and Safety of Ciltacabtagene Autoleucel in Patients With Progressive Multiple Myeloma After Exposure to Other BCMA-Targeting Agents (Cohort C)

Anti-B-cell maturation antigen (BCMA) therapies are promising novel treatments for multiple myeloma (MM). However, patients still relapse after these therapies, and further treatment options are limited. In the multicohort, open-label phase 2 study described herein, we evaluated cilta-cel safety and efficacy in various clinical settings for pts with multiple myeloma, and explored treatment sequencing for BCMA-directed agents in multiple. Here, we present initial results from cohort C in patients with prior exposure to other anti-BCMA agents.

Eligible patients were ≥18 y old with progressive MM after treatment with a proteasome inhibitor, immunomodulatory drug, anti-CD38 antibody, and BCMA-targeting agent (not cellular therapy). A single cilta-cel infusion (target dose: 0.75×10⁶ CAR+ viable T cells/kg) was given 5-7 days after start of lymphodepletion (daily cyclophosphamide [300 mg/m²] and fludarabine [30 mg/m²] for 3 days). The primary objective was minimal residual disease (MRD) negativity (10⁻⁵; assessed by next-generation sequencing). Secondary outcomes were efficacy (per IMWG criteria) and adverse events per CTCAE (CRS and ICANS by ASTCT).

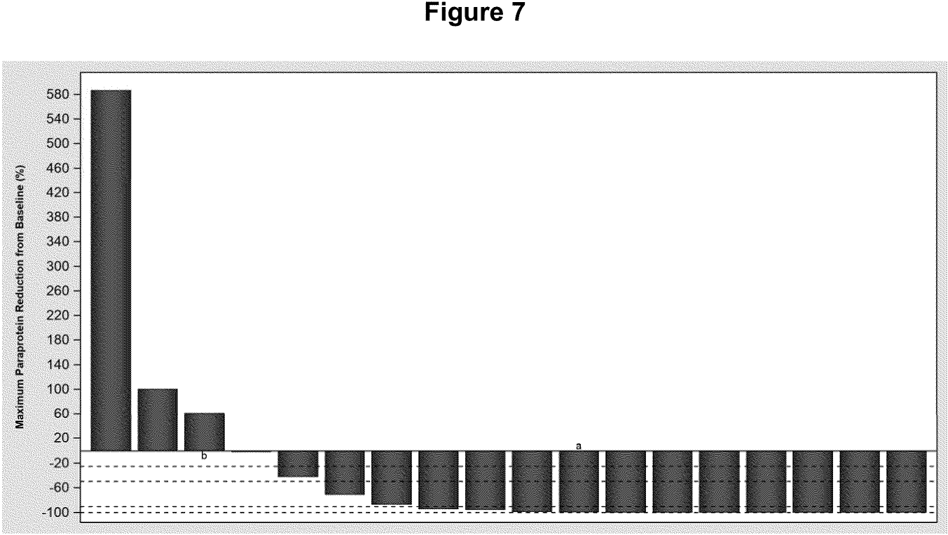

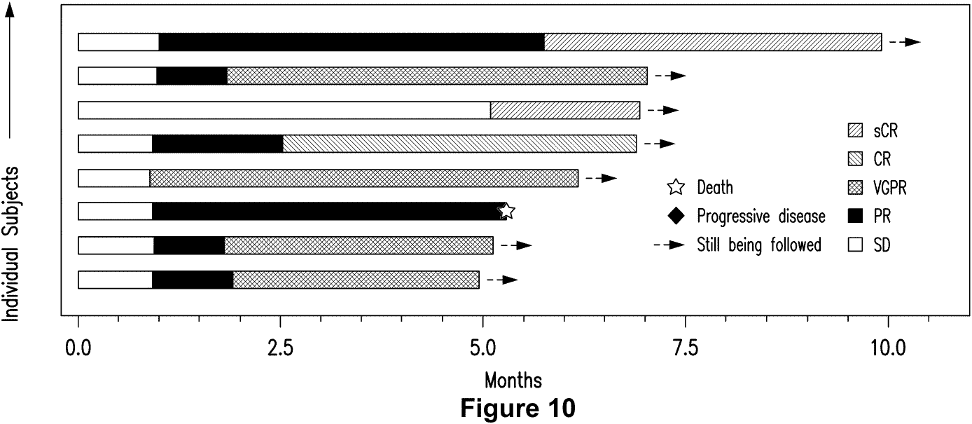

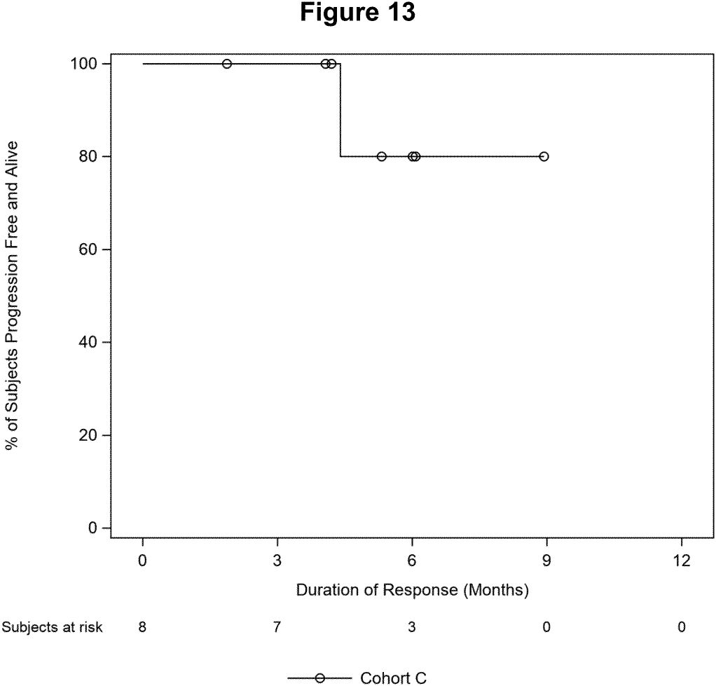

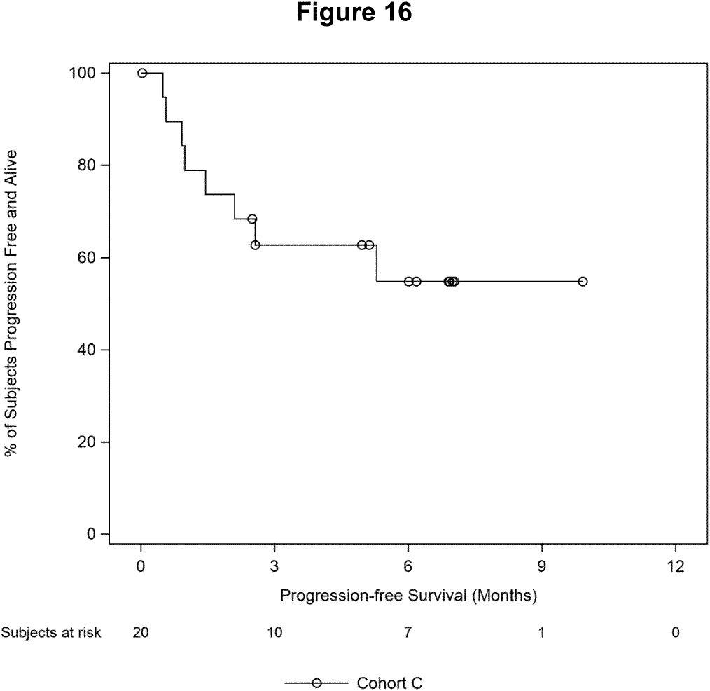

As of the Apr. 15, 2021 data cutoff (median follow-up 6.2 months; range 0.4-10.5 months), 20 patients (60% male; median age 63 years [range 44-81 years]) in cohort C received cilta-cel-patients received a median of 8 prior lines of therapy (range 4-13). All had prior exposure to an anti-BCMA agent: 40% received an anti-BCMA bispecific antibody (Ab), and 65% received an anti-BCMA antibody-drug conjugate (ADC). 90% of patients were refractory to the last line of therapy; 90% were triple-refractory, and 80% were refractory to an anti-BCMA agent. All patients had soluble BCMA expression at baseline (BL); 53% of patients had BL bone marrow plasma cells ≤30%. Most patients (90%) received bridging therapy; 35% of patients had ≥25% increase in tumor burden during the period of bridging therapy.
Tumor burden: Tumor burden at cilta-cel infusion was based on the last non-missing value on or prior to the date of cilta-cel infusion and after apheresis. For subjects with measurable disease by serum M-protein and urine M-protein, the worst case scenario of change in tumor burden is presented, i.e., the largest increase in tumor burden for subjects observed with increased tumor burden and the smallest decrease in tumor burden for subjects observed with decreased tumor burden. FIG. 7 shows the reduction in disease burden (representing the type of measurable disease, i.e., serum M-protein, urine M-protein, or the difference between involved and uninvolved free light chain (dFLC)) in Cohort C responders in the All Treated Analysis Set. “a” denotes Bence-Jones proteinuria at baseline with a transient response during bridging therapy; output represents dFLC value. Table 5 shows the change in tumor burden from screening to cilta-cel infusion in Cohort C responders in the All Treated Analysis Set.
Minimal residual disease (MRD): Table 8 shows the listing of MRD data for Cohort C responders in the All Treated Analysis Set. The study day is in reference to the retreatment of cilta-cel (Day 1). Table 11 shows the summary of the overall MRD negativity rate in the bone marrow of Cohort C responders in the All Treated Analysis Set. Table 14 shows the summary of the overall MRD negativity rate in the bone marrow of Cohort C responders in the All Treated Analysis Set with evaluable sample at a sensitivity level of 10⁻⁵. Evaluable samples are those that pass calibration and quality control and include sufficient cells for evaluation at the respective testing threshold. Of the 6 patients with MRD-evaluable samples at 10⁻⁵ at data cutoff, 4 (67% [96% CI 22-96%]) were MRD-negative.
Responses: Response were assessed based on International Myeloma Working Group (IMWG) consensus criteria (2016). Percentages were calculated with the number of subjects in the All Treated Analysis Set as the denominator. For the assessment of MRD-negative CR/sCR, only MRD assessments (at the 10⁻⁵ testing threshold) within 3 months of achieving CR/sCR until death, progression or subsequent therapy (exclusive of cilta-cel) were considered. Table 17 shows the listing of disease response assessment for Cohort C responders in the All Treated Analysis Set based on the initial cilta-cel infusion date (Day 1) as the reference. FIG. 10 shows the response and duration of response in Cohort C responders in the All Treated Analysis Set. Table 20 shows the overall best response for Cohort C responders in the All Treated Analysis Set. ORR was 40%, with 35% achieving ≥VGPR. Table 23 shows the times to first response, best response, and complete response or better for Cohort C responders in the All Treated Analysis Set. Median time to first response was 0.9 months (range 0.9-5.1 months). Over half the patients (57%) with prior bispecific Ab and 38% with prior ADC therapy achieved a response with cilta-cel (i.e., they were responders). Table 26 shows the assessment of the duration of response for Cohort C responders in the All Treated Analysis Set. FIG. 13 shows a Kaplan-Meier plot for the assessment of the duration of response in Cohort C responders in the All Treated Analysis Set.
Progression-free survival (PFS): FIG. 16 shows a Kaplan-Meier plot for the assessment of progression-free survival in Cohort C responders in the All Treated Analysis Set. Table 29 shows the assessment of the progression-free survival in Cohort C responders in the All Treated Analysis Set.

Table 32 shows the response to cilta-cel in Cohort C depending on the parameters of prior anti-BCMA exposure. Potential predictors of response included shorter median duration of exposure to prior anti-BCMA therapy, and longer intervals between prior exposure and apheresis and prior exposure and infusion in responders vs non-responders (Table). Serum BCMA expression at baseline appeared to predict response to subsequent treatment in patients with prior anti-BCMA therapy; cilta-cel responders had higher levels of baseline serum BCMA (>25 ng/mL) vs non-responders (<25 ng/mL). CAR T-cell expansion was higher in responders vs non-responders, with more robust expansion in patients with prior bispecific Ab vs ADC therapy.

Safety profile was generally manageable. Grade ¾ hematologic adverse events were neutropenia (80%), thrombocytopenia (65%), anemia (55%), leukopenia (50%), and lymphopenia (30%). CRS occurred in 12 (60%) patients; all were grade ½. Median time to CRS onset was 7.5 days (range 2-10 days); median duration was 7 days (range 2-11 days). CAR T-cell neurotoxicity occurred in 4 patients (20%; 2 grade ¾); 3 patients had ICANS (2 grade ¾) with a median time to onset of 11 days (range 10-13 days) and a median duration of 9 days (range 4-14 days). There were no movement and neurocognitive treatment-emergent adverse events. Four patients died during the study: one each from progressive disease, acute respiratory failure (Covid-19), subarachnoidal hemorrhage, and treatment-related C. difficile colitis.

In conclusion, cilta-cel yielded early and effective responses in patients who had prior exposure to other BCMA-targeting therapies, including robust responses in those who received prior bispecific Ab and ADC therapies. The early and effective responses in this patient population were unexpectedly superior to expected outcomes with the standard of care line of therapy.

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

### TABLES

TABLE 1

Pre-infusion Medications

| | | |
|---|---|---|
| Medication | Dose | Administration |
| Antihistamine | diphenhydramine (50 mg) or equivalent | Oral - administer 1 hour (±15 minutes) prior to cilta-cel infusion Or IV- start infusion 30 minutes (±15 minutes) prior to cilta-cel infusion |
| Antipyretic | acetaminophen (650 mg to 1,000 mg) or equivalent | Oral or IV - administer 30 minutes (±15 minutes) prior to cilta-cel infusion |

TABLE 2

Criteria for Response to Multiple Myeloma Treatment

| | |
|---|---|
| Response | Response Criteria |
| Stringent complete response (sCR) | • CR as defined below, plus • Normal FLC ratio, and • Absence of clonal plasma cells (PCs) by immunohistochemistry or 2- to 4-color flow cytometry |
| Complete response (CR)^{a} | • Negative immunofixation of serum and urine, and • Disappearance of any soft tissue plasmacytomas, and • <5% PCs in bone marrow • No evidence of initial monoclonal protein isotype(s) on immunofixation of the serum and urine.^{b} |
| Very good partial response (VGPR)^{a} | • Serum and urine M-component detectable by immunofixation but not on electrophoresis, or • ≥90% reduction in serum M-component plus urine M-component <100 mg/24 hours |
| Partial response (PR) | • ≥50% reduction of serum M-protein and reduction in 24-hour urinary M-protein by ≥90% or to < 200 mg/24 hours • If serum and urine M-protein were not measurable, a decrease ≥50% in the difference between involved and uninvolved FLC levels was required in place of the M-protein criteria • If serum and urine M-protein were not measurable, and serum FLC assay was also not measurable, ≥50% reduction in bone marrow PCs was required in place of M-protein, provided baseline percentage had been ≥30% • In addition to the above criteria, if present at baseline, ≥50% reduction in the size of soft tissue plasmacytomas was also required. |
| Minimal response (MR) | • ≥25% but ≤49% reduction of serum M-protein and reduction in 24-hour urine M-protein by 50% to 89% • In addition to the above criteria, if present at baseline, ≥50% reduction in the size of soft tissue plasmacytomas was also required. |
| Stable disease | • Not meeting criteria for sCR, CR, VGPR, PR, MR, or progressive disease |
| Progressive disease^{c} | Any one or more of the following criteria: • Increase of 25% from lowest response value in any of the following: - Serum M-component (absolute increase must be ≥0.5 g/dL), and/or - Urine M-component (absolute increase must be ≥200 mg/24 hours), and/or - Only in subjects without measurable serum and urine M-protein levels: the difference between involved and uninvolved FLC levels (absolute increase must be > 10 mg/dL) - Only in subjects without measurable serum and urine M-protein levels and without measurable disease by FLC levels, bone marrow PC percentage (absolute increase must be ≥10%). • Appearance of a new lesion(s), ≥50% increase from nadir in sum of the products of the maximal perpendicular diameters of measured lesions of > 1 lesion, or ≥50% increase in the longest diameter of a previous lesion >1 cm in short axis • Definite development of new bone lesions or definite increase in the size of existing bone lesions • ≥50% increase in circulating plasma cells (minimum of 200 cells per µL) if this was the only measure of disease |

^{a} Clarifications to the criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a ≥90% decrease in the difference between involved and uninvolved FLC levels. For patients achieving very good partial response by other criteria, a soft tissue plasmacytoma must decrease by more than 90% in the sum of the maximal perpendicular diameter (SPD) compared with baseline.
^{b.} In some cases it is possible that the original M protein light-chain isotype is still detected on immunofixation but the accompanying heavy-chain component has disappeared; this would not be considered as a CR even though the heavy-chain component is not detectable, since it is possible that the clone evolved to one that secreted only light chains. Thus, if a patient has IgA lambda myeloma, then to qualify as CR there should be no IgA detectable on serum or urine immunofixation; if free lambda is detected without IgA, then it must be accompanied by a different heavy chain isotype (IgG, IgM, etc.).
^{c.} Clarifications to the criteria for coding progressive disease: bone marrow criteria for progressive disease are to be used only in subjects without measurable disease by M-protein and by FLC levels; “25% increase” refers to M-protein, and FLC, and does not refer to bone lesions, or soft tissue plasmacytomas and the “lowest response value” does not need to be a confirmed value.
Notes: All response categories (CR, sCR, VGPR, PR, MR, and progressive disease) require 2 consecutive assessments made at any time before the institution of any new therapy; CR, sCR, VGPR, PR, MR, and stable disease categories also require no known evidence of progressive or new bone lesions if radiographic studies were performed. VGPR and CR categories require serum and urine studies regardless of whether disease at baseline was measurable on serum, urine, both, or neither.
Radiographic studies are not required to satisfy these response requirements. Bone marrow assessments need not be confirmed. For progressive disease, serum M-component increases of ≥1 g/dL are sufficient to define relapse if lowest M-component is ≥5 g/dL.
Source: Adapted from Durie (2015) and Rajkumar (2011), Kumar (2016)

TABLE 3

Change in Tumor Burden from Screening to Cilta-cel Infusion; Cohort A All Treated Analysis Set

| | | Bridging Therapy | | | |
|---|---|---|---|---|---|
| | Total | | Yes | | No |
| | | Novel Therapy | | | |
| | | Total | Yes | No | |
| Analysis set: all treated | 29 | | | | |

Change in tumor burden from screening to Cilta-cel Infusion

| | | | | | |
|---|---|---|---|---|---|
| N | 29 | 24 | 8 | 16 | 5 |
| Increased | 15 | 13 | 2 | 11 | 2 |
| | (51.7%) | (54.2%) | (25.0%) | (68.8%) | (40.0%) |
| Increase >= 25% | 13 | 11 | 2 | 9 | 2 |
| | (44.8%) | (45.8%) | (25.0%) | (56.3%) | (40.0%) |
| Did not change | 0 | 0 | 0 | 0 | 0 |
| Decreased | 9 | 8 | 4 | 4 | 1 |
| | (31.0%) | (33.3%) | (50.0%) | (25.0%) | (20.0%) |
| Decrease > 50% | 3 | 3 | 1 | 2 | |
| | (10.3%) | (12.5%) | (12.5%) | (12.5%) | 0 |
| Not evaluable | 5 | 3 | 2 | | 2 |
| | (17.2%) | (12.5%) | (25.0%) | 1 (6.3%) | (40.0%) |

TABLE 4

Change in Tumor Burden from Screening to Cilta-cel Infusion; Cohort B All Treated Analysis Set

| | | Bridging Therapy | | | |
|---|---|---|---|---|---|
| | Total | Yes | | | No |
| | | Novel Therapy | | | |
| | | Total | Yes | No | |
| Analysis set: all treated | 18 | | | | |
| Change in tumor burden from screening to Cilta-cel Infusion | | | | | |
| N | 18 | 16 | 10 | 6 | 2 |
| Increased | 10 | 8 | 5 | 3 | 2 |
| | (55.6%) | (50.0%) | (50.0%) | (50.0%) | (100.0%) |
| Increase >= 25% | 6 | 6 | 5 | 1 | |
| | (33.3%) | (37.5%) | (50.0%) | (16.7%) | 0 |
| Did not change | 0 | 0 | 0 | 0 | 0 |

TABLE 4

Change in Tumor Burden from Screening to Cilta-cel Infusion; Cohort B All Treated Analysis Set

| | | |
|---|---|---|
| | Bridging Therapy | |
| Total | Yes | No |

| | | | | | |
|---|---|---|---|---|---|
| | | | Novel Therapy | | |
| | | Total | Yes | No | |
| Decreased | 5 | 5 | 4 | 1 | |
| | (27.8%) | (31.3%) | (40.0%) | (16.7%) | 0 |
| Decrease > 50% | 4 | 4 | 4 | | |
| | (22.2%) | (25.0%) | (40.0%) | 0 | 0 |
| Not evaluable | | 3 | 1 | 2 | |
| | | (18.8%) | (10.0%) | (33.3%) | |
| | 3 (16.7%) | | | | 0 |

TABLE 5

Change in Tumor Burden from Screening to Cilta-cel Infusion; Cohort C All Treated Analysis Set

| | | Bridging Therapy | | | |
|---|---|---|---|---|---|
| | Total | | Yes | | No |
| | | | Novel Therapy | | |
| | | Total | Yes | No | |
| Analysis set: all treated | 20 | | | | |
| Change in tumor burden from screening to Cilta-cel Infusion | | | | | |
| N | 20 | 18 | 7 | 11 | 2 |
| Increased | 9 | 8 | 3 | 5 | 1 |
| | (45.0%) | (44.4%) | (42.9%) | (45.5%) | (50.0%) |
| Increase >= 25% | 7 | 6 | 3 | 3 | 1 |
| | (35.0%) | (33.3%) | (42.9%) | (27.3%) | (50.0%) |
| Did not change | | | 1 | | |
| | 1 (5.0%) | 1 (5.6%) | (14.3%) | 0 | 0 |
| Decreased | 6 | 6 | 2 | 4 | |
| | (30.0%) | (33.3%) | (28.6%) | (36.4%) | 0 |
| Decrease > 50% | 4 | 4 | 1 | 3 | |
| | (20.0%) | (22.2%) | (14.3%) | (27.3%) | 0 |
| Not evaluable | 4 | 3 | 1 | 2 | 1 |
| | (20.0%) | (16.7%) | (14.3%) | (18.2%) | (50.0%) |

TABLE 6

Listing of MRD Data; Cohort A All Treated Analysis Set

| | | | |
|---|---|---|---|
| Date of MRD Assessment (Study Day) | MRD Status at 10⁻⁴ Level | MRD Status at 10⁻⁵ Level | MRD Status at 10⁻⁶ Level |
| 23JUN2020 (58) | Negative | Negative | Negative |
| 27OCT2020 (184) | Negative | Negative | Indeterminate |
| 04FEB2021 (186) | Negative | Negative | Negative |
| 29SEP2020 (58) | Negative | Negative | Negative |
| 14OCT2020 (57) | Positive | Positive | Positive |
| 17FEB2021 (183) | Positive | Positive | Positive |
| 25NOV2020 (99) | Positive | Positive | Positive |
| 03NOV2020 (56) | Negative | Negative | Negative |
| 08MAR2021 (181) | Negative | Negative | Indeterminate |
| 02DEC2020 (59) | Negative | Negative | Negative |
| 12APR2021 (190) | Negative | Negative | Negative |
| 01DEC2020 (184) | Negative | Negative | Negative |
| 08FEB2021 (190) | Negative | Negative | Negative |
| 29SEP2020 (58) | Negative | Negative | Negative |
| 05APR2021 (357) | Negative | Indeterminate | Indeterminate |
| 09JUN2020 (57) | Negative | Negative | Negative |
| 12MAY2020 (29) | Negative | Negative | Indeterminate |
| 15SEP2020 (58) | Negative | Negative | Negative |
| 21JAN2021 (186) | Negative | Negative | Indeterminate |
| 09MAR2020 (29) | Negative | Negative | Indeterminate |
| 28APR2020 (79) | Negative | Negative | Negative |
| 21APR2020 (57) | Negative | Negative | Negative |
| 08DEC2020 (177) | Negative | Negative | Negative |
| 11AUG2020 (58) | Negative | Negative | Negative |
| 08SEP2020 (57) | Negative | Negative | Negative |
| 20JAN2021 (191) | Negative | Negative | Indeterminate |

TABLE 7

Listing of MRD Data; Cohort B All Treated Analysis Set

| | | | |
|---|---|---|---|
| Date of MRD Assessment (Study Day) | MRD Status at 10⁻⁴ Level | MRD Status at 10⁻⁵ Level | MRD Status at 10⁻⁶ Level |
| 03NOV2020 (58) | Negative | Negative | Negative |
| 16NOV2020 (35) | Negative | Negative | Indeterminate |
| 29DEC2020 (78) | Negative | Negative | Indeterminate |
| 27JAN2021 (57) | Negative | Negative | Negative |
| 09NOV2020 (56) | Negative | Negative | Negative |
| 15MAR2021 (182) | Negative | Negative | Negative |
| 01FEB2021 (56) | Negative | Negative | Indeterminate |
| 29MAR2021 (56) | Negative | Negative | Negative |
| 16NOV2020 (56) | Negative | Negative | Negative |
| 16FEB2021 (359) | Negative | Negative | Positive |
| 21APR2020 (58) | Negative | Negative | Negative |
| 12JAN2021 (86) | Negative | Negative | Negative |
| 15DEC2020 (58) | Negative | Negative | Indeterminate |

TABLE 8

Listing of MRD Data; Cohort C All Treated Analysis Set

| | | | |
|---|---|---|---|
| Date of MRD Assessment (Study Day) | MRD Status at 10⁻⁴ Level | MRD Status at 10⁻⁵ Level | MRD Status at 10⁻⁶ Level |
| 08MAR2021 (55) | Positive | Positive | Positive |
| 22DEC2020 (58) | Negative | Negative | Indeterminate |
| 03NOV2020 (56) | Negative | Positive | Positive |
| 11MAR2021 (184) | Positive | Positive | Positive |
| 18DEC2020 (101) | Positive | Positive | Positive |
| 05MAR2021 (186) | Negative | Negative | Indeterminate |
| 26OCT2020 (56) | Negative | Indeterminate | Indeterminate |
| 04NOV2020 (58) | Negative | Negative | Negative |
| 17NOV2020 (57) | Negative | Negative | Negative |
| 23MAR2021 (183) | Negative | Positive | Positive |

TABLE 9

Summary of Overall MRD Negativity Rate in Bone Marrow; Cohort A All Treated Analysis Set

| | Total |
|---|---|
| Analysis set: all treated | 29 |
| MRD negativity rate (10⁻⁴) | 12 (41.4%) |
| Exact 95% CI of MRD negative rate | (23.5%, 61.1%) |
| MRD negativity rate (10⁻⁵) | 12 (41.4%) |
| Exact 95% CI of MRD negative rate | (23.5%, 61.1%) |
| MRD negativity rate (10⁻⁶) | 12 (41.4%) |
| Exact 95% CI of MRD negative rate | (23.5%, 61.1%) |

Key: CI = confidence interval, MRD = minimal residual disease.

TABLE 10

Summary of Overall MRD Negativity Rate in Bone Marrow; Cohort B All Treated Analysis Set

| | Total |
|---|---|
| Analysis set: all treated | 18 |
| MRD negativity rate (10⁻⁴) | 9 (50.0%) |
| Exact 95% CI of MRD negative rate | (26.0%, 74.0%) |
| MRD negativity rate (10⁻⁵) | 9 (50.0%) |
| Exact 95% CI of MRD negative rate | (26.0%, 74.0%) |
| MRD negativity rate (10⁻⁶) | 7 (38.9%) |
| Exact 95% CI of MRD negative rate | (17.3%, 64.3%) |

Key: CI = confidence interval, MRD = minimal residual disease.

TABLE 11

Summary of Overall MRD Negativity Rate in Bone Marrow; Cohort C All Treated Analysis Set

| | Total |
|---|---|
| Analysis set: all treated | 20 |
| MRD negativity rate (10⁻⁴) | 5 (25.0%) |
| Exact 95% CI of MRD negative rate | (8.7%, 49.1%) |
| MRD negativity rate (10⁻⁵) | 4 (20.0%) |
| Exact 95% CI of MRD negative rate | (5.7%, 43.7%) |
| MRD negativity rate (10⁻⁶) | 2 (10.0%) |
| Exact 95% CI of MRD negative rate | (1.2%, 31.7%) |

Key: CI = confidence interval, MRD = minimal residual disease.

TABLE 12

Summary of Overall MRD Negativity Rate at 10⁻⁵ in Bone Marrow; Cohort A Subjects with Evaluable Sample at 10⁻⁵ in All Treated Analysis Set

| | Total |
|---|---|
| Analysis set: subjects with evaluable sample at 10⁻⁵ in all treated | 13 |
| MRD negativity rate (10⁻⁵) | 12 (92.3%) |
| Exact 95% CI of MRD negative rate | (64.0%, 99.8%) |

Key: CI = confidence interval, MRD = minimal residual disease.

TABLE 13

Summary of Overall MRD Negativity Rate at 10⁻⁵ in Bone Marrow; Cohort B Subjects with Evaluable Sample at 10⁻⁵ in All Treated Analysis Set

| | Total |
|---|---|
| Analysis set: subjects with evaluable sample at 10⁻⁵ in all treated | 9 |
| MRD negativity rate (10⁻⁵) | 9 (100.0%) |
| Exact 95% CI of MRD negative rate | (66.4%, 100.0%) |

Key: CI = confidence interval, MRD = minimal residual disease.

TABLE 14

Summary of Overall MRD Negativity Rate at 10⁻⁵ in Bone Marrow; Cohort C Subjects with Evaluable Sample at 10⁻⁵ in All Treated Analysis Set

| | Total |
|---|---|
| Analysis set: subjects with evaluable sample at 10⁻⁵ in all treated | 6 |
| MRD negativity rate (10⁻⁵) | 4 (66.7%) |
| Exact 95% CI of MRD negative rate | (22.3%, 95.7%) |

Key: CI = confidence interval, MRD = minimal residual disease.

TABLE 15

Listing of Disease Set Response Assessment; Cohort A All Treated Analysis

| | | |
|---|---|---|
| First Response and Date of First Response (Study Day) | Best Confirmed Response and Date of Best Confirmed Response (Study Day) | Date of PD (Study Day) and Reason |
| CR/2020-05-26(30) | sCR/2020-06-23(58) | 2021-02- 22(302)/NEW BONE LES |
| CR/2020-07-23(32) | sCR/2020-12-22(184) | |
| VGPR/2020-09-01 (30) | sCR/2021-02-04(186) | |
| PR/2020-07-22(30) | CR/2020-11-24(155) | |
| sCR/2020-11-04(78) | sCR/2020-11-04(78) | |
| VGPR/2020-09-06(21) | sCR/2020-09-13(28) | |
| VGPR/2020-10-07(29) | sCR/2021-03-08(181) | |
| CR/2020-11-03(30) | sCR/2020-12-02(59) | |
| VGPR/2020-06-29(29) | sCR/2020-08-17(78) | |
| | NE | |
| | NE | |
| CR/2020-09-01 (30) | sCR/2020-09-29(58) | |
| | SD | |
| | MR/2020-05-27(30) | |
| PR/2020-05-12(29) | sCR/2020-12-09(240) | |
| VGPR/2020-08-17(29) | sCR/2020-11-19(123) | |
| | NE | |
| | SD | |
| VGPR/2020-03-09(29) | sCR/2020-05-18(99) | |
| VGPR/2020-03-24(29) | sCR/2020-05-12(78) | |
| VGPR/2020-09-22(100) | VGPR/2020-09-22(100) | |
| CR/2020-07-06(29) | sCR/2020-08-03(57) | |
| VGPR/2020-12-03(57) | VGPR/2020-12-03(57) NE | 2020-12-22(76)/PLASMACY INCR SIZE |
| | SD | 2021-04-13(56)/PLASMACY INCR SIZE |
| PR/2020-07-08(30) | sCR/2021-01-05(211) | |
| PR/2020-08-11 (29) | CR/2020-12-16(156) | |
| VGPR/2021-03-17(30) | VGPR/2021-03-17(30) | |
| | NE | |

Keys: CR = complete response; MR = minimal response; NE = not evaluable; PD = progressive disease; PR = partial response; SD = stable disease; sCR = stringent response;
VGPR = very good partial response.
* Subject received Cilta-cel infusion as an outpatient

TABLE 16

Listing of Disease Response Assessment; Cohort B All Treated Analysis Set

| First Response and Date of First Response (Study Day) | Best Confirmed Response and Date of Best Confirmed Response (Study Day) | Date of PD (Study Day) and Reason |
|---|---|---|
| PR/2020-10-05(29) | VGPR/2020-11-03(58) | 2021-03-10(185)/SPEP |
| VGPR/2020-12-21(78) | sCR/2021-03-08(155) | |
| PR/2021-02-10(28) | VGPR/2021-03-10(56) | |
| PR/2021-03-15(29) | PR/2021-03-15(29) SD | |
| PR/2020-12-29(28) | VGPR/2021-01-26(56) NE | |
| PR/2021-03-16(28) | PR/2021-03-16(28) | |
| PR/2020-10-12(28) | VGPR/2021-01-18(126) | |
| CR/2021-01-04(28) | CR/2021-01-04(28) | |
| VGPR/2021-03-01(28) | VGPR/2021-03-01(28) | |
| PR/2020-11-16(56) | PR/2020-11-16(56) | |
| PR/2020-12-08(30) | PR/2020-12-08(30) | |
| PR/2020-03-23(29) | sCR/2021-02-16(359) | |
| VGPR/2021-02-16(29) | VGPR/2021-02-16(29) | |
| VGPR/2020-11-17(30) | VGPR/2020-11-17(30) | |
| VGPR/2021-01-11(28) | sCR/2021-02-08(56) | |
| sCR/2020-12-01 (28) | sCR/2020-12-01 (28) | |

Keys: CR = complete response; MR = minimal response; NE = not evaluable; PD = progressive disease; PR = partial response; SD = stable disease; sCR = stringent response;
VGPR = very good partial response.
* Subject received Cilta-cel infusion as an outpatient

TABLE 17

Listing of Disease Response Assessment; Cohort C All Treated Analysis Set

| | | |
|---|---|---|
| First Response and Date of First Response (Study Day) | Best Confirmed Response and Date of Best Confirmed Response (Study Day) | Date of PD (Study Day) and Reason |
| | NE | 2020-12-15(30)/PLASMACY INCR SIZE |
| PR/2020-07-08(31) | SD sCR/2020-11-29(175) | |
| PR/2020-11-22(28) | VGPR/2020-12-22(58) | |
| | PD/2021-02-10(15) | 2021-02-10(15)/FLC |
| | MR/2020-11-16(28) SD | |
| | PD/2021-01-11(28) NE | 2021-01-11(28)/UPEP |
| PR/2020-10-06(28) | NE CR/2020-11-24(77) | |
| PR/2020-09-30(30) | SD VGPR/2020-10-26(56) | |
| PR/2020-12-09(29) | VGPR/2021-01-04(55) SD | 2020-06-29(78)/FLC |
| sCR/2021-02-09(155) | sCR/2021-02-09(155) SD | 2020-11-10(44)/FLC |
| VGPR/2020-10-26(27) | VGPR/2020-10-26(27) SD | |
| PR/2020-12-03(28) | PR/2020-12-03(28) | |
| First Response and Date of First Response (Study Day) | Best Confirmed Response and Date of Best Confirmed Response (Study Day) | Date of PD (Study Day) and Reason |

Keys: CR = complete response; MR = minimal response; NE = not evaluable; PD = progressive disease; PR = partial response; SD = stable disease; sCR = stringent response;
VGPR = very good partial response.
* Subject received Cilta-cel infusion as an outpatient

TABLE 18

Overall Best Response Assessment; Cohort A All Treated Analysis Set
Total

| | n (%) | Exact 95% CI for % |
|---|---|---|
| Analysis set: all treated | 29 | |

| | | |
|---|---|---|
| Best response | | |
| Stringent complete response (sCR) | 15 (51.7%) | (32.5%, 70.6%) |
| MRD-negative CR/sCR | 10 (34.5%) | (17.9%, 54.3%) |
| Complete response (CR) | 2 (6.9%) | (0.8%, 22.8%) |
| Very good partial response (VGPR) | 3 (10.3%) | (2.2%, 27.4%) |
| Partial response (PR) | 0 | (NE, NE) |
| Minimal response (MR) | 1 (3.4%) | (0.1%, 17.8%) |
| Stable disease (SD) | 3 (10.3%) | (2.2%, 27.4%) |
| Progressive disease (PD) | 0 | (NE, NE) |
| Not evaluable (NE) | 5 (17.2%) | (5.8%, 35.8%) |
| Overall response (sCR + CR + VGPR + PR) | 20 (69.0%) | (49.2%, 84.7%) |
| Clinical benefit (Overall response + MR) | 21 (72.4%) | (52.8%, 87.3%) |
| VGPR or better (sCR + CR + VGPR) | 20 (69.0%) | (49.2%, 84.7%) |
| CR or better (sCR + CR) | 17 (58.6%) | (38.9%, 76.5%) |

TABLE 19

Overall Best Response Assessment; Cohort B All Treated Analysis Set
Total

| | n (%) | Exact 95% CI for % |
|---|---|---|
| Analysis set: all treated | 18 | |
| Best response | | |
| Stringent complete response (sCR) | 4 (22.2%) | (6.4%, 47.6%) |
| MRD-negative CR/sCR | 2 (11.1%) | (1.4%, 34.7%) |
| Complete response (CR) | 1 (5.6%) | (0.1%, 27.3%) |
| Very good partial response (VGPR) | 7 (38.9%) | (17.3%, 64.3%) |
| Partial response (PR) | 4 (22.2%) | (6.4%, 47.6%) |
| Minimal response (MR) | 0 | (NE, NE) |
| Stable disease (SD) | 1 (5.6%) | (0.1%, 27.3%) |
| Progressive disease (PD) | 0 | (NE, NE) |
| Not evaluable (NE) | 1 (5.6%) | (0.1%, 27.3%) |
| Overall response (sCR + CR + VGPR + PR) | 16 (88.9%) | (65.3%, 98.6%) |
| Clinical benefit (Overall response + MR) | 16 (88.9%) | (65.3%, 98.6%) |
| VGPR or better (sCR + CR + VGPR) | 12 (66.7%) | (41.0%, 86.7%) |
| CR or better (sCR + CR) | 5 (27.8%) | (9.7%, 53.5%) |

TABLE 20

Overall Best Response Assessment; Cohort C All Treated Analysis Set
Total

| | n (%) | Exact 95% CI for % |
|---|---|---|
| Analysis set: all treated | 20 | |
| Best response | | |
| Stringent complete response (sCR) | 2 (10.0%) | (1.2%, 31.7%) |
| MRD-negative CR/sCR | 0 | (NE, NE) |
| Complete response (CR) | 1 (5.0%) | 0.1% 24.9%) |
| Very good partial response (VGPR) | 4 (20.0%) | (5.7%, 43.7%) |
| Partial response (PR) | 1 (5.0%) | (0.1%, 24.9%) |
| Minimal response (MR) | 1 (5.0%) | (0.1%, 24.9%) |
| Stable disease (SD) | 6 (30.0%) | (11.9%, 54.3%) |
| Progressive disease (PD) | 2 (10.0%) | (1.2%, 31.7%) |
| Not evaluable (NE) | 3 (15.0%) | (3.2%, 37.9%) |
| Overall response (sCR + CR + VGPR + PR) | 8 (40.0%) | (19.1%, 63.9%) |
| Clinical benefit (Overall response + MR) | 9 (45.0%) | (23.1%, 68.5%) |
| VGPR or better (sCR + CR + VGPR) | 7 (35.0%) | (15.4%, 59.2%) |
| CR or better (sCR + CR) | 3 (15.0%) | (3.2%, 37.9%) |

TABLE 21

Descriptive Summaries for Time to Response Assessment; Cohort A Responders in All Treated Analysis Set

| | Total |
|---|---|
| Analysis set: responders in all treated | 20 |
| Time to first response^{a} (months) | |
| N | 20 |
| Mean (SD) | 1.20 (0.639) |
| Median | 0.99 |
| Range | (0.7; 3.3) |
| Time to best response (months) | |
| N | 20 |
| Mean (SD) | 3.64 (2.101) |
| Median | 2.91 |
| Range | (0.9; 7.9) |
| Time to CR or better (months) | |
| N | 17 |
| Mean (SD) | 2.70 (1.938) |
| Median | 2.56 |
| Range | (0.9; 7.9) |

Key: CR = complete response
^{a}Response = PR or better

TABLE 22

Descriptive Summaries for Time to Response Assessment; Cohort B Responders in All Treated Analysis Set

| | Total |
|---|---|
| Analysis set: responders in all treated | 16 |
| Time to first response^{a} (months) | |
| N | 16 |
| Mean (SD) | 1.10 (0.452) |
| Median | 0.94 |
| Range | (0.9; 2.6) |
| Time to best response (months) | |
| N | 16 |
| Mean (SD) | 2.37 (2.792) |
| Median | 1.41 |
| Range | (0.9; 11.8) |
| Time to CR or better (months) | |
| N | 5 |
| Mean (SD) | 4.07 (4.542) |
| Median | 1.84 |
| Range | (0.9; 11.6) |

Key: CR = complete response
^{a}Response PR or better

TABLE 23

Descriptive Summaries for Time to Response Assessment; Cohort C Responders in All Treated Analysis Set

| | Total |
|---|---|
| Analysis set: responders in all treated | 8 |
| Time to first response^{a} (months) | |
| N | 8 |
| Mean (SD) | 1.46 (1.467) |
| Median | 0.94 |
| Range | (0.9; 5.1) |
| Time to best response (months) | |
| N | 8 |
| Mean (SD) | 2.59 (1.835) |
| Median | 1.87 |
| Range | (0.9; 5.7) |
| Time to CR or better (months) | |
| N | 3 |
| Mean (SD) | 4.46 (1.701) |
| Median | 5.09 |
| Range | (2.5; 5.7) |

Key: CR = complete response
^{a}Response PR or better

TABLE 24

Duration of Response Assessment; Cohort A Responders in All Treated Analysis Set

| | Total |
|---|---|
| Analysis set: responders in all treated | 20 |
| Duration of response | |
| Number of events (%) | 2 (10.0%) |
| Number of censored (%) | 18 (90.0%) |
| Kaplan-Meier estimate (months) | |
| 25% quantile (95% CI) | 9.0 (0.7, NE) |
| Median (95% CI) | NE (9.0, NE) |
| 75% quantile (95% CI) | NE (9.0, NE) |
| 6-month event-free rate % (95% CI) | 95.0 (69.5, 99.3) |
| 9-month event-free rate % (95% CI) | 63.3 (6.9, 92.5) |
| 12-month event-free rate % (95% CI) | NE (NE, NE) |

Keys: CI = confidence interval, NE = Not estimable.

TABLE 25

Duration of Response Assessment; Cohort B Responders in All Treated Analysis Set

| | Total |
|---|---|
| Analysis set: responders in all treated | 16 |
| Duration of response | |
| Number of events (%) | 1 (6.3%) |
| Number of censored (%) | 15 (93.8%) |
| Kaplan-Meier estimate (months) | |
| 25% quantile (95% CI) | 5.2 (5.2, NE) |
| Median (95% CI) | NE (5.2, NE) |
| 75% quantile (95% CI) | NE (5.2, NE) |
| 6-month event-free rate % (95% CI) | 66.7 (5.4, 94.5) |
| 9-month event-free rate % (95% CI) | 66.7 (5.4, 94.5) |
| 12-month event-free rate % (95% CI) | 66.7 (5.4, 94.5) |

Keys: CI = confidence interval, NE = Not estimable.

TABLE 26

Duration of Response Assessment; Cohort C Responders in All Treated Analysis Set

| | Total |
|---|---|
| Analysis set: responders in all treated | 8 |
| Duration of response | |
| Number of events (%) | 1 (12.5%) |
| Number of censored (%) | 7 (87.5%) |
| Kaplan-Meier estimate (months) | |
| 25% quantile (95% CI) | NE (4.4, NE) |
| Median (95% CI) | NE (4.4, NE) |
| 75% quantile (95% CI) | NE (4.4, NE) |
| 6-month event-free rate % (95% CI) | 80.0 (20.4, 96.9) |
| 9-month event-free rate % (95% CI) | NE (NE, NE) |
| 12-month event-free rate % (95% CI) | NE (NE, NE) |

Keys: CI = confidence interval, NE = Not estimable.

TABLE 27

Progression-Free Survival Assessment; Cohort A All Treated Analysis Set

| | Total |
|---|---|
| Analysis set: all treated | 29 |
| Progression-free survival | |
| Number of events (%) | 4 (13.8%) |
| Number of censored (%) | 25 (86.2%) |
| Kaplan-Meier estimate (months) | |
| 25% quantile (95% CI) | 9.92 (1.84, NE) |
| Median (95% CI) | NE (9.92, NE) |
| 75% quantile (95% CI) | NE (9.92, NE) |
| 6-month progression-free survival rate % (95% CI) | 85.9 (62.4, 95.2) |
| 9-month progression-free survival rate % (95% CI) | 85.9 (62.4, 95.2) |
| 12-month progression-free survival rate % (95% CI) | NE (NE, NE) |
| 18-month progression-free survival rate % (95% CI) | NE (NE, NE) |

Key: CI = confidence interval

TABLE 28

Progression-Free Survival Assessment; Cohort B All Treated Analysis Set

| | Total |
|---|---|
| Analysis set: all treated | 18 |
| Progression-free survival | |
| Number of events (%) | 1 (5.6%) |
| Number of censored (%) | 17 (94.4%) |
| Kaplan-Meier estimate (months) | |
| 25% quantile (95% CI) | 6.08 (6.08, NE) |
| Median (95% CI) | NE (6.08, NE) |
| 75% quantile (95% CI) | NE (6.08, NE) |
| 6-month progression-free survival rate % (95% CI) | 100.0 (100.0, 100.0) |
| 9-month progression-free survival rate % (95% CI) | 66.7 (5.4, 94.5) |
| 12-month progression-free survival rate % (95% CI) | 66.7 (5.4, 94.5) |
| 18-month progression-free survival rate % (95% CI) | NE (NE, NE) |

Key: CI = confidence interval

TABLE 29

Progression-Free Survival Assessment; Cohort C All Treated Analysis Set

| | Total |
|---|---|
| Analysis set: all treated | 20 |
| Progression-free survival | |
| Number of events (%) | 8 (40.0%) |
| Number of censored (%) | 12 (60.0%) |
| Kaplan-Meier estimate (months) | |
| 25% quantile (95% CI) | 1.45 (0.49, 5.29) |
| Median (95% CI) | NE (1.45, NE) |
| 75% quantile (95% CI) | NE (NE, NE) |
| 6-month progression-free survival rate % (95% CI) | 54.9 (28.8, 74.9) |
| 9-month progression-free survival rate % (95% CI) | 54.9 (28.8, 74.9) |
| 12-month progression-free survival rate % (95% CI) | NE (NE, NE) |
| 18-month progression-free survival rate % (95% CI) | NE (NE, NE) |
| Key: CI = confidence interval | |

TABLE 30

Cytokine Release Syndrome ASTCT Consensus Grading System

| | |
|---|---|
| Grade | Toxicity |
| Grade 1 | Fever^{a} (Temperature ≥38°) |
| Grade 2 | Fever^{a} (Temperature ≥38°) with either: • Hypotension not requiring vasopressors • And/or^{c} hypoxia requiring low-flow nasal cannula^{b} or blow-by. |
| Grade 3 | Fever^{a} (Temperature ≥38°) with either: • Hypotension requiring a vasopressor with or without vasopressin, • And/or^{c} hypoxia requiring high-flow nasal cannula^{b}, facemask, nonrebreather mask, or Venturi mask. |
| Grade 4 | Fever^{a} (Temperature ≥38°) with either: • hypotension requiring multiple vasopressors (excluding vasopressin), • And/or^{c} hypoxia requiring positive pressure (e.g., CPAP, BiPAP, intubation and mechanical ventilation). |
| Grade 5 | Death |

^{a} Fever not attributable to any other cause. In patients who have CRS then receive antipyretics or anticytokine therapy such as tocilizumab or steroids, fever is no longer required to grade subsequent CRS severity. In this case, CRS grading is driven by hypotension and/or hypoxia.
^{b} Low-flow nasal cannula is defined as oxygen delivered at ≤6 L/minute or blow-by oxygen delivery. High-flow nasal cannula is defined as oxygen delivered at >6 L/minute.
^{c} CRS grade is determined by the more severe event: hypotension or hypoxia not attributable to any other cause.
Note: Organ toxicities associated with CRS may be graded according to CTCAE v5.0 but they do not influence CRS grading.
Source: Lee (2019)

TABLE 31

Immune Effector Cell-associated Neurotoxicity Syndrome (ICANS) ASTCT Consensus Grading System^{a,b}

| | | | | |
|---|---|---|---|---|
| Neurotoxicity Domain | Grade 1 | Grade 2 | Grade 3 | Grade 4 |
| ICE Score | 7-9 | 3-6 | 0-2 | 0 (patient is unarousable and unable to perform ICE). |
| Depressed Level of Consciousness | Awakens spontaneously. | Awakens to voice. | Awakens only to tactile stimulus. | Patient is unarousable or requires vigorous or repetitive tactile stimuli to arouse. Stupor or coma. |
| Seizure | N/A | N/A | Any clinical seizure, focal or generalized, that resolves rapidly; or Non-convulsive seizures on EEG that resolve with intervention. | Life-threatening prolonged seizure (>5 min); or Repetitive clinical or electrical seizures without return to baseline in between. |
| Motor Findings | N/A | N/A | N/A | Deep focal motor weakness such as hemiparesis or paraparesis. |
| Raised Intracranial Pressure / Cerebral Edema | N/A | N/A | Focal/local edema on neuroimaging. | Diffuse cerebral edema on neuroimaging; or Decerebrate or decorticate posturing; or Cranial nerve VI palsy; or Papilledema; or Cushing’s triad. |

a: Toxicity grading according to Lee et al 2019
b: ICANS grade is determined by the most severe event (ICE score, level of consciousness, seizure, motor findings, raised ICP/cerebral edema) not attributable to any other cause.
Note: all other neurological adverse events (not associated with ICANS) should continue to be graded with CTCAE Version 5.0 during both phases of the study

TABLE 32

Prior anti-BCMA exposure and response to cilta-cel; Cohort C All Treated Analysis Set

| | Prior Bispecific Antibody | | Prior ADC | |
|---|---|---|---|---|
| | Cilta-cel Responders | Cilta-cel Non-responders | Cilta-cel Responders | Cilta-cel Non-responders |
| Duration of anti-BCMA tx, days | | | | |
| Median | 53.5 | 130.0 | 22.0 | 63.0 |
| Mean | 64.3 | 139.7 | 53.9 | 150.4 |
| Min, Max | 23, 127 | 29, 260 | 1, 277 | 44, 527 |
| Time from anti-BCMA tx to apheresis, days | | | | |
| Median | 219.5 | 83.0 | 149.0 | 56.0 |
| Mean | 186.5 | 136.3 | 263.6 | 234.2 |
| Min, Max | 27, 280 | 76, 250 | 25, 694 | 39, 894 |
| Time from anti-BCMA tx and cilta-cel infusion, days | | | | |
| Median | 275.0 | 123.0 | 210.5 | 115.0 |
| Mean | 240.3 | 182.3 | 323.1 | 291.8 |
| Min, Max | 83, 328 | 118, 306 | 61, 748 | 94, 943 |

Key: ADC, antibody drug-conjugate; BCMA, B-cell maturation antigen; tx, treatment

### SEQUENCES

### Seq Id No: 1 - Ciltacabtagene Autoleucel CAR CD8a Signal Peptide, CD8a SP Amino Acid Sequence

MALPVTALLLPLALLLHAARP

### Seq Id No: 2 - Ciltacabtagene Autoleucel CAR BCMA Binding Domain, VHH1 Amino Acid Sequence

QVKLEESGGGLVQAGRSLRLSCAASEHTFSSHVMGWFRQAPGKERESVAV
IGWRDISTSYADSVKGRFTISRDNAKKTLYLQMNSLKPEDTAVYYCAARR
IDAADFDSWGQGTQVTVSS

### Seq Id No: 3 - Ciltacabtagene Autoleucel CAR BCMA Binding Domain, G4S Linker Amino Acid Sequence

GGGGS

### Seq Id No: 4 - Ciltacabtagene Autoleucel CAR BCMA Binding Domain, VHH2 Amino Acid Sequence

EVQLVESGGGLVQAGGSLRLSCAASGRTFTMGWFRQAPGKEREFVAAISL
SPTLAYYAESVKGRFTISRDNAKNTVVLQMNSLKPEDTALYYCAADRKSV
MSIRPDYWGQGTQVTVSS

### Seq Id No: 5 - Ciltacabtagene Autoleucel CAR CD8a Hinge Amino Acid Sequence

TTTPAPRPPTPAPTIASQPLSLRPEACRPAAGGAVHTRGLDFACD

### Seq Id No: 6 - Ciltacabtagene Autoleucel CAR CD8a Transmembrane Amino Acid Sequence

IYIWAPLAGTCGVLLLSLVITLYC

### Seq Id No: 7 - Ciltacabtagene Autoleucel CAR CD137 Cytoplasmic Amino Acid Sequence

KRGRKKLLYIFKQPFMRPVQTTQEEDGCSCRFPEEEEGGCEL

### Seq Id No: 8 - Ciltacabtagene Autoleucel CAR CD3z Cytoplasmic Amino Acid Sequence

RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

### Seq Id No: 9 - Ciltacabtagene Autoleucel CAR CD8a Signal Peptide CD8a SP Nucleic Acid Sequence

ATGGCTCTGCCCGTCACCGCTCTGCTGCTGCCTCTGGCTCTGCTGCTGCA
CGCTGCTCGCCCT

### Seq Id No: 10 - Ciltacabtagene Autoleucel CAR BCMA Binding Domain, VHH1 Nucleic Acid Sequence

CAGGTCAAACTGGAAGAATCTGGCGGAGGCCTGGTGCAGGCAGGACGGAG
CCTGCGCCTGAGCTGCGCAGCATCCGAGCACACCTTCAGCTCCCACGTGA
TGGGCTGGTTTCGGCAGGCCCCAGGCAAGGAGAGAGAGAGCGTGGCCGTG
ATCGGCTGGAGGGACATCTCCACATCTTACGCCGATTCCGTGAAGGGCCG
GTTCACCATCAGCCGGGACAACGCCAAGAAGACACTGTATCTGCAGATGA
ACAGCCTGAAGCCCGAGGACACCGCCGTGTACTATTGCGCAGCAAGGAGA
ATCGACGCAGCAGACTTTGATTCCTGGGGCCAGGGCACCCAGGTGACAGT
GTCTAGC

### Seq Id No: 11 - Ciltacabtagene Autoleucel CAR BCMA Binding Domain, G4S Linker Nucleic acid Sequence

GGAGGAGGAGGATCT

### Seq Id No: 12 - Ciltacabtagene Autoleucel CAR BCMA Binding Domain, VHH2 Nucleic Acid Sequence

GAGGTGCAGCTGGTGGAGAGCGGAGGCGGCCTGGTGCAGGCCGGAGGCTC
TCTGAGGCTGAGCTGTGCAGCATCCGGAAGAACCTTCACAATGGGCTGGT
TTAGGCAGGCACCAGGAAAGGAGAGGGAGTTCGTGGCAGCAATCAGCCTG
TCCCCTACCCTGGCCTACTATGCCGAGAGCGTGAAGGGCAGGTTTACCAT
CTCCCGCGATAACGCCAAGAATACAGTGGTGCTGCAGATGAACTCCCTGA
AACCTGAGGACACAGCCCTGTACTATTGTGCCGCCGATCGGAAGAGCGTG
ATGAGCATTAGACCAGACTATTGGGGGCAGGGAACACAGGTGACCGTGAG
CAGC

### Seq Id No: 13 - Ciltacabtagene Autoleucel CAR CD8a Hinge Nucleic Acid Sequence

ACCACGACGCCAGCGCCGCGACCACCAACACCGGCGCCCACCATCGCGTC
GCAGCCCCTGTCCCTGCGCCCAGAGGCGTGCCGGCCAGCGGCGGGGGGCG
CAGTGCACACGAGGGGGCTGGACTTCGCCTGTGAT

### Seq Id No: 14 - Ciltacabtagene Autoleucel CAR CD8a Transmembrane Nucleic Acid Sequence

ATCTACATCTGGGCGCCCTTGGCCGGGACTTGTGGGGTCCTTCTCCTGTC
ACTGGTTATCACCCTTTACTGC

### Seq Id No: 15 - Ciltacabtagene Autoleucel CAR CD137 Cytoplasmic Nucleic Acid Sequence

AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG

### Seq Id No: 16 - Ciltacabtagene Autoleucel CAR CD3z Cytoplasmic Nucleic Acid Sequence

AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA

### Seq Id No: 17 - Ciltacabtagene Autoleucel CAR Amino Acid Sequence

MALPVTALLLPLALLLHAARPQVKLEESGGGLVQAGRSLRLSCAASEHTF
SSHVMGWFRQAPGKERESVAVIGWRDISTSYADSVKGRFTISRDNAKKTL
YLQMNSLKPEDTAVYYCAARRIDAADFDSWGQGTQVTVSSGGGGSEVQLV
ESGGGLVQAGGSLRLSCAASGRTFTMGWFRQAPGKEREFVAAISLSPTLA
YYAESVKGRFTISRDNAKNTVVLQMNSLKPEDTALYYCAADRKSVMSIRP
DYWGQGTQVTVSSTSTTTPAPRPPTPAPTIASQPLSLRPEACRPAAGGAV
HTRGLDFACDIYIWAPLAGTCGVLLLSLVITLYCKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR

### Seq Id No: 18 - Ciltacabtagene Autoleucel CAR BCMA Binding Domain, VHH1 CDR1 Amino acid Sequence

SHVMG

### Seq Id No: 19 - Ciltacabtagene Autoleucel CAR BCMA Binding Domain, VHH1 CDR2 Amino acid Sequence

VIGWRDISTSYADSVKG

### Seq Id No: 20 - Ciltacabtagene Autoleucel CAR BCMA Binding Domain, VHH1 CDR3 Amino acid Sequence

ARRIDAADFDS

### Seq Id No: 21 - Ciltacabtagene Autoleucel CAR BCMA Binding Domain, VHH2 CDR1 Amino acid Sequence

TFTMG

### Seq Id No: 22 - Ciltacabtagene Autoleucel CAR BCMA Binding Domain, VHH2 CDR2 Amino acid Sequence

AISLSPTLAYYAESVKG

### Seq Id No: 23 - Ciltacabtagene Autoleucel CAR BCMA Binding Domain, VHH2 CDR3 Amino acid Sequence

ADRKSVMSIRPDY

1. A method of treating a subject, said method comprising administering to the subject a composition comprising a therapeutically effective number of T cells comprising a chimeric antigen receptor (CAR) comprising:
(a) an extracellular antigen binding domain comprising:
(1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
(2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain, to deliver to the subject a dose of CAR expressing T cells (CAR-T cells), wherein said subject:
(i) has multiple myeloma;
(ii) has received prior treatment with one, two or three prior lines of therapy; and
(iii) is lenalidomide-refractory;
optionally wherein:
(1) the subject received prior treatment with dexamethasone, an alkylating agent or daratumumab;
(2) the multiple myeloma is refractory to the last line of therapy;
(3) the subject has relapsed after said one, two or three prior lines of therapy;
(4) the multiple myeloma is refractory to three classes of medicaments; or
(5) said method is effective in obtaining a rate of immune-effector cell associated neurotoxicity of between approximately 20% and approximately 99%.

2. A method of treating a subject, said method comprising administering to the subject a composition comprising a therapeutically effective number of T cells comprising a chimeric antigen receptor (CAR) comprising:
(a) an extracellular antigen binding domain comprising:
(1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
(2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain, to deliver to the subject a dose of CAR expressing T cells (CAR-T cells), wherein said subject:
(i) has multiple myeloma;
(ii) has received prior treatment with one prior line of therapy, said one prior line of therapy comprising treatment with at least two medicaments, said at least two medicaments comprising a proteasomal inhibitor and an immunomodulatory drug; and
(iii) has had a prior early relapse.

3. A method of treating a subject, said method comprising administering to the subject a composition comprising a therapeutically effective number of T cells comprising a chimeric antigen receptor (CAR) comprising:
(a) an extracellular antigen binding domain comprising:
(1) a first anti-BCMA binding moiety comprising a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; and
(2) a second BCMA binding moiety comprising a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23;
(b) a transmembrane domain; and
(c) an intracellular signaling domain, to deliver to the subject a dose of CAR expressing T cells (CAR-T cells), wherein said subject:
(i) has multiple myeloma; and
(ii) has received at least one prior line of therapy comprising treatment with at least four medicaments, said at least four medicaments comprising a non-cellular BCMA-targeting medicament.

4. The method of claim 1, wherein the subject received prior treatment with at least one prior line of therapy comprising treatment with lenalidomide and at least one non-lenalidomide medicament, said at least one non-lenalidomide medicament comprising at least one of:
(a) a proteasomal inhibitor;
(b) an immunomodulatory drug; or
(c) an anti-CD38 antibody;
optionally wherein the subject received prior treatment with at least two prior lines of therapy and optionally wherein the subject received prior treatment with three prior lines of therapy.

5-10. (canceled)

11. The method of claim 1, wherein:
(i) said method is effective in obtaining minimal residual disease (MRD) negative status in said subject assessed in the bone marrow after said administration of said CAR-T cells, optionally:
(1) wherein said minimal residual disease (MRD) negative status is obtained at a first follow-up time of between approximately 29 days and approximately 184 days after said administration of said CAR-T cells, further optionally wherein said method is effective in maintaining said minimal residual disease (MRD) negative status in said subject assessed in the bone marrow at a second follow-up time of between approximately 57 days and approximately 191 days after said administration of said CAR-T cells, further optionally wherein said first follow-up time is earlier than said second follow-up time;
(2) wherein said method is effective in obtaining said minimal residual disease (MRD) negative status at a rate of between approximately 24% and approximately 61% at a sensitivity threshold level of 10⁻⁴, 10⁻⁵ or 10⁻⁶;
(3) wherein said method is effective in obtaining said minimal residual disease (MRD) negative status at a rate of approximately 41% at a sensitivity threshold level of 10⁻⁴, 10⁻⁵ or 10⁻⁶;
(4) wherein said method is effective in obtaining said minimal residual disease (MRD) negative status at a rate of between approximately 64% and approximately 99% in subjects with evaluable samples at a sensitivity threshold level of 10⁻⁵; or
(5) wherein said method is effective in obtaining said minimal residual disease (MRD) negative status at a rate of approximately 92% in subjects with evaluable samples at a sensitivity threshold level of 10⁻⁵; or
(ii) said method further comprises treating said subject for cytokine release syndrome more than approximately 3 days after said administration of said CAR-T cells, optionally wherein said method is effective in obtaining a rate of recovery from said cytokine release syndrome of between approximately 1% and approximately 90% at a time of approximately 7 days after first observance of said cytokine release syndrome, further optionally:
(1) wherein said method further comprises treating said subject for cytokine release syndrome more than approximately 3 days following said administration of said CAR-T cells without significantly reducing expansion of said CAR-T cells in vivo; or
(2) wherein said treatment of cytokine release syndrome comprises administering an IL-6R inhibitor to the subject, further optionally wherein said IL-6R inhibitor is an antibody, further optionally wherein said antibody inhibits IL-6R by binding its extracellular domain, further optionally wherein said IL-6R inhibitor prevents the binding of IL-6 to IL-6R, and further optionally wherein the IL-6R inhibitor is tocilizumab.

12-17. (canceled)

18. The method of claim 1, wherein said method is effective in obtaining at least one response in the subject after said administration of said CAR-T cells, wherein said at least one response comprises, in order from better to worse:
(i) a stringent complete response;
(ii) a complete response;
(iii) a very good partial response;
(iv) a partial response; or
(v) a minimal response;
optionally:
(1) wherein said method is effective in maintaining a response at a rate of between approximately 70% and approximately 99% at a follow-up time of approximately 6 months after said administration of said CAR-T cells;
(2) wherein said method is effective in maintaining a response at a rate of approximately 95% at a follow-up time of approximately 6 months after said administration of said CAR-T cells;
(3) wherein said method is effective in maintaining a response at a rate of between approximately 7% and approximately 92% at a follow-up time of approximately 9 months after said administration of said CAR-T cells; or
(4) wherein said method is effective in maintaining a response at a rate of approximately 63% at a follow-up time of approximately 9 months after said administration of said CAR-T cells.

19. The method of claim 18, wherein:
(i) said method is effective in obtaining a first response before a time of between approximately 21 days and approximately 99 days after said administration of said CAR-T cells;
(ii) said method is effective in obtaining a first response before a time of between approximately 21 days and approximately 55 days after said administration of said CAR-T cells;
(iii) said method is effective in obtaining a first response before approximately 36 days after said administration of said CAR-T cells;
(iv) said method is effective in obtaining a first response before approximately 30 days after said administration of said CAR-T cells;
(v) said method is effective in obtaining a best response of any one of minimal response, partial response, very good partial response, complete response or stringent complete response, optionally wherein said method is effective in obtaining said best response of any one of minimal response, partial response, very good partial response, complete response or stringent complete response at a rate of between approximately 52% and approximately 87%, and optionally wherein said method is effective in obtaining said best response of any one of minimal response, partial response, very good partial response, complete response or stringent complete response at a rate of approximately 72%;
(vi) said method is effective in obtaining a best response of any one of partial response, very good partial response, complete response or stringent complete response, optionally wherein said method is effective in obtaining said best response of any one of partial response, very good partial response, complete response or stringent complete response at a rate of between approximately 49% and approximately 84%, and optionally said method is effective in obtaining said best response of any one of partial response, very good partial response, complete response or stringent complete response at a rate of approximately 69%;
(vii) said method is effective in obtaining a best response of any one of very good partial response, complete response or stringent complete response, optionally wherein said method is effective in obtaining said best response of any one of very good partial response, complete response or stringent complete response at a rate of between approximately 49% and approximately 84%, and optionally wherein said method is effective in obtaining said best response of any one of partial response, very good partial response, complete response or stringent complete response at a rate of approximately 69%;
(viii) said method is effective in obtaining a best response of complete response or stringent complete response, optionally wherein said method is effective in obtaining said best response of complete response or stringent complete response at a rate of between approximately 39% and approximately 76%, and optionally wherein said method is effective in obtaining said best response of complete response or stringent complete response at a rate of approximately 58%; or
(ix) said method is effective in obtaining a best response of stringent complete response, optionally wherein said method is effective in obtaining said best response of stringent complete response at a rate of between approximately 33% and approximately 70%, and optionally wherein said method is effective in obtaining said best response of stringent complete response at a rate of approximately 52%;
optinally:
(1) wherein said method is effective in obtaining said best response before a time of between approximately 27 days and approximately 237 days after said administration of said CAR-T cells;
(2) wherein said method is effective in obtaining said best response before a time of between approximately 46 days and approximately 172 days after said administration of said CAR-T cells;
(3) wherein said method is effective in obtaining said best response before approximately 109 days after said administration of said CAR-T cells;
(4) wherein said method is effective in obtaining said best response before approximately 87 days after said administration of said CAR-T cells; or
(5) wherein said method is effective in maintaining a response in the subject at a follow-up time between the time of said first response and approximately 270 days after said administration of said CAR-T cells;
further optionally wherein said method is effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells, further optionally wherein said method is effective in obtaining either minimal residual disease (MRD) negative complete response or minimal residual disease (MRD) negative stringent complete response at a rate of between approximately 18% and approximately 54% at a follow-up time of approximately 291 days after said administration of said CAR-T cells, and further optionally wherein said method is effective in obtaining either minimal residual disease (MRD) negative complete response or minimal residual disease (MRD) negative stringent complete response at a rate of approximately 35% at a follow-up time of approximately 291 days after said administration of said CAR-T cells.

20-37. (canceled)

38. The method of claim 1, wherein said method is effective in obtaining progression-free survival of the subject, optionally:
(i) wherein said method is effective in obtaining said progression-free survival of the subject at a time between said administration of said CAR-T cells and approximately 55 days after said administration of said CAR-T cells;
(ii) wherein said method is effective in obtaining said progression-free survival of the subject at a time between said administration of said CAR-T cells and approximately 297 days after said administration of said CAR-T cells;
(iii) wherein said method is effective in obtaining said progression-free survival at a rate of between approximately 62% and approximately 95% at a follow-up time of approximately 6 months or approximately 9 months after said administration of said CAR-T cells; or
(iv) wherein said method is effective in obtaining said progression-free survival at a rate of approximately 86% at a follow-up time of approximately 6 months or approximately 9 months after said administration of said CAR-T cells.

39-57. (canceled)

58. The method of claim 2, wherein:
(i) the subject was additionally treated with an anti-CD38 antibodys;
(ii) the multiple myeloma is refractory to at least one medicament; or
(iii) said method is effective in obtaining minimal residual disease (MRD) negative status in said subject assessed in the bone marrow after said administration of said CAR-T cells, optionally:
(1) wherein said minimal residual disease (MRD) negative status is assessed in the bone marrow at a first follow-up time at a first follow-up time of between approximately 35 days and approximately 58 days after said administration of said CAR-T cells, further optionally wherein said method is effective in maintaining said minimal residual disease (MRD) negative status in said subject assessed in the bone marrow at a second follow-up time of between approximately 78 days and approximately 359 days after said administration of said CAR-T cells, further wherein said first follow-up time is earlier than said second follow-up time;
(2) wherein said method is effective in obtaining said minimal residual disease (MRD) negative status at a rate of between approximately 26% and approximately 74% at a sensitivity threshold level of 10⁻⁴ or 10⁻⁵ or at a rate of between approximately 17% and approximately 64% at a sensitivity threshold level of 10⁻⁶;
(3) wherein said method is effective in obtaining said minimal residual disease (MRD) negative status at a rate of approximately 50% at a sensitivity threshold level of 10⁻⁴ or 10⁻⁵ or at a rate of approximately 39% at a sensitivity threshold level of 10⁻⁶;
(4) wherein said method is effective in obtaining said minimal residual disease (MRD) negative status at a rate of between approximately 66% and approximately 100% in subjects with evaluable samples at a sensitivity threshold level of 10⁻⁵; or
(5) wherein said method is effective in obtaining said minimal residual disease (MRD) negative status at a rate of approximately 100% in subjects with evaluable samples at a sensitivity threshold level of 10⁻⁵;
(6) wherein said method is effective in maintaining a response at a rate of between approximately 5% and approximately 95% at a follow-up time of approximately 6 months, approximately 9 months or approximately 12 months after said administration of said CAR-T cells; or
(7) wherein said method is effective in maintaining a response at a rate of approximately 67% at a follow-up time of approximately 6 months, approximately 9 months or approximately 12 months after said administration of said CAR-T cells;
optionally wherein said method further comprises treating said subject for cytokine release syndrome more than approximately 3 days after said administration of said CAR-T cells, further optionally wherein said method is effective obtaining a rate of recovery from said cytokine release syndrome of between approximately 1% and approximately 100% at a time of approximately 7 days after first observance of said cytokine release syndrome.

59-66. (canceled)

67. The method of claim 2, wherein said method is effective in obtaining at least one response in the subject after said administration of said CAR-T cells, wherein said at least one response comprises, in order from better to worse:
(i) a stringent complete response;
(ii) a complete response;
(iii) a very good partial response;
(iv) a partial response; or
(v) a minimal response;
optionally:
(1) wherein said method is effective in obtaining a first response before a time of between approximately 27 days and approximately 78 days after said administration of said CAR-T cells;
(2) wherein said method is effective in obtaining a first response before a time of between approximately 27 days and approximately 47 days after said administration of said CAR-T cells;
(3) wherein said method is effective in obtaining a first response before approximately 33 days after said administration of said CAR-T cells;
(4) wherein said method is effective in obtaining a first response before approximately 28 days after said administration of said CAR-T cells;
(5) wherein said method is effective in obtaining a best response of any one of minimal response, partial response, very good partial response, complete response or stringent complete response, further optionally wherein said method is effective in obtaining said best response of any one of minimal response, partial response, very good partial response, complete response or stringent complete response at a rate of between approximately 65% and approximately 99%, and further optionally wherein said method is effective in obtaining said best response of any one of minimal response, partial response, very good partial response, complete response or stringent complete response at a rate of approximately 89%;
(6) wherein said method is effective in obtaining a best response of any one of partial response, very good partial response, complete response or stringent complete response, further optionally wherein said method is effective in obtaining said best response of any one of partial response, very good partial response, complete response or stringent complete response at a rate of between approximately 65% and approximately 99%, and further optionally wherein said method is effective in obtaining said best response of any one of partial response, very good partial response, complete response or stringent complete response at a rate of approximately 89%;
(7) wherein said method is effective in obtaining a best response of any one of very good partial response, complete response or stringent complete response, further optionally wherein said method is effective in obtaining said best response of any one of very good partial response, complete response or stringent complete response at a rate of between approximately 41% and approximately 87%, and further optionally wherein said method is effective in obtaining said best response of any one of very good partial response, complete response or stringent complete response at a rate of approximately 67%;
(8) wherein said method is effective in obtaining a best response of complete response or stringent complete response, further optionally wherein said method is effective in obtaining said best response of complete response or stringent complete response at a rate of between and approximately 10% and approximately 54%, and further optionally wherein said method is effective in obtaining said best response of complete response or stringent complete response at a rate of approximately 28%;
(9) wherein said method is effective in obtaining a best response of stringent complete response, further optionally wherein said method is effective in obtaining said best response of stringent complete response at a rate of between approximately 6% and approximately 48%, and further optionally wherein said method is effective in obtaining said best response of stringent complete response at a rate of approximately 22%; or
(10) wherein said method is effective in maintaining a response at a rate of approximately 67% at a follow-up time of approximately 6 months, approximately 9 months or approximately 12 months after said administration of said CAR-T cells;
further optionally:
(A) wherein said method is effective in obtaining said best response before a time of between approximately 27 days and approximately 354 days after said administration of said CAR-T cells;
(B) wherein said method is effective in obtaining said best response before a time of between approximately 27 days and approximately 155 days after said administration of said CAR-T cells;
(C) wherein said method is effective in obtaining said best response before approximately 71 days after said administration of said CAR-T cells;
(D) wherein said method is effective in obtaining said best response before approximately 42 days after said administration of said CAR-T cells;
(E) wherein said method is effective in maintaining a response in the subject at a follow-up time of between the time of said first response and approximately 156 days after said administration of said CAR-T cells; or
(F) wherein said method is effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells, further optionally wherein said method is effective in obtaining either minimal residual disease (MRD) negative complete response or minimal residual disease (MRD) negative stringent complete response at a rate of between approximately 1% and approximately 35% at a follow-up time of approximately 141 days after said administration of said CAR-T cells, and further wherein said method is effective in obtaining either minimal residual disease (MRD) negative complete response or minimal residual disease (MRD) negative stringent complete response at a rate of approximately 11% at a follow-up time of approximately 141 days after said administration of said CAR-T cells.

68-86. (canceled)

87. The method of claim 2, wherein said method is effective in obtaining progression-free survival of the subject, optionally:
(i) wherein said method is effective in obtaining said progression-free survival of the subject at a time between said administration of said CAR-T cells and approximately 182 days after said administration of said CAR-T cells;
(ii) wherein said method is effective in obtaining said progression-free survival at a rate of approximately 100% at a follow-up time of approximately 6 months after said administration of said CAR-T cells;
(iii) wherein said method is effective in obtaining said progression-free survival at a rate of between approximately 5% and approximately 95% at a follow-up time of approximately 9 months or approximately 12 months after said administration of said CAR-T cells; or
(iv) wherein said method is effective in obtaining said progression-free survival at a rate of approximately 67% at a follow-up time of approximately 9 months or approximately 12 months after said administration of said CAR-T cells.

88-103. (canceled)

104. The method of claim 3, wherein:
(i) the subject received prior treatment with at least two, at least four, at least eight or at least twelve prior lines of therapy;
(ii) the subject has relapsed after said at least one prior line of therapy;
(iii) said method is effective in obtaining minimal residual disease (MRD) negative status in said subject assessed in the bone marrow after said administration of said CAR-T cells, optionally:
(1) wherein said minimal residual disease (MRD) negative status is assessed in the bone marrow at a first follow-up time at a first follow-up time of between approximately 56 days and approximately 58 days after said administration of said CAR-T cells, further optionally wherein said method is effective in maintaining said minimal residual disease (MRD) negative status in said subject assessed in the bone marrow at a second follow-up time of between approximately 183 days and approximately 186 days after said administration of said CAR-T cells, further wherein said first follow-up time is earlier than said second follow-up time;
(2) wherein said method is effective in obtaining said minimal residual disease (MRD) negative status at a rate of between approximately 9% and approximately 49% at a sensitivity threshold level of 10⁻⁴, at a rate of between approximately 6% and approximately 44% at a sensitivity threshold level of 10⁻⁵, or at a rate of between approximately 1% and approximately 31% at a sensitivity threshold level of 10⁻⁶;
(3) wherein said method is effective in obtaining said minimal residual disease (MRD) negative status at a rate of approximately 25% at a sensitivity threshold level of 10⁻⁴, at a rate of approximately 20% at a sensitivity threshold level of 10⁻⁵, or at a rate of approximately 10% at a sensitivity threshold level of 10⁻⁶;
(4) wherein said method is effective in obtaining said minimal residual disease (MRD) negative status at a rate of between approximately 22% and approximately 96% in subjects with evaluable samples at a sensitivity threshold level of 10⁻⁵; or
(5) wherein said method is effective in obtaining said minimal residual disease (MRD) negative status at a rate of approximately 67% in subjects with evaluable samples at a sensitivity threshold level of 10⁻⁵;
(iv) wherein said method is effective in obtaining progression-free survival of the subject optionally:
(1) wherein said method is effective in obtaining said progression-free survival of the subject at a time between said administration of said CAR-T cells and approximately 15 days after said administration of said CAR-T cells;
(2) wherein said method is effective in obtaining said progression-free survival of the subject at a time between said administration of said CAR-T cells and approximately 44 days after said administration of said CAR-T cells;
(3) wherein said method is effective in obtaining said progression-free survival of the subject at a time between said administration of said CAR-T cells and approximately 159 days after said administration of said CAR-T cells;
(4) wherein said method is effective in obtaining said progression-free survival at a rate of between approximately 29% and approximately 75% at a follow-up time of approximately 6 months or approximately 9 months after said administration of said CAR-T cells;
(5) wherein said method is effective in obtaining said progression-free survival at a rate of approximately 55% at a follow-up time of approximately 6 months or approximately 9 months after said administration of said CAR-T cells;
(v) wherein said method is effective in obtaining a rate of cytokine release syndrome of between approximately 60% and approximately 99%, optionally wherein said method further comprises treating said subject for cytokine release syndrome more than approximately 3 days after said administration of said CAR-T cells;
(vi) wherein said method is effective in obtaining a rate of immune-effector cell associated neurotoxicity of between approximately 20% and approximately 99%; or
(vii) wherein said at least four medicaments further comprises a proteasomal inhibitor, an immunomodulatory drug and an anti-CD38 antibody.

105-115. (canceled)

116. The method of claim 3, wherein said method is effective in obtaining at least one response in the subject after said administration of said CAR-T cells, wherein said at least one response comprises, in order from better to worse:
(i) a stringent complete response;
(ii) a complete response;
(iii) a very good partial response;
(iv) a partial response; or
(v) a minimal response;
optionally:
(1) wherein said method is effective in obtaining a first response before a time of between approximately 27 days and approximately 153 days after said administration of said CAR-T cells;
(2) wherein said method is effective in obtaining a first response before a time of between approximately 27 days and approximately 88 days after said administration of said CAR-T cells;
(3) wherein said method is effective in obtaining a first response before approximately 43 days after said administration of said CAR-T cells;
(4) wherein said method is effective in obtaining a first response before approximately 28 days after said administration of said CAR-T cells;
(5) wherein said method is effective in obtaining a best response of any one of minimal response, partial response, very good partial response, complete response or stringent complete response, further optionally wherein said method is effective in obtaining said best response of any one of minimal response, partial response, very good partial response, complete response or stringent complete response at a rate of between approximately 23% and approximately 69%, and further optionally wherein said method is effective in obtaining said best response of any one of minimal response, partial response, very good partial response, complete response or stringent complete response at a rate of approximately 45%;
(6) wherein said method is effective in obtaining a best response of any one of partial response, very good partial response, complete response or stringent complete response, further optionally wherein said method is effective in obtaining said best response of any one of partial response, very good partial response, complete response or stringent complete response at a rate of between approximately 19% and approximately 64%, and further optionally wherein said method is effective in obtaining said best response of any one of partial response, very good partial response, complete response or stringent complete response at a rate of approximately 40%;
(7) wherein said method is effective in obtaining a best response of any one of very good partial response, complete response or stringent complete response, further optionally wherein said method is effective in obtaining said best response of any one of very good partial response, complete response or stringent complete response at a rate of between approximately 15% and approximately 59%, and further optionally wherein said method is effective in obtaining said best response of any one of very good partial response, complete response or stringent complete response at a rate of approximately 35%;
(8) wherein said method is effective in obtaining a best response of complete response or stringent complete response, further optionally wherein said method is effective in obtaining said best response of complete response or stringent complete response at a rate of between approximately 3% and approximately 38%;
(9) wherein said method is effective in obtaining a best response of stringent complete response, further optionally wherein said method is effective in obtaining said best response of stringent complete response at a rate of between approximately 1% and approximately 32%;
further optionally:
(A) wherein said method is effective in obtaining said best response before a time of between approximately 27 days and approximately 171 days after said administration of said CAR-T cells;
(B) wherein said method is effective in obtaining said best response before a time of between approximately 27 days and approximately 133 days after said administration of said CAR-T cells;
(C) wherein said method is effective in obtaining said best response before approximately 78 days after said administration of said CAR-T cells;
(D) wherein said method is effective in obtaining said best response before approximately 56 days after said administration of said CAR-T cells;
(E) wherein said method is effective in maintaining a response in the subject at a follow-up time of between the time of said first response and approximately 132 days after said administration of said CAR-T cells, further optionally wherein said first response was obtained between the time of said administration of said CAR-T cells and approximately 131 days after said administration of said CAR-T cells;
(F) wherein said method is effective in maintaining a response at a rate of between approximately 20% and approximately 96% at a follow-up time of approximately 6 months after said administration of said CAR-T cells; or
(G) wherein said method is effective in maintaining a response at a rate of approximately 80% at a follow-up time of approximately 6 months after said administration of said CAR-T cells.

117-150. (canceled)

151. The method of claim 1, wherein:
(i) the multiple myeloma is refractory to at least two, at least three, at least four, at least five medicaments;
(ii) the subject has bone marrow plasma cells of between approximately 10% and approximately 30% before said administration of said CAR-T cells;
(iii) wherein the dose comprises 1.0 × 10⁵ to 5.0 × 10⁶ of said CAR-T cells per kilogram of the mass of the subject;
(iv) the dose comprises 5.0 × 10⁵ to 1.0 × 10⁶ of said CAR-T cells per kilogram of the mass of the subject;
(v) the dose comprises approximately 0.75 × 10⁶ of said CAR-T cells per kilogram of the mass of the subject;
(vi) the dose comprises less than 1.0 × 10⁸ of said CAR-T cells per subject;
(vii) said administration of said CAR-T cells is via a single intravenous infusion, optionally:
(1) wherein said single intravenous infusion is administered using a single bag of said CAR-T cells, further optionally wherein said administration of said single bag of said CAR-T cells is completed between the time at which said single bag of CAR-T cells is thawed and three hours after said single bag of CAR-T cells is thawed; or
(2) wherein said single intravenous administration is administered using two bags of said CAR-T cells, further optionally wherein said administration of each of said two bags of said CAR-T cells is completed between the time at which a first bag of said two bags of CAR-T cells is thawed and three hours after said first bag of CAR-T cells is thawed;
(viii) a lymphodepleting regimen precedes said administration of CAR-T cells by approximately 5 days to approximately 7 days, further optionally wherein said lymphodepleting regimen is administered intravenously, further optionally wherein said lymphodepleting regimen comprises:
(a) administration of cyclophosphamide; or
(b) administration of fludarabine;
further optionally wherein said cyclophosphamide is administered intravenously at 300 mg/m2, further optionally wherein said fludarabine is administered intravenously at 30 mg/m2, further optionally wherein the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject, further optionally wherein the subject had an increase in tumor burden despite said bridging therapy, and further optionally wherein the subject had an increase in tumor burden of approximately 25% or greater despite said bridging therapy;
(ix) a lymphodepleting regimen comprising cyclophosphamide administered intravenously at 300 mg/m² and fludarabine administered intravenously at 30 mg/m2 precedes said administration of CAR-T cells by approximately 5 days to approximately 7 days;
(x) the subject is treated with pre-administration medication comprising an antipyretic and an antihistamine up to approximately 1 hour before said administration of said CAR-T cells, optionally:
(1) wherein said antipyretic comprises either paracetamol or acetaminophen;
(2) wherein said antipyretic is administered to the subject either orally or intravenously;
(3) wherein said antipyretic is administered to the subject at a dosage of between 650 mg and 1000 mg;
(4) wherein said antihistamine comprises diphenhydramine;
(5) wherein said antihistamine is administered to the subject either orally or intravenously;
(6) wherein said antihistamine is administered at a dosage of between 25 mg and 50 mg, or its equivalent;
(7) wherein said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent;
(xi) the composition comprising CAR-T cells administered to the subject further comprises an excipient selected from dimethylsulfoxide or dextran-40;
(xii) the subject has had no prior exposure to a BCMA-targeting medicament; or
(xiii) the multiple myeloma is progressive.

152-188. (canceled)

189. The method of claim 1, wherein:
(i) the first BCMA binding moiety and/or the second BCMA binding moiety is an anti-BCMA VHH, optionally wherein the first BCMA binding moiety is a first anti-BCMA VHH and the second BCMA binding moiety is a second anti-BCMA VHH;
(ii) the first BCMA binding moiety comprises the amino acid sequence of SEQ ID NO: 2;
(iii) the first BCMA binding moiety comprises a polypeptide encoded by the nucleic acid sequence of SEQ ID NO: 10;
(iv) the second BCMA binding moiety comprises the amino acid sequence of SEQ ID NO: 4;
(v) the second BCMA binding moiety comprises a polypeptide encoded by the nucleic acid sequence of SEQ ID NO: 12;
(vi) the first BCMA binding moiety and the second BCMA binding moiety are connected to each other via a peptide linker, optionally wherein the peptide linker comprises the amino acid sequence of SEQ ID NO: 3, and optionally wherein the peptide linker comprises a polypeptide encoded by the nucleic acid sequence of SEQ ID NO: 11;
(vii) the CAR polypeptide further comprises a signal peptide located at the N-terminus of the polypeptide, optionally wherein the signal peptide is derived from CD8-alpha, optionally wherein the signal peptide comprises the amino acid sequence of SEQ ID NO: 1, and optionally wherein the signal peptide comprises a polypeptide encoded by the nucleic acid sequence of SEQ ID NO: 9;
(viii) the transmembrane domain comprises the amino acid sequence of SEQ ID NO: 6;
(ix) the transmembrane domain comprises a polypeptide encoded by the nucleic acid sequence of SEQ ID NO: 14;
(x) the intracellular signaling domain comprises a primary intracellular signaling domain of an immune effector cell;
(xi) the intracellular signaling domain is derived from CD3ζ;
(xii) the intracellular signaling domain comprises at least one co-stimulatory signaling domains;
(xiii) the intracellular signaling domain comprises an amino acid sequence of SEQ ID NO: 8;
(xiv) the intracellular signaling domain comprises a polypeptide encoded by the nucleic acid sequence of SEQ ID NO: 16;
(xv) the intracellular signaling domain comprises an amino acid sequence of SEQ ID NO: 7;
(xvi) the intracellular signaling domain comprises a polypeptide encoded by the nucleic acid sequence of SEQ ID NO: 15;
(xvii) the CAR polypeptide further comprises a hinge domain located between the C-terminus of the extracellular antigen binding domain and the N-terminus of the transmembrane domain, optionally wherein the hinge domain comprises the amino acid sequence of SEQ ID NO: 5, and optionally wherein the hinge domain comprises a polypeptide encoded by the nucleic acid sequence of SEQ ID NO: 13;
(xviii) the CAR comprises the amino acid sequence of SEQ ID NO: 17;
(xix) the T cells are autologous T cells; or
(xx) the T cells are allogeneic T cells.

190-216. (canceled)

217. The method of claim 1, wherein the subject is human.

218-219. (canceled)

220. The method of claim 2, wherein:
(i) the multiple myeloma is refractory to at least two, at least three, at least four, at least five medicaments;
(ii) the subject has bone marrow plasma cells of between approximately 10% and approximately 30% before said administration of said CAR-T cells;
(iii) wherein the dose comprises 1.0 × 10⁵ to 5.0 × 10⁶ of said CAR-T cells per kilogram of the mass of the subject;
(iv) the dose comprises 5.0 × 10⁵ to 1.0 × 10⁶ of said CAR-T cells per kilogram of the mass of the subject;
(v) the dose comprises approximately 0.75 × 10⁶ of said CAR-T cells per kilogram of the mass of the subject;
(vi) the dose comprises less than 1.0 × 10⁸ of said CAR-T cells per subject;
(vii) said administration of said CAR-T cells is via a single intravenous infusion, optionally:
(1) wherein said single intravenous infusion is administered using a single bag of said CAR-T cells, further optionally wherein said administration of said single bag of said CAR-T cells is completed between the time at which said single bag of CAR-T cells is thawed and three hours after said single bag of CAR-T cells is thawed; or
(2) wherein said single intravenous administration is administered using two bags of said CAR-T cells, further optionally wherein said administration of each of said two bags of said CAR-T cells is completed between the time at which a first bag of said two bags of CAR-T cells is thawed and three hours after said first bag of CAR-T cells is thawed;
(viii) a lymphodepleting regimen precedes said administration of CAR-T cells by approximately 5 days to approximately 7 days, further optionally wherein said lymphodepleting regimen is administered intravenously, further optionally wherein said lymphodepleting regimen comprises:
(a) administration of cyclophosphamide; or
(b) administration of fludarabine;
further optionally wherein said cyclophosphamide is administered intravenously at 300 mg/m2, further optionally wherein said fludarabine is administered intravenously at 30 mg/m2, further optionally wherein the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject, further optionally wherein the subject had an increase in tumor burden despite said bridging therapy, and further optionally wherein the subject had an increase in tumor burden of approximately 25% or greater despite said bridging therapy;
(ix) a lymphodepleting regimen comprising cyclophosphamide administered intravenously at 300 mg/m² and fludarabine administered intravenously at 30 mg/m² precedes said administration of CAR-T cells by approximately 5 days to approximately 7 days;
(x) the subject is treated with pre-administration medication comprising an antipyretic and an antihistamine up to approximately 1 hour before said administration of said CAR-T cells, optionally:
(1) wherein said antipyretic comprises either paracetamol or acetaminophen;
(2) wherein said antipyretic is administered to the subject either orally or intravenously;
(3) wherein said antipyretic is administered to the subject at a dosage of between 650 mg and 1000 mg;
(4) wherein said antihistamine comprises diphenhydramine;
(5) wherein said antihistamine is administered to the subject either orally or intravenously;
(6) wherein said antihistamine is administered at a dosage of between 25 mg and 50 mg, or its equivalent;
(7) wherein said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent;
(xi) the composition comprising CAR-T cells administered to the subject further comprises an excipient selected from dimethylsulfoxide or dextran-40;
(xii) the subject has had no prior exposure to a BCMA-targeting medicament; or
(xiii) the multiple myeloma is progressive.

221. The method of claim 2, wherein:
(i) the first BCMA binding moiety and/or the second BCMA binding moiety is an anti-BCMA VHH, optionally wherein the first BCMA binding moiety is a first anti-BCMA VHH and the second BCMA binding moiety is a second anti-BCMA VHH;
(ii) the first BCMA binding moiety comprises the amino acid sequence of SEQ ID NO: 2;
(iii) the first BCMA binding moiety comprises a polypeptide encoded by the nucleic acid sequence of SEQ ID NO: 10;
(iv) the second BCMA binding moiety comprises the amino acid sequence of SEQ ID NO: 4;
(v) the second BCMA binding moiety comprises a polypeptide encoded by the nucleic acid sequence of SEQ ID NO: 12;
(vi) the first BCMA binding moiety and the second BCMA binding moiety are connected to each other via a peptide linker, optionally wherein the peptide linker comprises the amino acid sequence of SEQ ID NO: 3, and optionally wherein the peptide linker comprises a polypeptide encoded by the nucleic acid sequence of SEQ ID NO: 11;
(vii) the CAR polypeptide further comprises a signal peptide located at the N-terminus of the polypeptide, optionally wherein the signal peptide is derived from CD8-alpha, optionally wherein the signal peptide comprises the amino acid sequence of SEQ ID NO: 1, and optionally wherein the signal peptide comprises a polypeptide encoded by the nucleic acid sequence of SEQ ID NO: 9;
(viii) the transmembrane domain comprises the amino acid sequence of SEQ ID NO: 6;
(ix) the transmembrane domain comprises a polypeptide encoded by the nucleic acid sequence of SEQ ID NO: 14;
(x) the intracellular signaling domain comprises a primary intracellular signaling domain of an immune effector cell;
(xi) the intracellular signaling domain is derived from CD3ζ;
(xii) the intracellular signaling domain comprises at least one co-stimulatory signaling domains;
(xiii) the intracellular signaling domain comprises an amino acid sequence of SEQ ID NO: 8;
(xiv) the intracellular signaling domain comprises a polypeptide encoded by the nucleic acid sequence of SEQ ID NO: 16;
(xv) the intracellular signaling domain comprises an amino acid sequence of SEQ ID NO: 7;
(xvi) the intracellular signaling domain comprises a polypeptide encoded by the nucleic acid sequence of SEQ ID NO: 15;
(xvii) the CAR polypeptide further comprises a hinge domain located between the C-terminus of the extracellular antigen binding domain and the N-terminus of the transmembrane domain, optionally wherein the hinge domain comprises the amino acid sequence of SEQ ID NO: 5, and optionally wherein the hinge domain comprises a polypeptide encoded by the nucleic acid sequence of SEQ ID NO: 13;
(xviii) the CAR comprises the amino acid sequence of SEQ ID NO: 17;
(xix) the T cells are autologous T cells; or
(xx) the T cells are allogeneic T cells.

222. The method of claim 2, wherein the subject is human.

223. The method of claim 3, wherein:
(i) the multiple myeloma is refractory to at least two, at least three, at least four, at least five medicaments;
(ii) the subject has bone marrow plasma cells of between approximately 10% and approximately 30% before said administration of said CAR-T cells;
(iii) wherein the dose comprises 1.0 × 10⁵ to 5.0 × 10⁶ of said CAR-T cells per kilogram of the mass of the subject;
(iv) the dose comprises 5.0 × 10⁵ to 1.0 × 10⁶ of said CAR-T cells per kilogram of the mass of the subject;
(v) the dose comprises approximately 0.75 × 10⁶ of said CAR-T cells per kilogram of the mass of the subject;
(vi) the dose comprises less than 1.0 × 10⁸ of said CAR-T cells per subject;
(vii) said administration of said CAR-T cells is via a single intravenous infusion, optionally:
(1) wherein said single intravenous infusion is administered using a single bag of said CAR-T cells, further optionally wherein said administration of said single bag of said CAR-T cells is completed between the time at which said single bag of CAR-T cells is thawed and three hours after said single bag of CAR-T cells is thawed; or
(2) wherein said single intravenous administration is administered using two bags of said CAR-T cells, further optionally wherein said administration of each of said two bags of said CAR-T cells is completed between the time at which a first bag of said two bags of CAR-T cells is thawed and three hours after said first bag of CAR-T cells is thawed;
(viii) a lymphodepleting regimen precedes said administration of CAR-T cells by approximately 5 days to approximately 7 days, further optionally wherein said lymphodepleting regimen is administered intravenously, further optionally wherein said lymphodepleting regimen comprises:
(a) administration of cyclophosphamide; or
(b) administration of fludarabine;
further optionally wherein said cyclophosphamide is administered intravenously at 300 mg/m2, further optionally wherein said fludarabine is administered intravenously at 30 mg/m2, further optionally wherein the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject, further optionally wherein the subject had an increase in tumor burden despite said bridging therapy, and further optionally wherein the subject had an increase in tumor burden of approximately 25% or greater despite said bridging therapy;
(ix) a lymphodepleting regimen comprising cyclophosphamide administered intravenously at 300 mg/m² and fludarabine administered intravenously at 30 mg/m² precedes said administration of CAR-T cells by approximately 5 days to approximately 7 days;
(x) the subject is treated with pre-administration medication comprising an antipyretic and an antihistamine up to approximately 1 hour before said administration of said CAR-T cells, optionally:
(1) wherein said antipyretic comprises either paracetamol or acetaminophen;
(2) wherein said antipyretic is administered to the subject either orally or intravenously;
(3) wherein said antipyretic is administered to the subject at a dosage of between 650 mg and 1000 mg;
(4) wherein said antihistamine comprises diphenhydramine;
(5) wherein said antihistamine is administered to the subject either orally or intravenously;
(6) wherein said antihistamine is administered at a dosage of between 25 mg and 50 mg, or its equivalent;
(7) wherein said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent;
(xi) the composition comprising CAR-T cells administered to the subject further comprises an excipient selected from dimethylsulfoxide or dextran-40;
(xii) the subject has had no prior exposure to a BCMA-targeting medicament; or
(xiii) the multiple myeloma is progressive.

224. The method of claim 3, wherein:
(i) the first BCMA binding moiety and/or the second BCMA binding moiety is an anti-BCMA VHH, optionally wherein the first BCMA binding moiety is a first anti-BCMA VHH and the second BCMA binding moiety is a second anti-BCMA VHH;
(ii) the first BCMA binding moiety comprises the amino acid sequence of SEQ ID NO: 2;
(iii) the first BCMA binding moiety comprises a polypeptide encoded by the nucleic acid sequence of SEQ ID NO: 10;
(iv) the second BCMA binding moiety comprises the amino acid sequence of SEQ ID NO: 4;
(v) the second BCMA binding moiety comprises a polypeptide encoded by the nucleic acid sequence of SEQ ID NO: 12;
(vi) the first BCMA binding moiety and the second BCMA binding moiety are connected to each other via a peptide linker, optionally wherein the peptide linker comprises the amino acid sequence of SEQ ID NO: 3, and optionally wherein the peptide linker comprises a polypeptide encoded by the nucleic acid sequence of SEQ ID NO: 11;
(vii) the CAR polypeptide further comprises a signal peptide located at the N-terminus of the polypeptide, optionally wherein the signal peptide is derived from CD8-alpha, optionally wherein the signal peptide comprises the amino acid sequence of SEQ ID NO: 1, and optionally wherein the signal peptide comprises a polypeptide encoded by the nucleic acid sequence of SEQ ID NO: 9;
(viii) the transmembrane domain comprises the amino acid sequence of SEQ ID NO: 6;
(ix) the transmembrane domain comprises a polypeptide encoded by the nucleic acid sequence of SEQ ID NO: 14;
(x) the intracellular signaling domain comprises a primary intracellular signaling domain of an immune effector cell;
(xi) the intracellular signaling domain is derived from CD3ζ;
(xii) the intracellular signaling domain comprises at least one co-stimulatory signaling domains;
(xiii) the intracellular signaling domain comprises an amino acid sequence of SEQ ID NO: 8;
(xiv) the intracellular signaling domain comprises a polypeptide encoded by the nucleic acid sequence of SEQ ID NO: 16;
(xv) the intracellular signaling domain comprises an amino acid sequence of SEQ ID NO: 7;
(xvi) the intracellular signaling domain comprises a polypeptide encoded by the nucleic acid sequence of SEQ ID NO: 15;
(xvii) the CAR polypeptide further comprises a hinge domain located between the C-terminus of the extracellular antigen binding domain and the N-terminus of the transmembrane domain, optionally wherein the hinge domain comprises the amino acid sequence of SEQ ID NO: 5, and optionally wherein the hinge domain comprises a polypeptide encoded by the nucleic acid sequence of SEQ ID NO: 13;
(xviii) the CAR comprises the amino acid sequence of SEQ ID NO: 17;
(xix) the T cells are autologous T cells; or
(xx) the T cells are allogeneic T cells.

225. The method of claim 3, wherein the subject is human.
